(19) United States
(12) Patent Application Publication
(10) Pub. No.: US 2007/0275871 A1
(43) Pub. Date: Nov. 29, 2007

(54) Epo Mimetic Peptides and Fusion Proteins

(75) Inventors: Homayoun Sadeghi, King of Prussia, PA (US); Andrew J. Turner, King of Prussia, PA (US)

Correspondence Address: Pfizer Inc.;Patent Department, MS 8260-1611, Eastern Point Road, Groton, CT, 06340, US

(73) Assignee: BIOREXIS TECHNOLOGY, INC., Wilmington, DE (US)

(21) Appl. No.: 10/569,276

(22) PCT Filed: Aug. 30, 2004
(86) PCT No.: PCT/US04/27949

§ 371 Date: Aug. 3, 2007

Related U.S. Application Data
(60) Provisional application No. 60/551,552, filed on Mar. 10, 2004.

(30) Foreign Application Priority Data
Aug. 28, 2003 (US) PCT/US03/26818

(51) Int. Cl. A61K 38/17; A01K 67/033; C07H 19/073; C07K 14/505

(52) U.S. Cl. 514/2; 435/255.1; 435/320.1; 435/325; 514/8; 530/350; 530/380; 530/397; 536/23.4; 800/4; 800/8

(57) ABSTRACT

EPM peptides, including EPM peptide-fusion proteins with increased serum half-life or serum stability are disclosed. Compositions comprising the EPM peptides or fusion proteins and methods of treating or preventing disorders by administering a therapeutically or prophylactically effective amount of an EPM peptide or fusion protein to a patient in need thereof are also disclosed.

### RELATED APPLICATIONS

This application is a continuation-in-part of PCT International Application No. PCT/US03/26818, filed Aug. 28, 2003, and claims the benefit of U.S. Provisional Application No. 60/551,552, filed Mar. 10, 2004, both of which are incorporated herein by reference in their entirety.

### FIELD OF THE INVENTION

The invention relates to biologically active peptides, their preparation, pharmaceutical compositions comprising them and methods of use thereof. In particular the invention relates to EPO peptide mimetic (“EPM”) peptides and modified EPM peptides fused to or inserted in a second peptide or protein to generate fusion proteins of the invention. The invention also relates to compositions comprising the EPM peptides or fusion proteins and methods of treating or preventing disorders by administering a therapeutically or prophylactically effective amount of an EPM peptide or fusion protein to a patient in need thereof.

### BACKGROUND OF THE INVENTION

Therapeutic Proteins and Peptides

Therapeutic proteins or peptides in their native state or when recombinantly produced are typically labile molecules exhibiting short periods of serum stability or short in vivo circulatory half-lives. In addition, these molecules are often extremely labile when formulated, particularly when formulated in aqueous solutions for diagnostic and therapeutic purposes.

Few practical solutions exist to extend or promote the stability in vivo or in vitro of proteinaceous therapeutic molecules. Polyethylene glycol (PEG) is a substance that can be attached to a protein, resulting in longer-acting, sustained activity of the protein. If the activity of a protein is prolonged by the attachment to PEG, the frequency that the protein needs to be administered may be decreased. PEG attachment, however, often decreases or destroys the protein's therapeutic activity. While in some instances PEG attachment can reduce immunogenicity of the protein, in other instances it may increase immunogenicity.

Therapeutic proteins or peptides have also been stabilized by fusion to a protein capable of extending the in vivo circulatory half-life of the therapeutic protein. For instance, therapeutic proteins fused to albumin or to antibody fragments may exhibit extended in vivo circulatory half-life when compared to the therapeutic protein in the unfused state. See U.S. Pat. Nos. 5,876,969 and 5,766,883.

Erythropoietin Mimetic Peptide (EMP)

Erythropoietin (EPO) is a glycoprotein that is synthesized in the kidneys of mammals for stimulating mitotic cell division and differentiation of erythrocyte precursor cells. Accordingly, EPO acts to stimulate and regulate the production of erythrocytes. Because of its role in red blood cell formation, EPO is useful in both the diagnosis and the treatment of blood disorders characterized by low or defective red blood cell production.

Studies have shown the efficacy of EPO therapy in a variety of disease states, disorders, and states of hematologic irregularity, for example, beta-thalassemia (Vedovato et al. (1984) Acta. Haematol. 71:211-213); cystic fibrosis (Vichinsky et al. (1984) J. Pediatric 105:15-21); pregnancy and menstrual disorders (Cotes et al. (1983) Brit. J. Ostet. Gyneacol. 90:304-311); early anemia of prematurity (Haga et al. (1983) Acta Pediatr. Scand. 72:827-831); spinal cord injury (Claus-Walker et al. (1984) Arch. Phys. Med. Rehabil. 65:370-374); space flight (Dunn et al. (1984) Eur. J. Appl. Physiol. 52:178-182); acute blood loss (Miller et al. (1982) Brit. J. Haematol. 52:545-590); aging (Udupa et al. (1984) J. Lab. Clin. Med. 103:574-588); various neoplastic disease states accompanied by abnormal erythropoiesis (Dainiak et al. (1983) Cancer 5:1101-1106); and renal insufficiency (Eschbach et al. (1987) N. Eng. J. Med. 316:73-78). During the last fifteen years, EPO has been used for the treatment of the anemia of renal failure, anemia of chronic disease associated with rheumatoid arthritis, inflammatory bowel disease, AIDS, and cancer, as well as for the treatment of anemia in hematopoietic malignancies, post-bone marrow transplantation, and autologous blood donation.

The activity of EPO is mediated by its receptor. The EPO-receptor (EPO-R) belongs to the class of growth-factor-type receptors which are activated by a ligand-induced protein dimerization. Other hormones and cytokines such as human growth hormone (hGH), granulocyte colony stimulating factor (G-CSF), epidermal growth factor (EGF) and insulin can cross-link two receptors resulting in juxtaposition of two cytoplasmic tails. Many of these dimerization-activated receptors have protein kinase domains within the cytoplasmic tails that phosphorylate the neighboring tail upon dimerization. While some cytoplasmic tails lack intrinsic kinase activity, these function by association with protein kinases. The EPO receptor is of the latter type. In each case, phosphorylation results in the activation of a signaling pathway.

There has been an increasing interest in molecular mimicry with EPO potency. For example, dimerization of the erythropoietin receptor (EPOR) in the presence of either natural EPO or synthetic EPO mimetic peptides (EMPs) is the extracellular event that leads to activation of the receptor and downstream signal transduction events. In general, there is an interest in obtaining mimetics with equivalent potency to EPO.

Wrighton et al (1996, Science, 273:458-463) employed phage display where random peptides are exposed on coat proteins of filamentous phage. A library of random peptide-phage was allowed to bind to and subsequently eluted from the extracellular domain of EPO receptor in the screening system. They used a weak-binding system to first fish out EPO domain-weak-binding (Kd 10 mM) CRIGPITWVC (SEQ ID NO: 14) as the consensus sequence. Consequently, a 20-amino acid peptide, EMP-1, (GGTYSCHFGPLTWVCKPQGG, SEQ ID NO: 4) with an affinity (Kd) of 200 nM, compared to 200 pM for EPO was isolated, the sequence of which does not actually exist in the native EPO. The crystal structure at 2.8 Å resolution of a complex of this mimetic agonist peptide with the extracellular domain of EPO receptor revealed that a peptide dimer induces an almost perfect twofold dimerization of the receptor (Livnah et al., 1996 Science, 273 (274): 464-471). This 20-amino acid peptide has a β-sheet structure and is stabilized by the C—C disulfide bond.

The biological activity of EMP-1 indicates that EMP-1 can act as an EPO mimetic. For example, EMP-1 competes with EPO in receptor binding assays to cause cellular proliferation of cell lines engineered to be responsive to EPO (Wrighton et al., 1996, Science, 273:458-463). Both EPO and EMP-1 induce a similar cascade of phosphorylation events and cell cycle progression in EPO responsive cells (Wrighton et al., 1996, Science, 273:458-463). Further, EMP-1 demonstrates significant erythropoietic effects in mice as monitored by two different in vivo assays of nascent red blood cell production (Wrighton et al., 1996, Science, 273:458-463).

Johnson et al. (1998, Biochemistry, 37:3699-3710) identified the minimal peptide that retained activity in the assays for EPO mimetic action. Using N- and C-terminal deletions, they found that the minimal active peptide is EMP-20 having the sequence, YSCHFGPLTWVCK, namely amino acids 4 through 16 of EMP-1 (SEQ ID NO: 4). They also found Tyr4 and Trp13 of EMP-1 to be critical for mimetic action. The two cysteine residues at positions 3 and 12 are also essential for peptide activity as they are responsible for the C—C disulfide bond that stabilizes the 3-dimensional structure of the peptide.

### SUMMARY OF THE INVENTION

The invention encompasses modified erythropoietin (“EPO”) peptide mimetic (“EPM”) peptides, which comprise a mutation or variation in the EMP-1 peptide's amino acid sequence.

Another embodiment of the invention encompasses a fusion protein comprising one or more EPM peptides fused to a second peptide or protein, wherein the EPM peptide exhibits increased serum stability or in vivo circulatory half-life compared to EMP-1.

Another embodiment of the invention encompasses pharmaceutical formulations, compositions, and dosage forms comprising an EPM peptide or a fusion protein comprising an EPM peptide.

Another embodiment of the invention encompasses methods of treating or preventing a disorder comprising administering to a patient in need of such treatment or prevention an EPM peptide or a fusion protein comprising an EPM peptide.

Another embodiment of the invention encompasses methods of extending the serum stability, in vivo circulatory half-life, and bioavailability of an EPM peptide.

### BRIEF DESCRIPTION OF THE DRAWINGS

FIG. 1 shows an alignment of the N and C Domains of Human (Hu) transferrin (Tf) (SEQ ID NO: 3) with similarities and identities highlighted.

FIGS. 2A-2B show an alignment of transferrin sequences from different species. Light shading: Similarity; Dark shading: Identity (SEQ ID NOS: 15-21).

FIG. 3 shows the location of a number of Tf surface exposed insertion sites for therapeutic proteins, polypeptides or peptides.

FIG. 4 shows pREX0052.

FIG. 5 shows pREX0387.

FIG. 6 shows pREX0155.

FIG. 7 shows pREX0341.

FIG. 8 shows pREX0607.

FIG. 9 shows pREX0242.

FIG. 10 shows pREX0317.

FIG. 11 shows pREX0413.

FIG. 12 shows pREX0318.

### DETAILED DESCRIPTION

Definitions

As used herein, an “amino acid corresponding to” or an “equivalent amino acid” in a transferrin sequence is identified by alignment to maximize the identity or similarity between a first transferrin sequence and at least a second transferrin sequence. The number used to identify an equivalent amino acid in a second transferrin sequence is based on the number used to identify the corresponding amino acid in the first transferrin sequence. In certain cases, these phrases may be used to describe the amino acid residues in human transferrin compared to certain residues in rabbit serum transferrin.

As used herein, the term “biological activity” refers to a function or set of activities performed by a therapeutic molecule, protein or peptide in a biological context (i.e., in an organism or an in vitro facsimile thereof). Biological activities may include but are not limited to the functions of the therapeutic molecule portion of the claimed fusion proteins, such as, but not limited to, the induction of extracellular matrix secretion from responsive cell lines, the induction of hormone secretion, the induction of chemotaxis, the induction of mitogenesis, the induction of differentiation, or the inhibition of cell division of responsive cells. A fusion protein or peptide of the invention is considered to be biologically active if it exhibits one or more biological activities of EMP-1 or EPO.

As used herein, “binders” are agents used to impart cohesive qualities to the powdered material. Binders, or “granulators” as they are sometimes known, impart a cohesiveness to the tablet formulation, which ensures the tablet remains intact after compression, as well as improving the free-flowing qualities by the formulation of granules of desired hardness and size. Materials commonly used as binders include starch; gelatin; sugars, such as sucrose, glucose, dextrose, molasses, and lactose; natural and synthetic gums, such as acacia, sodium alginate, extract of Irish moss, panwar gum, ghatti gum, mucilage of isapol husks, carboxymethylcellulose, methylcellulose, polyvinylpyrrolidone, Veegum, microcrystalline cellulose, microcrystalline dextrose, amylose, and larch arabogalactan, and the like.

As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxy-methyl, pivaloyloxymethyl, and pivaloyloxyethyl esters), lactonyl esters (such as phthalidyl and thiophthalidyl esters), lower alkoxyacyloxyalkyl esters (such as methoxycarbonyloxy-methyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, a amino acid amides, alkoxyacyl amides, and alkylaminoalkyl-carbonyl amides. Examples of biohydrolyzable carbamates include, but are not limited to, lower alkylamines, substituted ethylenediamines, aminoacids, hydroxyalkylamines, heterocyclic and heteroaromatic amines, and polyether amines.

As used herein, the term “carrier” refers to a diluent, adjuvant, excipient, or vehicle with which a composition is administered. Such pharmaceutical carriers can be sterile liquids, such as water and oils, including those of petroleum, animal, vegetable or synthetic origin, such as peanut oil, soybean oil, mineral oil, sesame oil and the like.

As used herein, “coloring agents” are agents that give tablets a more pleasing appearance, and in addition help the manufacturer to control the product during its preparation and help the user to identify the product. Any of the approved certified water-soluble FD&C dyes, mixtures thereof, or their corresponding lakes may be used to color tablets. A color lake is the combination by adsorption of a water-soluble dye to a hydrous oxide of a heavy metal, resulting in an insoluble form of the dye.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, Van der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

As used herein and unless otherwise indicated, the term “compositions of the invention” refers to an EPM peptide or fusion protein of the invention or pharmaceutically acceptable salts, solvates, hydrates, clathrates, polymorphs and prodrugs thereof and a pharmaceutically acceptable vehicle.

As used herein the term “conservative amino acid substitution” refers to a substitution in which an amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

As used herein, “diluents” are inert substances added to increase the bulk of the formulation to make the tablet a practical size for compression. Commonly used diluents include calcium phosphate, calcium sulfate, lactose, kaolin, mannitol, sodium chloride, dry starch, powdered sugar, silica, and the like.

As used herein, “disintegrators” or “disintegrants” are substances that facilitate the breakup or disintegration of tablets after administration. Materials serving as disintegrants have been chemically classified as starches, clays, celluloses, algins, or gums. Other disintegrators include Veegum HV, methylcellulose, agar, bentonite, cellulose and wood products, natural sponge, cation-exchange resins, alginic acid, guar gum, citrus pulp, cross-linked polyvinylpyrrolidone, carboxymethylcellulose, and the like.

As used herein the phrase “disorders and disease states of hematological irregularity” refers to any disorder that deals with diseases of the blood and blood-forming organs. Examples of disorders and disease states of hematological irregularity include, but are not limited to, anemia, beta-thalassemia, cystic fibrosis, pregnancy and menstrual disorders, early anemia of prematurity, spinal cord injury, acute blood loss, aging, neoplastic disease states associated with abnormal erythropoiesis, and renal insufficiency.

The term “dispersibility” or “dispersible” means a dry powder having a moisture content of less than about 10% by weight (% w) water, usually below about 5% w and preferably less than about 3% w; a particle size of about 1.0-5.0 μm mass median diameter (MMD), usually 1.0-4.0 μm MMD, and preferably 1.0-3.0 μm MMD; a delivered dose of about >30%, usually >40%, preferably >50%, and most preferred >60%; and an aerosol particle size distribution of 1.0-5.0 μm mass median aerodynamic diameter (MMAD), usually 1.5-4.5 μm MMAD, and preferably 1.5-4.0 μm MMAD.

The term “dry” means that the composition has a moisture content such that the particles are readily dispersible in an inhalation device to form an aerosol. This moisture content is generally below about 10% by weight (% w) water, usually below about 5% w and preferably less than about 3% w.

As used herein, “effective amount” means an amount of a drug or pharmacologically active agent that is sufficient to provide the desired local or systemic effect and performance at a reasonable benefit/risk ratio attending any medical treatment.

As used herein, “EMP-1 activity” refers to the ability of a EPM peptide or fusion protein of the invention to mimic the activity of the protein hormone, EPO. EMP-1 activity further refers to the affinity of an EPM peptide or fusion protein of the invention for the erythropoietin receptor (EPOR) and correspondingly elevated potency in cell-based assays. It further includes activation of an EPO receptor, for example, induced by binding of an EPM peptide or fusion protein ligand to a specific ligand-binding domain on the receptor. EMP-1 activity further includes, but is not limited to, interaction of an EPM peptide or fusion protein of the invention directly with the receptor for erythropoietin on red blood cell precursors, which can stimulate red cell formation with similar potency to erythropoietin.

As used herein, “flavoring agents” vary considerably in their chemical structure, ranging from simple esters, alcohols, and aldehydes to carbohydrates and complex volatile oils. Synthetic flavors of almost any desired type are now available.

As used herein, the terms “fragment of a Tf protein” or “Tf protein,” or “portion of a Tf protein” refer to an amino acid sequence comprising at least about 5%, 10%, 20%, 30%, 40%, 50%, 60%, 70%, 80%, 90%, 95%, 96%, 97%, 98%, 99% or 100% of a naturally occurring Tf protein or mutant thereof.

The invention also provides modified EPM fusion or chimeric proteins. As used herein, a modified EPM “fusion protein” or “chimeric protein” comprises an EPM peptide operatively linked to a second peptide or protein.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters. In addition, the terms “gene” and “recombinant gene” also refer to nucleic acid molecules comprising an open reading frame encoding an EMP-1 protein, preferably a mammalian EMP-1 protein.

As used herein, a “heterologous polynucleotide” or a “heterologous nucleic acid” or a “heterologous gene” or a “heterologous sequence” or an “exogenous DNA segment” refers to a polynucleotide, nucleic acid or DNA segment that originates from a source foreign to the particular host cell, or, if from the same source, is modified from its original form. A heterologous gene in a host cell includes a gene that is endogenous to the particular host cell, but has been modified. Thus, the terms refer to a DNA segment which is foreign or heterologous to the cell, or homologous to the cell but in a position within the host cell nucleic acid in which the element is not ordinarily found. As an example, a signal sequence native to a yeast cell but attached to a human Tf sequence is heterologous.

As used herein, an “isolated” nucleic acid sequence refers to a nucleic acid sequence which is essentially free of other nucleic acid sequences, e.g., at least about 20% pure, preferably at least about 40% pure, more preferably about 60% pure, even more preferably about 80% pure, most preferably about 90% pure, and even most preferably about 95% pure, as determined by agarose gel electrophoresis. For example, an isolated nucleic acid sequence can be obtained by standard cloning procedures used in genetic engineering to relocate the nucleic acid sequence from its natural location to a different site where it will be reproduced. The cloning procedures may involve excision and isolation of a desired nucleic acid fragment comprising the nucleic acid sequence encoding the polypeptide, insertion of the fragment into a vector molecule, and incorporation of the recombinant vector into a host cell where multiple copies or clones of the nucleic acid sequence will be replicated. The nucleic acid sequence may be of genomic, cDNA, RNA, semi-synthetic, synthetic origin, or any combinations thereof.

As used herein, two or more DNA coding sequences are said to be “joined” or “fused” when, as a result of in-frame fusions between the DNA coding sequences, the DNA coding sequences are translated into a fusion polypeptide. The term “fusion” in reference to Tf fusions includes, but is not limited to, attachment of at least one EPM peptide to the N-terminal end of Tf, attachment to the C-terminal end of Tf, and/or insertion between any two amino acids within Tf.

As used herein, “lubricants” are materials that perform a number of functions in tablet manufacture, such as improving the rate of flow of the tablet granulation, preventing adhesion of the tablet material to the surface of the dies and punches, reducing interparticle friction, and facilitating the ejection of the tablets from the die cavity. Commonly used lubricants include talc, magnesium stearate, calcium stearate, stearic acid, and hydrogenated vegetable oils. Typical amounts of lubricants range from about 0.1% by weight to about 5% by weight.

As used herein, the term “modification” or “modified” refers to an EPM peptide, which has the addition, deletion, or replacement of at least one amino acid of the EMP-1 amino acid sequence (i.e., SEQ ID NO: 4). In addition, “modification” or “modified” can refer to the addition of one or more linkers to the C-terminal, N-terminal, or any internal amino acid of the EMP-1 amino acid sequence. Examples of modifications to EMP-1 include, but are not limited to, deletion of one or more cysteine residues, replacement of one or more cysteine residues with an amino acid; or the addition of one or more linker groups to the C-terminal, N-terminal, or and internal amino acid. Further examples include replacement of certain hydrophobic residues with more hydrophilic residues (or less hydrophobic). For instance, Leu11 or Val14 may be changed to, for example, Glu, Asp, Lys, Arg, His, Asn, Gln, Ser or Thr. Preferably, Leu11 is changed to Glu or Val14 is changed to Glu. Alternatively, Leu11 is changed to Thr and Val14 is changed to Asp. Modifications of EMP-1 peptides of the invention do not include deletion of amino acid 1-3 and 17-20 of EMP-1 (i.e., do not include EMP-20).

As used herein, “modified transferrin” refers to a transferrin molecule that exhibits at least one modification of its amino acid sequence, compared to wild-type transferrin.

As used herein, “modified transferrin fusion protein” refers to a protein formed by the fusion of at least one molecule of modified transferrin (or a fragment or variant thereof) to at least one molecule of EPM (or fragment or variant thereof).

As used herein the term “non-essential” amino acid residue refers to a residue that can be altered from the native sequence of EMP-1 without altering the biological activity, whereas an “essential” amino acid residue is required for biological activity (e.g., Tyr 4 and Trp13 of EMP-1).

As used herein, the terms “nucleic acid,” “nucleic acid molecule,” or “polynucleotide” refer to deoxyribonucleotides or ribonucleotides and polymers thereof in either single- or double-stranded form. Unless specifically limited, the terms encompass nucleic acids containing analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Cassol et al. (1992); Rossolini et al. (1994) Mol. Cell. Probes 8:91-98). The term nucleic acid is used interchangeably with gene, cDNA, and mRNA encoded by a gene. As used herein, the terms “nucleic acid,” “nucleic acid molecule,” or “polynucleotide” are intended to include DNA molecules (e.g., cDNA or genomic DNA), RNA molecules (e.g., mRNA), analogs of the DNA or RNA generated using nucleotide analogs, and derivatives, fragments and homologs thereof.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nucleotides in length, preferably about 15 nucleotides to 30 nucleotides in length. Oligonucleotides may be chemically synthesized and may be used as probes.

As used herein, a DNA segment is referred to as “operably linked” or “operatively linked” when it is placed into a functional relationship with another DNA segment. For example, DNA for a signal sequence is operably linked to DNA encoding a fusion protein of the invention if it is expressed as a preprotein that participates in the secretion of the fusion protein; a promoter or enhancer is operably linked to a coding sequence if it stimulates the transcription of the sequence. Generally, DNA sequences that are operably linked are contiguous, and in the case of a signal sequence or fusion protein both contiguous and in reading phase. However, enhancers need not be contiguous with the coding sequences whose transcription they control. Linking, in this context, is accomplished by ligation at convenient restriction sites or at adapters or linkers inserted in lieu thereof.

As used herein, “pharmaceutically acceptable” refers to materials and compositions that are physiologically tolerable and do not typically produce an allergic or similar untoward reaction, such as gastric upset, dizziness and the like, when administered to a human. Typically, as used herein, the term “pharmaceutically acceptable” means approved by a regulatory agency of the Federal or a state government or listed in the U.S. Pharmacopeia or other generally recognized pharmacopeia for use in animals, and more particularly in humans.

As used herein and unless otherwise indicated, the term “pharmaceutically acceptable clathrate” means an EPM peptide or a fusion protein of the invention that is in the form of a crystal lattice that contains spaces (e.g., channels) that have a guest molecule (e.g., a solvent or water) trapped within.

As used herein and unless otherwise indicated, the term “pharmaceutically acceptable hydrate” means an EPM peptide or a fusion protein of the invention that further includes a stoichiometric or non-stoichiometric amount of water bound by non-covalent intermolecular forces.

As used herein and unless otherwise indicated, the term “pharmaceutically acceptable polymorph” refers to an EPM peptide or a fusion protein of the invention that exists in several distinct forms (e.g., crystalline, amorphous), the invention encompasses all of these forms. Polymorphs are, by definition, crystals of the same molecule having different physical properties as a result of the order of the molecules in the crystal lattice. The differences in physical properties exhibited by polymorphs affect pharmaceutical parameters such as storage stability, compressibility and density (important in formulation and product manufacturing), and dissolution rates (an important factor in determining bio-availability). Differences in stability can result from changes in chemical reactivity (e.g., differential oxidation, such that a dosage form discolors more rapidly when comprised of one polymorph than when comprised of another polymorph) or mechanical changes (e.g., tablets crumble on storage as a kinetically favored polymorph converts to thermodynamically more stable polymorph) or both (e.g., tablets of one polymorph are more susceptible to breakdown at high humidity). As a result of solubility/dissolution differences, in the extreme case, some polymorphic transitions may result in lack of potency or, at the other extreme, toxicity. In addition, the physical properties of the crystal may be important in processing: for example, one polymorph might be more likely to form solvates or might be difficult to filter and wash free of impurities (i.e., particle shape and size distribution might be different between one polymorph relative to the other).

As used herein and unless otherwise indicated, the term “pharmaceutically acceptable prodrug” means a derivative of an EPM peptide or a fusion protein that can hydrolyze, oxidize, or otherwise react under biological conditions (in vitro or in vivo) to provide the compound. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise oligonucleotides, peptides, lipids, aliphatic and aromatic groups, or NO, NO₂, ONO, and ONO₂ moieties. Prodrugs can typically be prepared using well known methods, such as those described in Burger's Medicinal Chemistry and Drug Discovery, 172 178, 949 982 (Manfred E. Wolff ed., 5th ed. 1995), and Design of Prodrugs (H. Bundgaard ed., Elselvier, New York 1985).

As used herein and unless otherwise indicated, the phrase “pharmaceutically acceptable salt(s),” includes, but is not limited to, salts of acidic or basic groups that may be present in an EPM peptide or a fusion protein used in the present compositions. EPM peptides or fusion proteins included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, (i.e., salts containing pharmacologically acceptable anions), including, but not limited to, sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. EPM peptides or fusion proteins included in the present compositions that include an amino moiety may form pharmaceutically acceptable salts with various amino acids, in addition to the acids mentioned above. EPM peptides or fusion proteins thereof, included in the present compositions, that are acidic in nature are capable of forming base salts with various pharmacologically acceptable cations. Examples of such salts include alkali metal or alkaline earth metal salts and, particularly, calcium, magnesium, sodium lithium, zinc, potassium, and iron salts.

As used herein and unless otherwise indicated, the term “pharmaceutically acceptable solvate” means an EPM peptide or a fusion protein of the invention that further includes a stoichiometric or non-stoichiometric amount of a solvent bound by non-covalent intermolecular forces. Preferred solvents are volatile, non-toxic, and/or acceptable for ai stration to humans in trace amounts.

As used herein, “physiologically effective amount” is that amount delivered to a subject to give the desired palliative or curative effect. This amount is specific for each drug and its ultimate approved dosage level.

As used herein, the term “powder” means a composition that consists of finely dispersed solid particles that are free flowing and capable of being readily dispersed in an inhalation device and subsequently inhaled by a subject so that the particles reach the lungs to permit penetration into the alveoli. Thus, the powder is said to be “respirable.” Preferably the average particle size is less than about 10 microns (μm) in diameter with a relatively uniform spheroidal shape distribution. More preferably the diameter is less than about 7.5 μm and most preferably less than about 5.0 μm. Usually the particle size distribution is between about 0.1 μm and about 5 μm in diameter, particularly about 0.3 μm to about 5 μm.

“Probes” refer to nucleic acid sequences of variable length, preferably between at least about 10 nucleotides (nt), 100 nt, or as many as about, for example, 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

As used herein, the term “promoter” refers to a region of DNA involved in binding RNA polymerase to initiate transcription.

As used herein and unless otherwise indicated, the term “prophylactically effective” refers to an amount of an EPM peptide or a fusion protein thereof or a pharmaceutically acceptable salt, solvate, hydrate, clathrate, polymorph, or prodrug thereof causing a reduction of the risk of acquiring a given disease or disorder. In one embodiment, the compositions of the invention are administered as a preventative measure to an animal, preferably a human, having a genetic predisposition to a disorder described herein. In another embodiment of the invention, the EPM peptide or a fusion protein thereof or compositions comprising an EPM peptide or a fusion protein thereof are administered as a preventative measure to a patient having a non-genetic predisposition to a disorder disclosed herein. Accordingly, the compositions of the invention may be used for the prevention of one disease or disorder and concurrently treating another (e.g., prevention of benign prostatic hyperplasia, while treating urinary incontinence).

As used herein, the term “recombinant” refers to a cell, tissue or organism that has undergone transformation with a new combination of genes or DNA.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide. Stringent conditions are known to those skilled in the art and can be found in Ausubel et al., (eds.), Current Protocols in Molecular Biology, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C.

As used herein, the term “substantially reduces” refers to the ability an EPM peptide to form a disulfide bond. The reduction in disulfide bond formation may be exhibited as a reduction in receptor binding as determined by biological assays, for example, as set forth in U.S. Pat. No. 5,773,569, which is incorporated herein by reference in its entirety. Other biological assays that can be used to demonstrate the activity of the compounds of the invention are disclosed in Greenberger et al. (1983) Proc. Natl. Acad. Sci. USA 80:2931-2935 (EPO-dependent hematopoietic progenitor cell line); Quelle and Wojchowski (1991) J. Biol. Chem. 266:609-614 (protein tyrosine phosphorylation in B6SUt.EP cells); Dusanter-Fourt et al. (1992) J. Biol. Chem. 287:10670-10678 (tyrosine phosphorylation of EPO-receptor in human EPO-responsive cells); Quelle et al. (1992) J. Biol. Chem. 267:17055-17060 (tyrosine phosphorylation of a cytosolic protein (pp 100) in FDC-ER cells); Worthington et al. (1987) Exp. Hematol. 15:85-92 (colorimetric assay for hemoglobin); Kaiho and Miuno (1985) Anal. Biochem. 149:117-120 (detection of hemoglobin with 2,7-diaminofluorene); Patel et al. (1992) J. Biol. Chem. 267:21300-21302 (expression of c-myb; Witthuhn et al. (1993) Cell 74:227-236 (association and tyrosine phosphorylation of JAK2); Leonard et al. (1993) Blood 82:1071-1079 (expression of GATA transcription factors); Ando et al. (1993) Proc. Natl. Acad. Sci. USA 90:9571-9575 (regulation of G₁/3 transition by cycling D2 and D3); and calcium flux, each of which is incorporated herein by reference.

As used herein, the term “subject” can be a human, a mammal, or an animal. The subject being treated is a patient in need of treatment.

As used herein, a targeting entity, protein, polypeptide or peptide refers to a molecule that binds specifically to a particular cell type [normal (e.g., lymphocytes) or abnormal (e.g., cancer cell)] and therefore may be used to target a Tf fusion protein or compound (drug, or cytotoxic agent) to that cell type specifically.

As used herein, “tablets” are solid pharmaceutical dosage forms containing drug substances with or without suitable diluents and prepared either by compression or molding methods well known in the art. Tablets have been in widespread use since the latter part of the 19^{th} century and their popularity continues. Tablets remain popular as a dosage form because of the advantages afforded both to the manufacturer (e.g., simplicity and economy of preparation, stability, and convenience in packaging, shipping, and dispensing) and the patient (e.g., accuracy of dosage, compactness, portability, blandness of taste, and ease of administration). Although tablets are most frequently discoid in shape, they may also be round, oval, oblong, cylindrical, or triangular. They may differ greatly in size and weight depending on the amount of drug substance present and the intended method of administration. They are divided into two general classes, (1) compressed tablets, and (2) molded tablets or tablet triturates. In addition to the active or therapeutic ingredient or ingredients (i.e., an EPM peptide or a fragment thereof), tablets contain a number or inert materials or additives. A first group of such additives includes those materials that help to impart satisfactory compression characteristics to the formulation, including diluents, binders, and lubricants. A second group of such additives helps to give additional desirable physical characteristics to the finished tablet, such as disintegrators, colors, flavors, and sweetening agents.

As used herein and unless otherwise indicated, the term “therapeutically effective” refers to an amount of an EPM peptide or fusion protein of the invention or a pharmaceutically acceptable salt, solvate, hydrate, clathrate, polymorph, or prodrug thereof able to cause an amelioration of a disease or disorder, or at least one discernible symptom thereof. “Therapeutically effective” refers to an amount of an EPM peptide or fusion protein of the invention or a pharmaceutically acceptable salt, solvate, hydrate, clathrate, polymorph, or prodrug thereof to result in an amelioration of at least one measurable physical parameter, not necessarily discernible by the patient. In yet another embodiment, the term “therapeutically effective” refers to an amount of an EPM peptide or fusion protein or a pharmaceutically acceptable salt, solvate, hydrate, clathrate, polymorph, or prodrug thereof to inhibit the progression of a disease or disorder, either physically (e.g., stabilization of a discernible symptom), physiologically (e.g., stabilization of a physical parameter), or both. In yet another embodiment, the term “therapeutically effective” refers to an amount of an EPM peptide or fusion protein or a pharmaceutically acceptable salt, solvate, hydrate, clathrate, polymorph, or prodrug thereof resulting in a delayed onset of a disease or disorder. The amount of fusion protein, which constitutes a “therapeutically effective amount” will vary depending on the EPM peptide used, the severity of the condition or disease, and the age and body weight of the subject to be treated, but can be determined routinely by one of ordinary skill in the art having regard to his/her own knowledge and to this disclosure.

As used herein, “therapeutic protein” refers to EPM peptides or fragments or variants thereof, having one or more therapeutic and/or biological activities. The terms peptides, proteins, and polypeptides are used interchangeably herein. Additionally, the term “therapeutic protein” may refer to the endogenous or naturally occurring correlate of an EPM protein. By a polypeptide displaying a “therapeutic activity” or a protein that is “therapeutically active” is meant an EPM peptide that possesses one or more known biological and/or therapeutic activities associated with EMP-1 or EPO. As a non-limiting example, a “therapeutic protein” is an EPM peptide that is useful to treat, prevent or ameliorate a disease, condition or disorder. Such a disease, condition or disorder may be in humans or in a non-human animal, e.g., veterinary use.

As used herein, the term “transformation” refers to the transfer of nucleic acid (i.e., a nucleotide polymer) into a cell. As used herein, the term “genetic transformation” refers to the transfer and incorporation of DNA, especially recombinant DNA, into a cell.

As used herein, the term “transformant” refers to a cell, tissue or organism that has undergone transformation.

As used herein, the term “transgene” refers to a nucleic acid that is inserted into an organism, host cell or vector in a manner that ensures its function.

As used herein, the term “transgenic” refers to cells, cell cultures, organisms, bacteria, fungi, animals, plants, and progeny of any of the preceding, which have received a foreign or modified gene and in particular a gene encoding a modified Tf fusion protein by one of the various methods of transformation, wherein the foreign or modified gene is from the same or different species than the species of the organism receiving the foreign or modified gene.

“Variants or variant” refers to a polynucleotide or nucleic acid differing from a reference nucleic acid or polypeptide, but retaining essential properties thereof. Generally, variants are overall closely similar, and, in many regions, identical to the reference nucleic acid or polypeptide. As used herein, “variant” refers to an EPM portion of a transferrin fusion protein of the invention, differing in sequence from a native EMP-1 but retaining at least one functional and/or therapeutic property thereof as described elsewhere herein or otherwise known in the art.

As used herein, the term “vector” refers broadly to any plasmid, phagemid or virus encoding an exogenous nucleic acid. The term is also be construed to include non-plasmid, non-phagemid and non-viral compounds which facilitate the transfer of nucleic acid into virions or cells, such as, for example, polylysine compounds and the like. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746). Examples of viral vectors include, but are not limited to, a recombinant vaccinia virus, a recombinant adenovirus, a recombinant retrovirus, a recombinant adeno-associated virus, a recombinant avian pox virus, and the like (Cranage et al., 1986, EMBO J. 5:3057-3063; International Patent Application No. WO 94/17810, published Aug. 18, 1994; International Patent Application No. WO 94/23744, published Oct. 27, 1994). Examples of non-viral vectors include, but are not limited to, liposomes, polyamine derivatives of DNA, and the like.

As used herein, the term “wild type” refers to a polynucleotide or polypeptide sequence that is naturally occurring.

As used herein, the terms “wild-type EMP-1” and “native EMP-1” are used synonymously and refer to EMP-1 having the following sequence:

SEQ ID NO: 4

GGTYSCHFGPLTWVCKPQGG,.

General Description

The invention encompasses EPM peptides or pharmaceutically acceptable salts, solvates, hydrates, clathrates, polymorphs, or prodrugs thereof. In a particular embodiment, the EPM peptides have extended serum stability or increased in vivo half-life compared to EMP-1. The EPM peptides of the invention have been modified by the deletion, addition, or replacement of at least one amino acid of the EMP-1 peptide sequence and optionally through the addition of at least one linker group to the C-terminal, N-terminal or an internal amino acid. In a particular embodiment, the EPM peptides of the invention retain their structure, activity, and function compared to EMP-1 and preferably have increased serum stability or increased in vivo half-life compared to EMP-1.

In one embodiment, the EPM peptide comprises a first modification of at least one cysteine residue that substantially reduces disulfide bond formation and a second modification such that the peptide exhibits EMP-1 activity. In a particular embodiment, the first modification comprises deleting at least one cysteine from the EMP-1 amino acid sequence, while maintaining the structure, activity, and function of the EMP-1 peptide. In another particular embodiment, the first modification comprises the replacement of at least one cysteine with any one of the following amino acids: arginine, asparagine, aspartic acid, glutamine, glutamic acid, gamma carboxyl glutamic acid, histidine, lysine, methionine, proline, serine, threonine, tryptophan, or tyrosine. In another particular embodiment, the first modification comprises the conservative substitution of at least one cysteine. In another particular embodiment, the first modification comprises the conservative substitution of at least one cysteine with an asparagine, glutamine, serine, threonine, or tyrosine. In another particular embodiment, the first modification comprises the substitution of at least one cysteine with aspartic acid or gamma carboxyl glutamic acid. In another particular embodiment, the substitution of at least one cysteine residue allows circularization to the peptide.

The invention also encompasses EPM peptides comprising a second modification comprising the addition of at least one linker group to an EMP-1 peptide. In one embodiment, the linker is covalently bonded to the C-terminal amino acid of an EMP-1 peptide. In another embodiment, the linker is covalently bonded to the N-terminal amino acid of an EMP-1 peptide. In yet another embodiment, the linker is covalently bonded to an internal amino acid of an EMP-1 peptide. In still another embodiment, the linker is covalently bonded to the N-terminal amino acid and the C-terminal amino acid of an EMP-1 peptide. In a particular embodiment, the linker is an amino acid linker. Linkers of the invention are described in more detail below.

In another embodiment, the first modification reduces binding of the peptide to the erythropoietin receptor in the absence of the second modification. In another embodiment, the second modification restores detectable binding of the peptide to the erythropoietin receptor.

In another embodiment, the invention encompasses pharmaceutical compositions comprising a therapeutically or prophylactically effective amount of at least one EPM peptide or a pharmaceutically acceptable salt, solvate, hydrate, clathrate, polymorph, or prodrug thereof, which is useful in treating or preventing disorders and disease states of hematological irregularity.

In another embodiment, the invention encompasses pharmaceutical compositions comprising a therapeutically or prophylactically effective amount of at least one modified EPM peptide or a pharmaceutically acceptable salt, solvate, hydrate, clathrate, polymorph, or prodrug thereof. The pharmaceutical compositions are useful in treating or preventing disorders including, but not limited to, anemia, beta-thalassemia, cystic fibrosis, pregnancy and menstrual disorders, early anemia of prematurity, spinal cord injury, acute blood loss, aging, neoplastic disease states associated with abnormal erythropoiesis, and renal insufficiency. The pharmaceutical compositions are also useful in treating or preventing chronic or recurrent diseases include, but are not limited to, viral disease or infections, cancer, a metabolic diseases, obesity, autoimmune diseases, inflammatory diseases, allergy, graft-vs.-host disease, systemic microbial infection, cardiovascular disease, psychosis, genetic diseases, neurodegenerative diseases, disorders of hematopoietic cells, diseases of the endocrine system or reproductive systems, gastrointestinal diseases. Further illustrative examples of these classes of disease include, but are not limited to, diabetes, multiple sclerosis, asthma, HCV or HIV infections, hypertension, hypercholesterolemia, arterial scherosis, arthritis, and Alzheimer's disease.

In another embodiment, the invention encompasses a method of treating or preventing a disorder that can be treated or prevented by stimulating or regulating the production of erythrocytes, which comprises administering to a patient in need thereof a therapeutically or prophylactically effective amount of at least one EPM peptide or a pharmaceutically acceptable salt, solvate, hydrate, clathrate, polymorph, or prodrug thereof.

In another embodiment, the invention encompasses a method of treating or preventing anemia, beta-thalassemia, cystic fibrosis, pregnancy and menstrual disorders, early anemia of prematurity, spinal cord injury, acute blood loss, aging, neoplastic disease states associated with abnormal erythropoiesis, and renal insufficiency, which comprises administering to a patient in need thereof a therapeutically or prophylactically effective amount of at least one EPM peptide or a pharmaceutically acceptable salt, solvate, hydrate, clathrate, polymorph, or prodrug thereof.

In another embodiment, the invention encompasses a method of treating or preventing chronic or recurrent diseases include, but are not limited to, viral disease or infections, cancer, a metabolic diseases, obesity, autoimmune diseases, inflammatory diseases, allergy, graft-vs.-host disease, systemic microbial infection, cardiovascular disease, psychosis, genetic diseases, neurodegenerative diseases, disorders of hematopoietic cells, diseases of the endocrine system or reproductive systems, gastrointestinal diseases, diabetes, multiple sclerosis, asthma, HCV or HIV infections, hypertension, hypercholesterolemia, arterial scherosis, arthritis, or Alzheimer's disease, which comprises administering to a patient in need thereof a therapeutically or prophylactically effective amount of at least one EPM peptide or a pharmaceutically acceptable salt, solvate, hydrate, clathrate, polymorph, or prodrug thereof.

In another embodiment, the invention encompasses one or more EPM peptides fused to a second peptide or protein to generate a “fusion protein.” In a particular embodiment, the fusion proteins have extended serum stability or increased in vivo half-life compared to EMP-1. In a particular embodiment, the EPM peptide is fused to the C-Terminal end of the second peptide or protein. In another particular embodiment, the EPM peptide is fused to the N-Terminal end of the second peptide or protein. In another particular embodiment, the EPM peptide is inserted into at least one loop of a second peptide or protein. In another particular embodiment, the fusion protein comprises a portion of the N domain of a second peptide or protein, a bridging peptide and a portion of the C domain of a second peptide or protein, wherein the bridging peptide links the EPM peptide to a second peptide or protein. In another particular embodiment, the fusion protein comprises an EPM peptide that is inserted between an N and a C domain of the second peptide or protein. In another particular embodiment, the second peptide or protein comprises a hinge region wherein at least one amino acid substitution, deletion or addition in the hinge region. In another particular embodiment, the second peptide or protein comprises at least one loop, and the EPM peptide replaces at least one loop of a second peptide or protein.

In another embodiment, the invention encompasses pharmaceutical compositions comprising a therapeutically or prophylactically effective amount of at least one fusion protein or a pharmaceutically acceptable salt, solvate, hydrate, clathrate, polymorph, or prodrug thereof, which is useful in treating or preventing disorders and disease states of hematological irregularity.

In another embodiment, the invention encompasses pharmaceutical compositions comprising a therapeutically or prophylactically effective amount of at least one fusion protein or a pharmaceutically acceptable salt, solvate, hydrate, clathrate, polymorph, or prodrug thereof. These pharmaceutical compositions are useful in treating or preventing disorders including, but not limited to, anemia, beta-thalassemia, cystic fibrosis, pregnancy and menstrual disorders, early anemia of prematurity, spinal cord injury, acute blood loss, aging, neoplastic disease states associated with abnormal erythropoiesis, and renal insufficiency. The pharmaceutical compositions are also useful in treating or preventing chronic or recurrent diseases include, but are not limited to, viral disease or infections, cancer, a metabolic diseases, obesity, autoimmune diseases, inflammatory diseases, allergy, graft-vs.-host disease, systemic microbial infection, cardiovascular disease, psychosis, genetic diseases, neurodegenerative diseases, disorders of hematopoietic cells, diseases of the endocrine system or reproductive systems, gastrointestinal diseases. Further illustrative examples of these classes of disease include, but are not limited to, diabetes, multiple sclerosis, asthma, HCV or HIV infections, hypertension, hypercholesterolemia, arterial scherosis, arthritis, and Alzheimer's disease.

In another embodiment, the invention encompasses a method of treating or preventing a disorder that can be treated or prevented by stimulating or regulating the production of erythrocytes, which comprises administering to a patient in need thereof a therapeutically or prophylactically effective amount of at least one fusion protein or a pharmaceutically acceptable salt, solvate, hydrate, clathrate, polymorph, or prodrug thereof.

In another embodiment, the invention encompasses a method of treating or preventing anemia, beta-thalassemia, cystic fibrosis, pregnancy and menstrual disorders, early anemia of prematurity, spinal cord injury, acute blood loss, aging, neoplastic disease states associated with abnormal erythropoiesis, and renal insufficiency, which comprises administering to a patient in need thereof a therapeutically or prophylactically effective amount of at least one fusion protein or a pharmaceutically acceptable salt, solvate, hydrate, clathrate, polymorph, or prodrug thereof.

In another embodiment, the invention encompasses a method of treating or preventing chronic or recurrent diseases include, but are not limited to, viral disease or infections, cancer, a metabolic diseases, obesity, autoimmune diseases, inflammatory diseases, allergy, graft-vs.-host disease, systemic microbial infection, cardiovascular disease, psychosis, genetic diseases, neurodegenerative diseases, disorders of hematopoietic cells, diseases of the endocrine system or reproductive systems, gastrointestinal diseases, diabetes, multiple sclerosis, asthma, HCV or HIV infections, hypertension, hypercholesterolemia, arterial scherosis, arthritis, or Alzheimer's disease, which comprises administering to a patient in need thereof a therapeutically or prophylactically effective amount of at least one fusion protein or a pharmaceutically acceptable salt, solvate, hydrate, clathrate, polymorph, or prodrug thereof.

In another embodiment, the invention encompasses kits containing one or more EPM peptides or fusion proteins, which can be used, for instance, for the therapeutic or non-therapeutic applications, which further comprises a container with a label.

EPM Peptides

The invention encompasses modifications or variants of the EMP-1 peptide (also referred to as the “EMP-1 protein”) that function as erythropoietin mimetic peptides (referred to herein as “EPM,” “EPM peptide(s),” or “EPM protein(s)” see e.g. U.S. Pat. No. 5,773,569). For instance, an EMP-1 peptide may comprise a sequence of 10 to 40 amino acid residues in length that binds to erythropoietin receptor and comprises a sequence of amino acids X₃ X₄ X₅ G P X₆ T W X₇ X₈ (SEQ ID NO: 31) where each amino acid is indicated by standard one letter abbreviation; X₆ is independently selected from any one of the 20 genetically coded L-amino acids; X₃ is C; X₄ is R, H, L, or W; X₅ is M, F, or I; X₇ is D, E, I, L, or V; and X₈ is C. An EPM peptide can retain substantially the same, or a subset of, the biological activities of the EMP-1 protein. Thus, specific biological effects can be elicited by treatment with an EPM peptide of limited function. In one embodiment, treatment of a subject with an EPM peptide having a subset of the biological activities of the native form of the EMP-1 peptide has fewer side effects in a subject relative to treatment with the native form of the EMP-1 peptide.

EPM peptides or proteins of the invention comprise a first modification of at least one cysteine residue that substantially reduces disulfide bond formation and a second modification such that the EPM peptide exhibits EMP-1 activity or functions as an erythropoietin mimetic. In some embodiments, the first and second modifications may be the same or a single modification such that the modification substantially reduces cysteine disulfide bond formation and the EPM peptide still exhibits EMP-1 activity.

An EPM peptide of the invention that preserves EMP-1-like function includes any modification in which residues at a particular position in the sequence have been substituted by other amino acids and further includes the possibility of inserting an additional residue or residues between two residues of the native EMP-1 peptide as well as the possibility of deleting one or more residues from the native EMP-1 peptide sequence. Any amino acid substitution, insertion, or deletion is encompassed by the invention. In favorable circumstances, the substitution is a conservative substitution as defined herein.

The modifications or variants of the EMP-1 sequence may be introduced by mutation into at least one position of the EMP-1 nucleotide sequence thereby leading to changes in the amino acid sequence of the encoded EPM peptide, without altering the functional ability of the EMP-1 peptide. For example, nucleotide substitutions leading to amino acid substitutions at non-essential amino acid residues can be made in the sequence of SEQ ID NO: 1 or the EMP-1 nucleotide sequence of a DNA insert of a plasmid or vector known in the art.

In a particular embodiment, the modification is such that the structure, activity, and function of the EPM peptide are retained compared to EMP-1. Examples of retention of structure, activity, and function include, but are not limited to, stabilization of the β-sheet structure relative to the EMP-1 peptide, competitive receptor binding assays compared to EMP-1 peptide, and similar cascade of phosphorylation events and cell cycle progression in EPO responsive cells. In one embodiment, the EPM peptide encompasses deletion of one or more amino acid residues such that the EPM peptide retains structure, activity, and function. In another embodiment, the EPM peptide encompasses replacement of one or more amino acid residues with an amino acid that will allow the EPM peptide to retain structure, activity, and function. In another embodiment, the EPM peptide encompasses the addition of one or more amino acids to the C-terminal or N-terminal amino acids or both or an internal amino acid. In another embodiment, the EPM peptide encompasses the addition of one or more linker groups to the C-terminal, N-terminal, or an internal amino acid. Another embodiment of the invention encompasses an EPM peptide that has been modified by the deletion, addition or replacement of at least one amino acid of the EPM peptide sequence and through the addition of at least one linker group to the C-terminal or N-terminal amino acids or both, wherein the EPM peptide retains or increases its structure, activity, and function compared to an EMP-1 peptide. A further embodiment of the invention encompasses an EPM peptide in which the amino acid sequence is reversed with respect to that in EMP-1.

EMP-1 normally circularizes through cysteine-cysteine bonds to form disulfides. In one embodiment, the EPM peptide comprises a first modification of at least one cysteine residue that substantially reduces disulfide bond formation and a second modification such that the peptide exhibits EMP-1 activity. In a particular embodiment, the invention encompasses a first modification comprising replacement of one or more cysteine residues of EMP-1 with an amino acid that results in substantially reduced disulfide bond formation, while preserving or maintaining circularization or cyclization of the EPM and retaining or increasing the therapeutic or prophylactic activity compared to EMP-1. An illustrative amino acid capable of replacing a cysteine residue while restoring circularization of the EPM peptide includes, but is not limited to, aspartic acid to form a lactone or lactam. In another particular embodiment, a first modification comprises deleting one or more cysteine residues from EMP-1 resulting in substantially reduced disulfide bond formation, while retaining or increasing the therapeutic or prophylactic activity of EMP-1 with a second modification that induces peptide cyclization.

In addition to the foregoing cyclization strategies, other non-disulfide peptide cyclization strategies can be employed. Such alternative cyclization strategies include, for example, amide-cyclization strategies as well as cyclization strategies involving the formation of thio-ether bonds. Thus, the compounds of the invention can exist in a cyclized form using, for example, an intramolecular amide bond or an intramolecular thio-ether bond.

In another embodiment, the invention encompasses an EPM peptide, wherein one or more cysteine residues is deleted from EMP-1 and comprises a second modification, wherein a linker is added to the C-terminus and/or N-terminus amino acid allowing circularization of the EPM peptide or induce peptide conformation requirement for EMP-1 activity, while retaining or increasing the therapeutic or prophylactic activity of EMP-1. In another embodiment, the invention encompasses an EPM peptide, wherein a linker is added to the C-terminus and/or N-terminus amino acid of EMP-1 allowing circularization to the EPM peptide while retaining or increasing the therapeutic or prophylactic activity of EMP-1. Exemplary linker groups include, but are not limited to, a molecule or group of molecules that connects two molecules, such as EPM and a second peptide or protein, and serves to place the two molecules in a preferred configuration so that the EPM peptide can bond to a second peptide or protein with minimal steric hindrance. For example, an EPM peptide to be fused to a second peptide or protein may be chemically cross-linked using linker molecules and linker molecule length optimization techniques known in the art. Thus, the second peptide or protein moiety and the EPM peptide of the fusion proteins of the invention can be fused directly or using a linker peptide of various lengths to provide greater physical separation and allow more spatial mobility between the fused proteins and thus maximize the accessibility of the EPM peptide, for instance, for binding to its cognate receptor. The linker peptide may consist of amino acids that are flexible or more rigid. For example, a linker such as, but not limited to, a poly-glycine stretch. The linker can be less than about 50, 40, 30, 20, or 10 amino acid residues. The linker can be covalently linked to and between the transferrin protein or portion thereof and the EPM peptide.

Modifications or variations can be introduced into the EMP-1 nucleotide sequence by standard techniques (e.g., site-directed mutagenesis and PCR-mediated mutagenesis). The techniques may be used to modify the one or more cysteine residues. In another embodiment, conservative amino acid substitutions are made at one or more predicted non-essential amino acid residues. Thus, a predicted nonessential amino acid residue in EMP-1 is replaced with another amino acid residue from the same side chain family. Alternatively, in another embodiment, mutations can be introduced randomly along all or part of an EMP-1 coding sequence (e.g., by saturation mutagenesis or discrete point mutation or truncation of the EMP-1 peptide), and the resultant mutants can be screened for EMP-1 biological activity to identify mutants that retain activity. Following mutagenesis, the EPM protein can be expressed by any recombinant technology known in the art and the activity of the protein can be determined. In addition, the EPM peptide may be generated using chemical techniques known in the art to form one or more inter-molecule cross-links between the amino acid residues located within the polypeptide sequence of the EMP-1. Alternatively, EPM peptides of the invention may be generated using genetic engineering techniques known in the art. In one embodiment, the EPM peptide contained in fusion protein of the invention is produced recombinantly using fusion protein technology described herein or otherwise known in the art.

The EPM peptides can be identified by screening combinatorial libraries of mutants (e.g., truncation mutants) of the EMP-1 peptide for EMP-1 peptide activity. In one embodiment, a variegated library of EPM peptides is generated by combinatorial mutagenesis at the nucleic acid level and is encoded by a variegated gene library. A variegated library of EPM peptides can be produced by, for example, enzymatically ligating a mixture of synthetic oligonucleotides into gene sequences such that a degenerate set of potential EPM peptide sequences is expressible as individual polypeptides, or alternatively, as a set of larger fusion proteins (e.g., for phage display) containing the set of EPM peptide sequences therein. There are a variety of methods, which can be used to produce libraries of potential EPM peptides from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential EPM peptide sequences. Methods for synthesizing degenerate oligonucleotides are known in the art (see, e.g., Narang (1983) Tetrahedron, 39:3; Itakura et al. (1984) Annu Rev Biochem, 53:323; Itakura et al. (1984) Science, 198:1056; Ike et al. (1983) Nucl Acid Res., 11:477.

In addition, the EPM peptide can be assayed for (1) the ability to form protein:protein interactions with a second peptide or protein, other cell-surface proteins, or biologically active portions thereof, (2) complex formation between an EPM peptide and an erythropoietin receptor; (3) the ability of an EPM peptide to bind to another protein.

Second Proteins and Peptides of the Invention

The invention encompasses fusion proteins that comprise one or more EPM peptides or a fragment thereof fused to or inserted in a second peptide or protein. Any second peptide or protein may be used to make fusion proteins of the invention. In one embodiment, a second peptide or protein refers to a peptide having an amino acid sequence corresponding to a protein that is substantially homologous to the EPM protein, (e.g., a protein that is the same as the EPM protein or that is derived from the same or a different organism). In another embodiment, a second peptide or protein refers to a peptide having an amino acid sequence corresponding to a protein that is not substantially homologous to the EPM protein, (e.g., a protein that is different from the EPM protein or that is derived from the same or a different organism).

In one embodiment, the second peptide or protein is one or more EPM peptides, thus creating a dimer, trimer, etc. In another embodiment, the second peptide or protein is not an EPM peptide. Illustrative second peptides or proteins capable of forming the fusion proteins of the invention include, but are not limited to, one or more EPM peptides, albumin, transferrin (“Tf”), melanotransferrin, lactotransferrin, IgG, an Fc fragment of IgG, maltose binding protein (“MBP”), green fluorescent protein (“GFP”), or glutathione S-transferase (“GST”).

However, fusion proteins of the invention may be generated with any second peptide or protein, or a fragment, domain, or engineered domain thereof. For instance, fusion proteins may be produced using a full-length second peptide or protein sequence (e.g. a Tf sequence), with or without the native second peptide or protein signal sequence (see e.g., U.S. application Ser. No. 10/231,494, filed Aug. 30, 2002, which is herein incorporated by reference in its entirety). Fusion proteins may also be made using a single second peptide or protein domain, such as an individual N or C domain or a modified form of a second peptide or protein comprising 2N or 2C domains (see, e.g., U.S. Provisional Application 60/406,977, filed Aug. 30, 2002, which is herein incorporated by reference in its entirety). In some embodiments, fusions of an EPM peptide to a single C domain may be produced, wherein the C domain is altered to reduce, inhibit or prevent glycosylation. In other embodiments, the use of a single N domain is advantageous as the second peptide or protein glycosylation sites reside in the C domain and the N domain, on its own. An illustrative embodiment of the fusion proteins of the invention having a single N domain which is expressed at a high level.

In another embodiment, the second peptide portion of the fusion protein of the invention includes a splice variant (e.g., a transferrin splice variant, a melanotransferrin splice variant, lactoferrin splice variant). In an illustrative embodiment, a splice variant can be a splice variant of human transferrin. In one specific embodiment, the human transferrin splice variant can be that of Genbank Accession AAA61140. In another illustrative embodiment, a second peptide splice variant can be a novel splice variant of a neutrophil lactoferrin. In a specific embodiment, the neutrophil lactoferrin splice variant can be that of Genbank Accession AAA59479. In another specific embodiment, the neutrophil lactoferrin splice variant can comprise the following amino acid sequence EDCIALKGEADA (SEQ ID NO: 5), which includes the novel region of splice-variance.

Linkers of the Invention

In one embodiment, the fusion protein includes a peptide linker and the peptide linker has one or more of the following characteristics: a) it ensures effective presentation of the peptide to solvent, in particular by providing spatial separation from the second protein; for example, it may allow for rotation of the EPM peptide amino acid sequence and the second peptide or protein amino acid sequence relative to each other; b) it is resistant to digestion where necessary by proteases; and c) it does not detrimentally interact with the EPM or the second peptide or protein.

In a preferred embodiment: the fusion protein includes a peptide linker and the peptide linker is 5 to 60, preferably, 10 to 30 amino acids in length. The peptide linker is 20 amino acids in length; the peptide linker is 17 amino acids in length; each of the amino acids in the peptide linker is Gly, Ser, Asn, Thr, or Ala; the peptide linker includes a Gly-Ser element.

In another preferred embodiment, the fusion protein includes a peptide linker and the peptide linker includes a sequence having the formula (Ser-Gly-Gly-Gly-Gly)_{y} (SEQ ID NO: 22) wherein y is 1, 2, 3, 4, 5, 6, 7, or 8. Preferably, the peptide linker includes a sequence having the formula (Ser-Gly-Gly-Gly-Gly)₃ (SEQ ID NO: 23). Preferably, the peptide linker includes a sequence having the formula ((Ser-Gly-Gly-Gly-Gly)₃-Ser-Pro) (SEQ ID NO: 24).

In a preferred embodiment, the fusion protein includes a peptide linker, and the peptide linker includes a sequence having the formula (Ser-Ser-Ser-Ser-Gly)_{y} (SEQ ID NO: 25) wherein y is 1, 2, 3, 4, 5, 6, 7, or 8. Preferably, the peptide linker includes a sequence having the formula ((Ser-Ser-Ser-Ser-Gly)₃-Ser-Pro) (SEQ ID NO: 26).

In a preferred embodiment, the fusion protein includes a peptide linker, and the peptide linker includes a sequence having the formula (Pro-Glu-Ala-Pro-Thr-Asp)_{y}, wherein y is 1, 2, 3, 4, 5, 6, 7, or 8 (SEQ ID NO: 32).

In a preferred embodiment, the fusion protein includes a peptide linker, and the peptide linker includes a sequence derived from the immunoglobulin hinge region which has the formula Ala-Glu-Pro-Lys-Ser-Cys-Glu-Lys-Thr-His-Thr-Cys-Pro-Pro-Cys-Pro-Ala-Pro-Glu-Leu-Leu-Gly-Gly-Pro-Ser (SEQ ID NO: 34). In a further preferred embodiment the Cys residues are changed to and other amino acid such as Ser.

In another preferred embodiment, the fusion protein includes a peptide linker, and the peptide linker is a polyglycine stretch.

EPM Peptide Fusion Proteins

The invention encompasses one or more EPM peptides fused to a second protein or peptide to generate a fusion protein, which possesses increased serum stability and increased in vivo circulatory half-life. Any EPM peptide sequence may be used to make an EPM peptide and therefore to make the fusion proteins of the invention. These sequences can then be inserted into a second protein or peptide loop to provide three-dimensional structure to the EPM region of the fusion protein. The invention encompasses the use of the fusion protein to treat various diseases and conditions associated with EPO such as, but not limited to, those described herein. In addition, the fusion proteins possess increased serum stability and increased in vivo circulatory half-life compared to an EMP-1 that is not fused to a second protein or peptide.

Any EPM peptide entity may be used as the fusion partner to a second peptide or protein according to the methods and compositions of the invention. The EPM peptide is typically a modification of EMP-1 (e.g., a C depletion or replacement) capable of exerting a beneficial biological effect in vitro or in vivo and includes proteins or peptides that exert a beneficial effect in relation to normal homeostasis, physiology or a disease state. For instance, a beneficial effect as related to a disease state includes any effect that is advantageous to the treated subject, including disease prevention, disease stabilization, the lessening or alleviation of disease symptoms or a modulation, alleviation or cure of the underlying defect to produce an effect beneficial to the treated subject.

In a particular embodiment, fusion protein of the invention includes at least a fragment or variant of an EPM protein and at least a fragment or variant of a second peptide or protein, which are associated with one another, for example, by genetic fusion.

The fusion proteins of the invention may contain one or more copies of the EPM peptide fused to a second peptide or protein. In a particular embodiment, an EPM fusion protein comprises at least one biologically active portion of an EPM peptide. In another particular embodiment, a fusion protein comprises at least two biologically active portions of an EPM peptide. In yet another embodiment, a fusion protein comprises at least three biologically active portions of an EPM peptide. The fusion of the EPM peptide may occur at any position of the second protein or peptide including, but not limited to, an internal position, the N-terminus, and/or the C-terminus of the second peptide or protein. In some embodiments, the EPM peptide is attached to both the N- and C-terminus of the second peptide or protein and the fusion protein may contain one or more equivalents of the EPM peptide on either or both ends of the second peptide or protein. In another embodiment, the EPM peptide is inserted into known domains of the second peptide or protein, for example, into one or more of the loops of the second peptide or protein. (See, e.g., Ali et al. (1999) J. Biol. Chem., 274(34): 24066-24073). In a particular embodiment, the EPM peptide may be inserted into all of the loops of the second peptide or protein to create a multivalent molecule with increased affinity for the receptor, or targeting molecule, which the EPM binds. In other embodiments, the EPM peptide is inserted between the N and C domains of the second peptide or protein. Alternatively, the EPM peptide is inserted or fused anywhere in the second peptide or protein.

In another embodiment, the fusion proteins contain an EPM peptide portion that can have one or more amino acids deleted from both the amino and the carboxy termini.

In another embodiment, the fusion protein contains an EPM peptide portion that is at least about 80%, 85%, 90%, identical to a reference EMP-1 set forth herein, or fragments thereof. In further embodiments, the fusion proteins contain an EPM peptide portion that is at least about 80%, 85%, 90% identical to reference EMP-1 having the amino acid sequence of N- and C-terminal deletions as described above. However, the EPM peptide is not EMP-20.

Even if deletion of one or more amino acids from the N-terminus of a protein results in modification or loss of one or more biological functions of the EPM peptide portion, other therapeutic activities and/or functional activities (e.g., biological activities, ability to multimerize, ability to bind a ligand) may still be retained. For example, the ability of an EPM peptide with N-terminal deletions to induce and/or bind to antibodies, which recognize the complete or mature forms of the EPM peptide generally will be retained with less than the majority of the residues of the native EMP-1 removed from the N-terminus. Whether a particular EPM peptide lacking N-terminal residues of a native EMP-1, retains such immunologic activities can be assayed by routine methods described herein and otherwise known in the art. It is not unlikely that a mutant with a large number of deleted N-terminal amino acid residues may retain some biological or immunogenic activities. In fact, peptides composed of as few as six amino acid residues may often evoke an immune response.

Also as mentioned above, even if deletion of one or more amino acids from the N-terminus or C-terminus of a EPM peptide results in modification or loss of one or more biological functions of the protein, other functional activities (e.g., biological activities, ability to multimerize, ability to bind a ligand) and/or therapeutic activities may still be retained. For example the ability of EPM peptide with C-terminal deletions to induce and/or bind to antibodies, which recognize the complete or mature forms of the EPM peptide generally will be retained when less than the majority of the residues of the complete or mature polypeptide are removed from the C-terminus. Whether a particular EPM peptide lacking the N-terminal and/or, C-terminal residues of a native EMP-1 retains therapeutic activity can readily be determined by routine methods described herein and/or otherwise known in the art.

Peptide fragments of the EPM peptide can be fragments comprising, or alternatively, consisting of, an amino acid sequence that displays a therapeutic activity and/or functional activity (e.g. biological activity) of the peptide sequence of the EPM peptide of which the amino acid sequence is a fragment.

The peptide fragments of the EPM peptide may comprise only the N- and C-termini of the protein, i.e., the central portion of the EPM peptide has been deleted. Alternatively, the EPM peptide fragments may comprise non-adjacent and/or adjacent portions of the central part of the EMP-1 peptide.

Generally, the fusion protein of the invention may have one second peptide or protein derived region and one EPM peptide region. Multiple regions of each protein, however, may be used to make a fusion protein of the invention. Similarly, more than one EPM peptide may be used to make a fusion protein of the invention, thereby producing a multi-functional modified fusion protein.

In another embodiment, the fusion protein of the invention contains an EPM peptide fused to the N terminus of a second peptide or protein. In an alternate embodiment, the fusion protein of the invention contains an EPM peptide fused to the C terminus of a second peptide or protein. In a further embodiment, the fusion protein of the invention contains a second peptide or protein fused to the N terminus of an EPM peptide. In an alternate embodiment, the fusion protein of the invention contains a second peptide or protein fused to the C terminus of an EPM peptide.

In another embodiment, the fusion protein of the invention contains an EPM peptide fused to both the N-terminus and the C-terminus of the second peptide or protein. In another embodiment, the N- and C-termini bind the same EPM peptide. In an alternate embodiment, the EPM peptides fused at the N- and C-termini are different EPM peptide entities. In another alternate embodiment, the EPM peptide fused to the N- and C-termini bind different EPM peptide entities, which may be used to treat or prevent the same disease, disorder, or condition. In another embodiment, the EPM peptide entities fused at the N- and C-termini are different EPM peptides, which may be used to treat or prevent different diseases or disorders, which are known in the art to commonly occur in patients simultaneously.

In addition to fusion proteins of the invention in which the EPM peptide portion is fused to the N terminal and/or C-terminal region of the second peptide or protein, fusion proteins of the invention may also be produced by inserting the EPM peptide (e.g., an EPM peptide as disclosed herein, or a fragment or variant thereof) into an internal region of the second peptide or protein. Internal regions of second peptide or protein include, but are not limited to, iron binding sites, hinge regions, bicarbonate binding sites, or receptor binding domain.

Within the protein sequence of the second peptide or protein a number of loops or turns may exist, which are stabilized by disulfide bonds. These loops are useful for the insertion, or internal fusion, of one or more EPM peptides or therapeutically active peptides particularly those requiring a secondary structure to be functional, or to generate a modified transferrin molecule with specific biological activity. In a particular embodiment, the C residues of the EPM peptide are substituted with a conservative amino acid substituent that can facilitate insertion or fusion into the second peptide or protein loop. In another particular embodiment, the C residues of the EPM peptide are substituted with a conservative amino acid substituent and a linker is added that can facilitate insertion or fusion into the second peptide or protein loop. In another particular embodiment, the C residues of the EPM peptides are preserved and a linker is added that can facilitate insertion or fusion into the second peptide or protein loop. In addition, where the C-terminus or N-terminus of a second peptide or protein appears to be more buried and secured by, for example, a disulfide bond, a linker or spacer moiety at the C-terminus or N-terminus may be used in some embodiments of the invention such as, for example, a poly-glycine stretch, to separate the EPM peptide from the second peptide or protein. In another embodiment, the C-terminal or N-terminal disulfide bond may be eliminated to untether the C-terminus or N-terminus.

When EPM peptide entities are inserted into or replace at least one loop of a second peptide or protein (e.g., a Tf molecule), insertions may be made within any of the surface exposed loop regions, in addition to other areas of the second peptide or protein. For example, insertions may be made within the loops comprising Tf amino acids 32-33, 74-75, 256-257, 279-280 and 288-289 (Ali et al., supra). As previously described, insertions may also be made within other regions of a second peptide or protein such as the sites for iron and bicarbonate binding, hinge regions, and the receptor binding domain as described herein. The loops in the second peptide or protein sequence that are amenable to modification/replacement for the insertion of EPM peptides may also be used for the development of a screenable library of random peptide inserts. Any procedures may be used to produce nucleic acid inserts for the generation of peptide libraries, including available phage and bacterial display systems, prior to cloning into a second peptide or protein domain and/or fusion to the ends of a second peptide or protein.

Where the C-terminus or N-terminus of a second peptide or protein is free and points away from the body of the molecule fusions of an EPM peptide on the C-terminus or N-terminus may be a preferred embodiment. Such fusions may include a linker region such as, but not limited to, a poly-glycine stretch, to separate the EPM peptide from the second peptide or protein.

For example, in one embodiment a protein comprises an EPM peptide domain operably linked to the extracellular domain of a second protein known to be involved in an activity of interest. Such fusion proteins can be further utilized in screening assays for compounds that modulate EPM peptide activity.

In another embodiment, the fusion protein is an EPM peptide containing a heterologous signal sequence at its N-terminus. In certain host cells (e.g., mammalian host cells), expression and/or secretion of an EPM peptide can be increased through use of a heterologous signal sequence.

An EPM peptide fusion protein of the invention can be produced by standard recombinant DNA techniques. For example, DNA fragments coding for the different EPM peptide sequences are ligated together in-frame in accordance with conventional techniques, (e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation). In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence. See, e.g., Ausubel et al. (eds.) Current Protocols in Molecular Biology, John Wiley & Sons, 1992). Moreover, many expression vectors are commercially available that already encode a fusion protein (e.g., a GST polypeptide); An EPM peptide-encoding nucleic acid can be cloned into such an expression vector such that the second peptide or protein is linked in-frame to the EPM peptide.

EPM Peptide/Transferrin Fusion Proteins

In an illustrative embodiment of the invention, the fusion protein includes a human transferrin (“Tf”), although any animal Tf molecule may be used to produce the fusion proteins of the invention, including human Tf variants, cow, pig, sheep, dog, rabbit, rat, mouse, hamster, echnida, platypus, chicken, frog, homworm, monkey, as well as other bovine, canine and avian species. All of these Tf sequences are readily available in GenBank and other public databases. The human Tf nucleotide sequence is available (see SEQ ID NOS 1, 2 and 3 and the accession numbers described above and available at www.ncbi.nlm.nih.gov/) and can be used to make genetic fusions between Tf or a domain of Tf and the EPM peptide. Fusions may also be made from related molecules such as lacto transferrin (lactoferrin) GenBank Acc: NM_{—}002343) or melanotransferrin (GenBank Acc. NM_{—}013900, murine melanotransferrin).

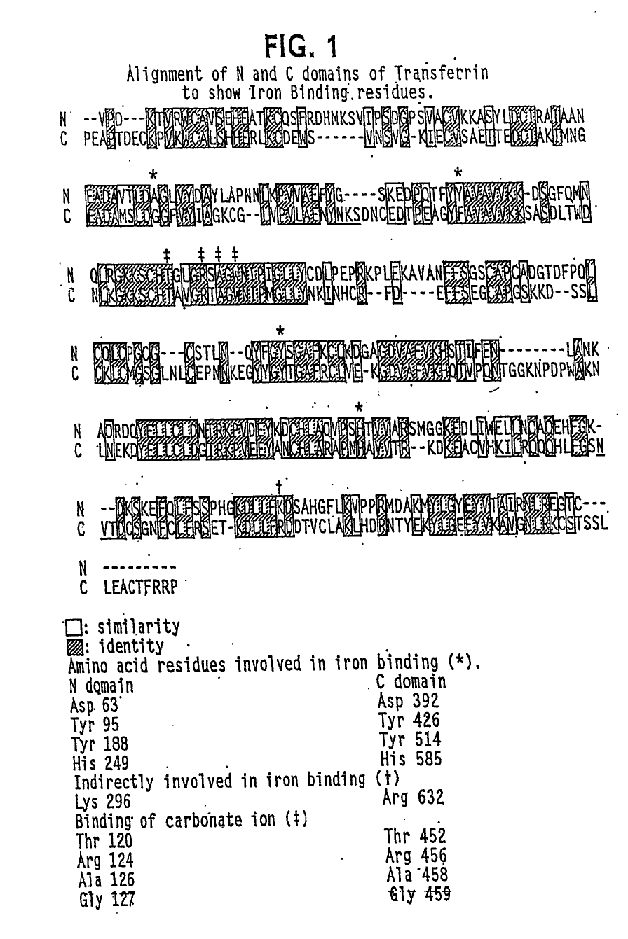

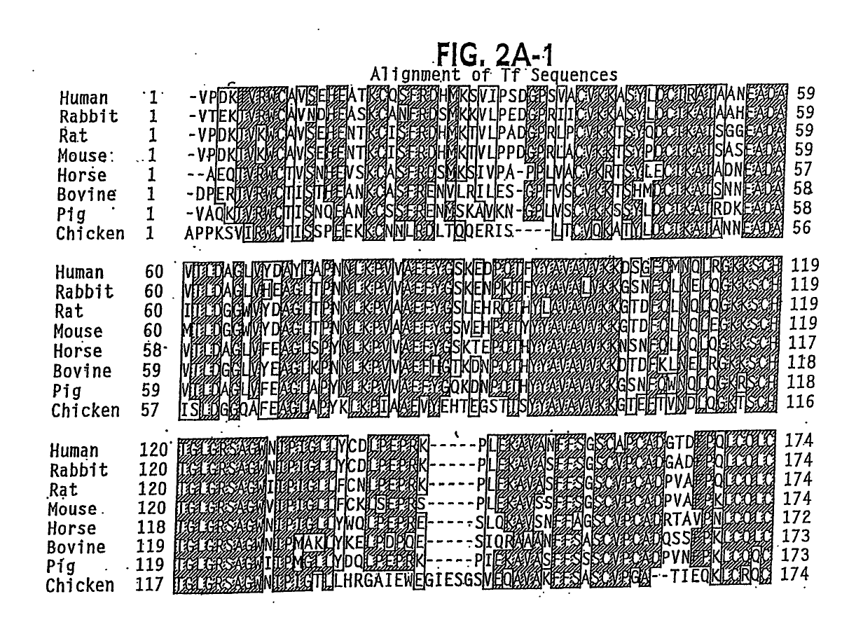

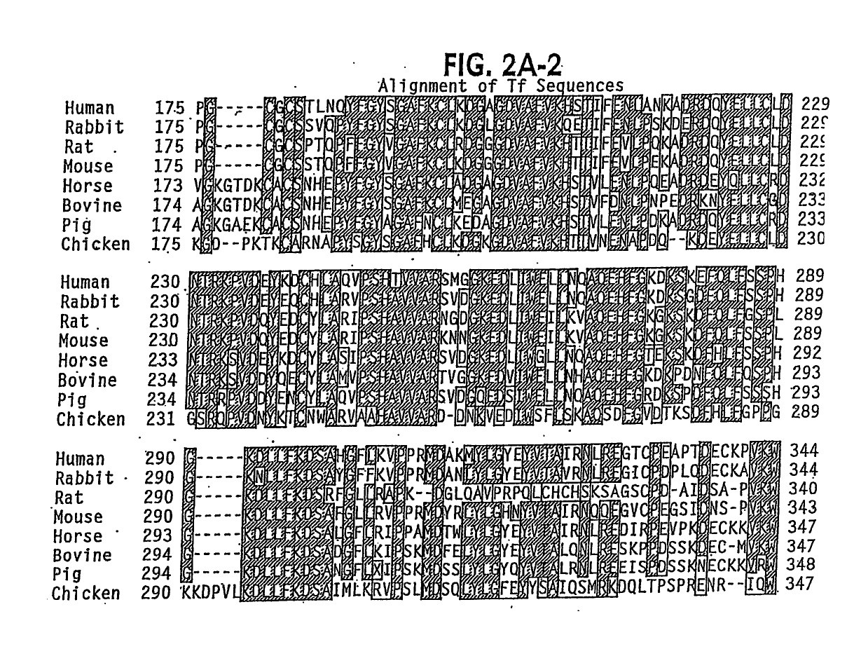

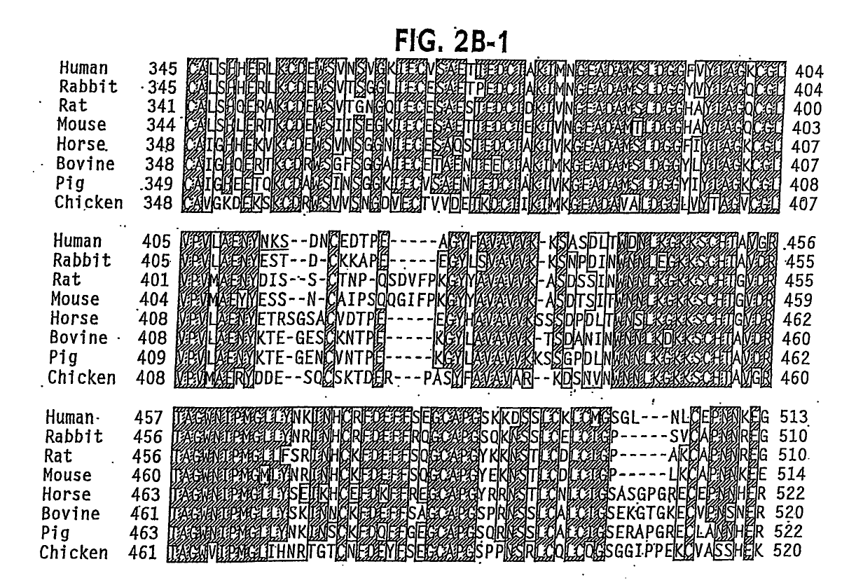

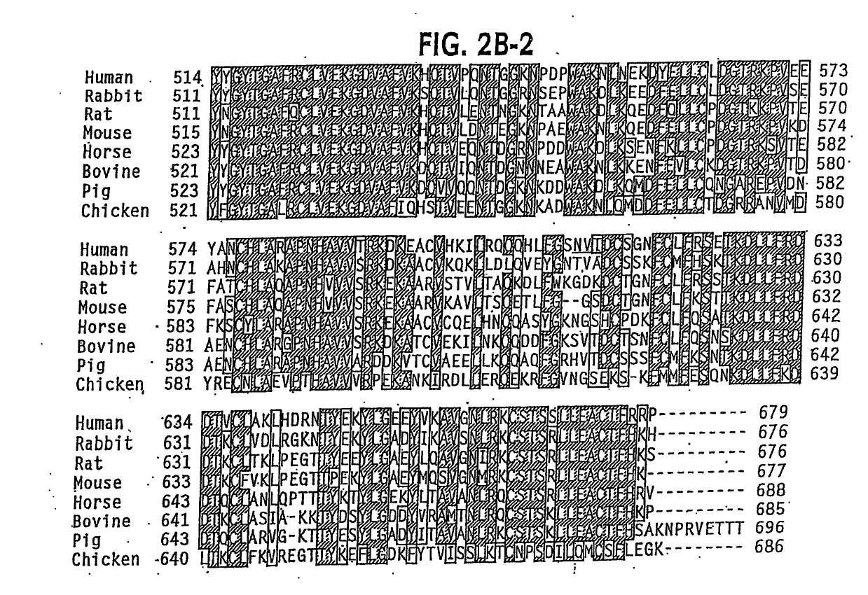

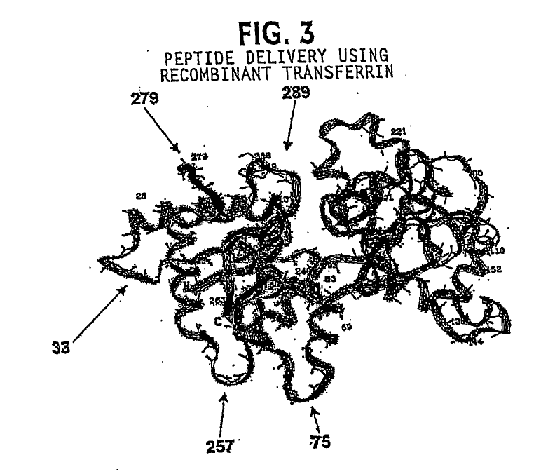

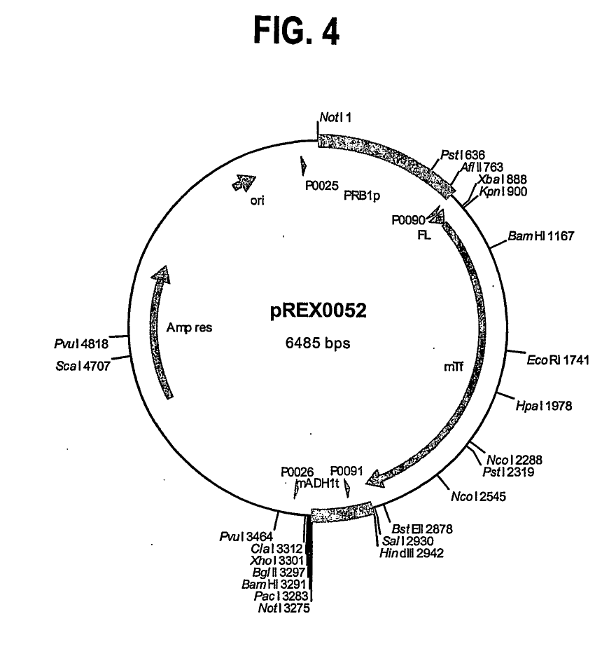

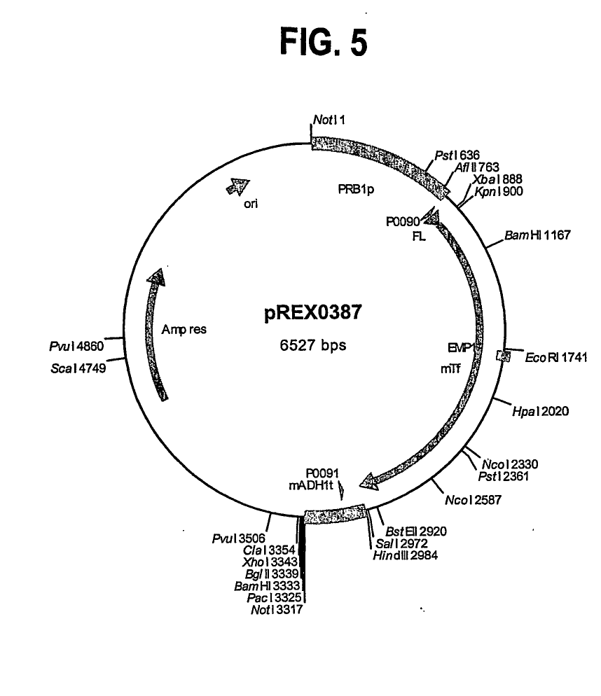

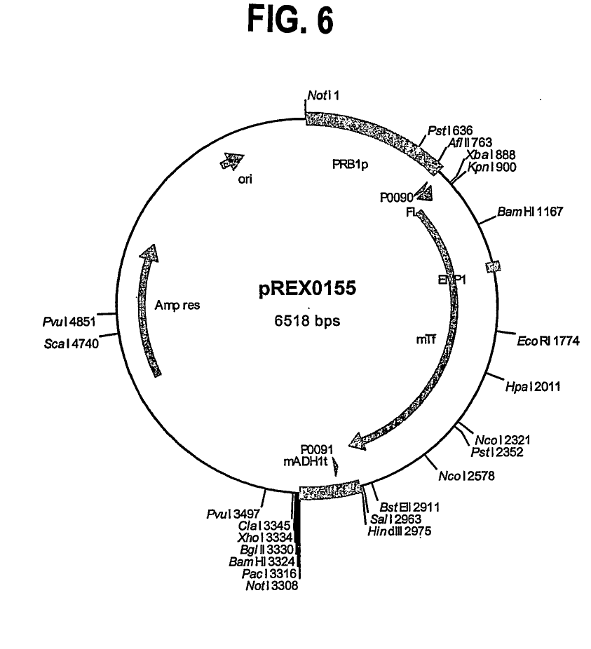

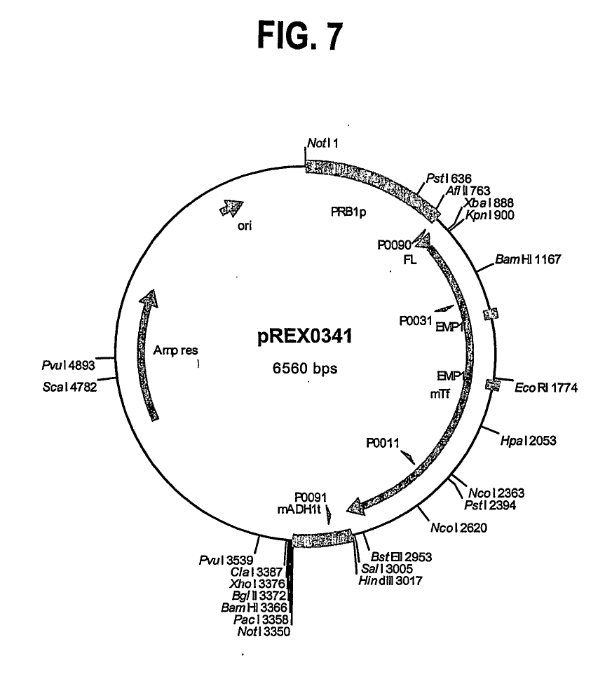

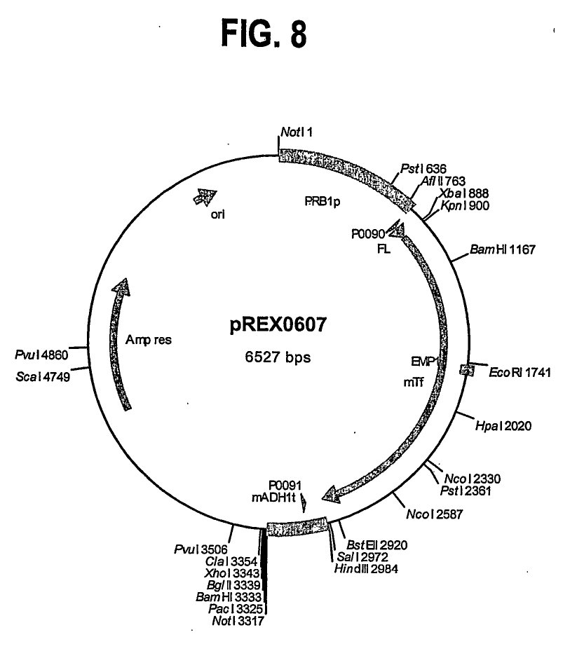

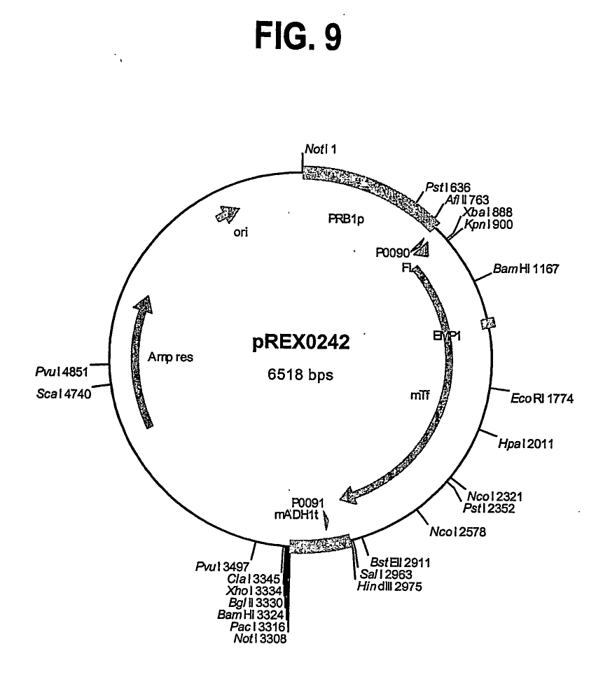

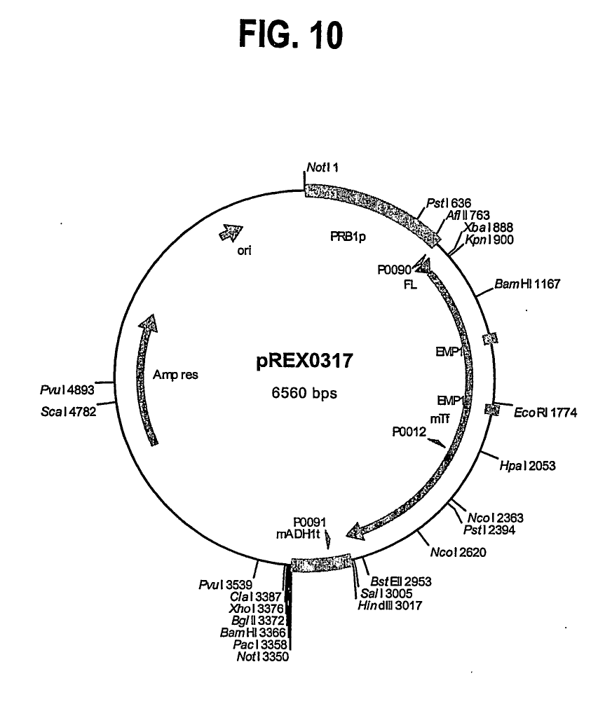

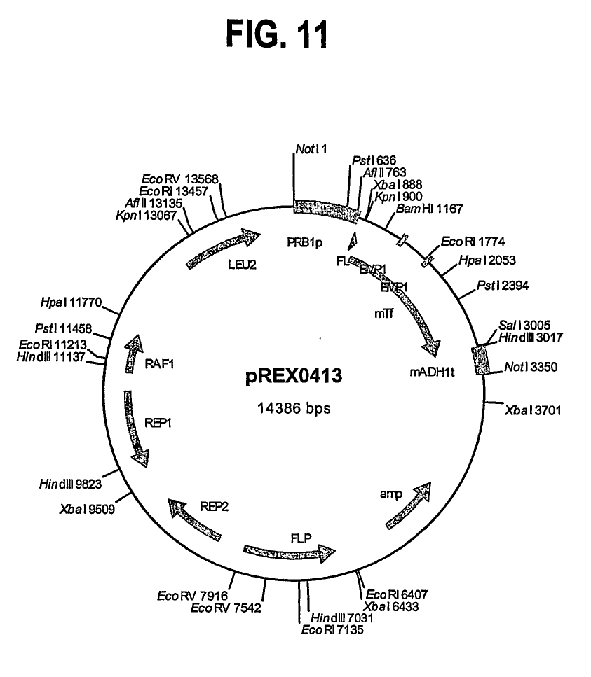

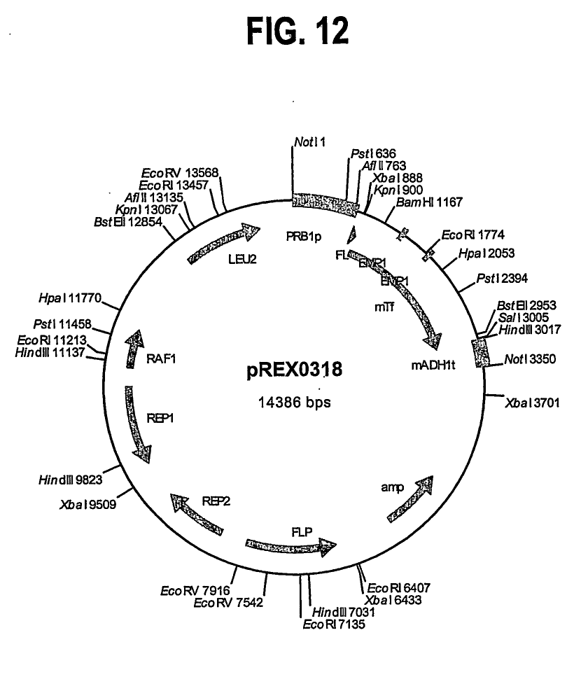

As an example, the wild-type human Tf (Tf) is a 679 amino acid protein of approximately 75 kDa (not accounting for glycosylation), with two main domains, N lobe (about 330 amino acids) and C lobe (about 340 amino acids), which appear to originate from a gene duplication. See GenBank accession numbers NM_{—}001063, XM_{—}002793, M12530, XM_{—}039845, XM_{—}039847 and S95936 (www.ncbi.nlm.nih.gov/), all of which are herein incorporated by reference in their entirety, as well as SEQ ID NOS 1, 2 and 3. The two lobes have diverged over time but retain a large degree of identity/similarity (FIG. 1).

Each of the N and C lobes is further divided into two subdomains, N1 and N2, C1 and C2. The function of Tf is to transport iron to the cells of the body. This process is mediated by the Tf receptor (“TfR”), which is expressed on all cells, particularly actively growing cells. TfR recognizes the iron bound form of Tf (two molecules of which are bound per receptor), endocytosis then occurs whereby the TfR/Tf complex is transported to the endosome, at which point the localized drop in pH results in release of bound iron and the recycling of the TfR/Tf complex to the cell surface and release of Tf (known as apoTf in its iron-unbound form). Receptor binding is through the C domain of Tf. The two glycosylation sites in the C domain do not appear to be involved in receptor binding as unglycosylated iron bound Tf does bind the receptor.

Each Tf molecule can carry two iron ions (Fe³⁺). These are complexed in the space between the N1 and N2, C1 and C2 sub domains resulting in a conformational change in the molecule. Tf crosses the blood brain barrier (BBB) via the Tf receptor.

In human transferrin, the iron binding sites comprise at least amino acids Asp 63 (Asp 82 of SEQ ID NO: 2 which includes the native Tf signal sequence), Asp 392 (Asp 411 of SEQ ID NO: 2), Tyr 95 (Tyr 114 of SEQ ID NO: 2), Tyr 426 (Tyr 445 of SEQ ID NO: 2), Tyr 188 (Tyr 207 of SEQ ID NO: 2), Tyr 514 or 517 (Tyr 533 or Tyr 536 SEQ ID NO: 2), His 249 (His 268 of SEQ ID NO: 2), and His 585 (His 604 of SEQ ID NO: 2) of SEQ ID NO: 3. The hinge regions comprise at least N lobe amino acid residues 94-96, 245-247 and/or 316-318 as well as C lobe amino acid residues 425-427, 581-582 and/or 652-658 of SEQ ID NO: 3. The carbonate binding sites comprise at least amino acids Thr 120 (Thr 139 of SEQ ID NO: 2), Thr 452 (Thr 471 of SEQ ID NO: 2), Arg 124 (Arg 143 of SEQ ID NO: 2), Arg 456 (Arg 475 of SEQ ID NO: 2), Ala 126 (Ala 145 of SEQ ID NO: 2), Ala 458 (Ala 477 of SEQ ID NO: 2), Gly 127 (Gly 146 of SEQ ID NO: 2), and Gly 459 (Gly 478 of SEQ ID NO: 2) of SEQ ID NO: 3.

A C terminal domain or lobe modified to function as an N-like domain is modified to exhibit glycosylation patterns or iron binding properties substantially like that of a native or wild-type N domain or lobe. In an illustrative embodiment, the C domain or lobe is modified so that it is not glycosylated and does not bind iron by substitution of the relevant C domain regions or amino acids to those present in the corresponding regions or sites of a native or wild-type N domain.

A Tf moiety comprising two N domains or lobes includes a Tf molecule that is modified to replace the native C domain or lobe with a native or wild-type N domain or lobe or a modified N domain or lobe or contains a C domain that has been modified to function substantially like a wild-type or modified N domain.

Analysis of the two domains by overlay of the two domains (Swiss PDB Viewer 3.7b2, Iterative Magic Fit) and by direct amino acid alignment (ClustalW multiple alignment) reveals that the two domains have diverged over time. Amino acid alignment shows 42% identity and 59% similarity between the two domains. However, approximately 80% of the N domain matches the C domain for structural equivalence. The C domain also has several extra disulfide bonds compared to the N domain.

Alignment of molecular models for the N and C domain reveals the following structural equivalents:

| | | | | | | | | | | | | | | |
|---|---|---|---|---|---|---|---|---|---|---|---|---|---|---|
| N domain | 4-24 | 36-72 | 94-136 | 138-139 | 149-164 | 168-173 | 178-198 | 219-255 | 259-260 | 263-268 | 271-275 | 279-280 | 283-288 | 309-327 |
| (1-330) | | 75-88 | | | | | 200-214 | | | | | | 290-304 | |
| C domain | 340-361 | 365-415 | 425-437 | 470-471 | 475-490 | 492-497 | 507-542 | 555-591 | 593-594 | 597-602 | 605-609 | 614-615 | 620-640 | 645-663 |
| (340-679) | | | 439-468 | | | | | | | | | | | |

The disulfide bonds for the two domains align as follows:

| N | C |
|---|---|
| | C339-C596 |
| C9-C48 | C345-C377 |
| C19-C39 | C355-C368 |
| | C402-C674 |
| | C418-C637 |
| C118-C194 | C450-C523 |
| C137-C331 | |
| | C474-C665 |
| C158-C174 | C484-C498 |
| C161-C179 | |
| C171-C177 | C495-C506 |
| C227-C241 | C563-C577 |
| | C615-C620 |

Bold aligned disulfide bonds
Italics bridging peptide

In illustrative embodiment of the invention, the second peptide or protein is transferrin. Transferrin can function as a carrier protein to extend the half life or bioavailability of the EPM peptide as well as, in some instances, delivering the EPM peptide inside a cell and/or across the blood brain barrier. In an alternate embodiment, the fusion protein includes a modified transferrin molecule, wherein the transferrin does not retain the ability to cross the blood brain barrier.

In one embodiment, the transferrin portion of the fusion protein includes at least two N terminal lobes of transferrin. In further embodiments, the transferrin portion of the fusion protein includes at least two N terminal lobes of transferrin derived from human serum transferrin.

In another embodiment, the transferrin portion of the fusion protein includes, comprises, or consists of at least two N terminal lobes of transferrin having a mutation in at least one amino acid residue selected from the group consisting of Asp63, Gly65, Tyr95, Tyr188, and His249 of SEQ ID NO: 3.

In another embodiment, the transferrin portion of the fusion protein includes a recombinant human serum transferrin N-terminal lobe mutant having a mutation at Lys206 or His207 of SEQ ID NO: 3.

In another embodiment, the transferrin portion of the fusion protein includes, comprises, or consists of at least two C terminal lobes of transferrin. In further embodiments, the transferrin portion of the fusion protein includes at least two C terminal lobes of transferrin derived from human serum transferrin.

In a further embodiment, the C terminal lobe mutant further includes a mutation of at least one of Asn413 and Asn611 of SEQ ID NO: 3, which does not allow glycosylation.

In another embodiment, the transferrin portion of the fusion protein includes at least two C terminal lobes of transferrin having a mutation in at least one amino acid residue such as, for example, Asp392, Tyr426, Tyr514, Tyr517 and His585 of SEQ ID NO: 3, wherein the mutant retains the ability to bind metal. In an alternate embodiment, the transferrin portion of the fusion protein includes at least two C terminal lobes of transferrin having a mutation in at least one amino acid residue selected from the group consisting of Tyr426, Tyr514, Tyr517 and His585 of SEQ ID NO: 3, wherein the mutant has a reduced ability to bind metal. In another embodiment, the transferrin portion of the fusion protein includes at least two C terminal lobes of transferrin having a mutation in at least one amino acid residue selected from the group consisting of Asp392, Tyr426, Tyr517 and His585 of SEQ ID NO: 3, wherein the mutant does not retain the ability to bind metal and functions substantially like an N domain.

In some embodiments, the Tf or Tf portion will be of sufficient length to increase the in vivo circulatory half-life, serum stability, in vitro solution stability, or bioavailability of the EPM peptide compared to the in vivo circulatory half-life, serum stability, in vitro solution stability or bioavailability of the EPM peptide in an unfused state. Such an increase in stability, serum half-life or bioavailability may be about a 30%, 50%, 70%, 80%, 90% or more increase over the unfused, EPM peptide. In some cases, the fusion proteins comprising modified transferrin exhibit a serum half-life of about 10-20 or more days, about 12-18 days or about 14-17 days.

When the C domain of Tf is part of the fusion protein, the two N-linked glycosylation sites, amino acid residues corresponding to N413 and N611 of SEQ ID NO: 3 may be mutated for expression in a yeast system to prevent glycosylation or hypermannosylationn and extend the serum half-life of the fusion protein and/or EPM peptide. In addition to Tf amino acids corresponding to N413 and N611, mutations may be to the adjacent residues within the N-X-S/T glycosylation site to prevent or substantially reduce glycosylation. (See, e.g., PCT US02/27637 and U.S. Pat. No. 5,986,067 to Funk et al., both of which are herein incorporated by reference in their entirety). It has also been reported that the N domain of Tf expressed in Pichia pastoris becomes O-linked glycosylated with a single hexose at S32, which also may be mutated or modified to prevent such glycosylation.

Accordingly, in one embodiment of the invention, the fusion protein includes a modified transferrin molecule wherein the transferrin exhibits reduced glycosylation, including, but not limited to, asialo- monosialo- and disialo-forms of Tf. In another embodiment, the transferrin portion of the fusion protein includes a recombinant transferrin mutant that is mutated to prevent glycosylation. In another embodiment, the transferrin portion of the fusion protein includes a recombinant transferrin mutant that is fully glycosylated. In a further embodiment, the transferrin portion of the fusion protein includes a recombinant human serum transferrin mutant that is mutated to prevent glycosylation, wherein at least one of Asn413 and Asn611 of SEQ ID NO: 3 are mutated to an amino acid which does not allow glycosylation. In another embodiment, the transferrin portion of the fusion protein includes a recombinant human serum transferrin mutant that is mutated to prevent or substantially reduce glycosylation, wherein mutations may be to the adjacent residues within the N-X-S/T glycosylation site. Moreover, glycosylation may be reduced or prevented by mutating the serine or threonine residue. Further, changing the X to proline is known to inhibit glycosylation.

As discussed below in more detail, fusion proteins of the invention may also be engineered to not bind iron and/or bind the Tf receptor. In other embodiments of the invention, the iron binding is retained and the iron binding ability of Tf may be used to deliver an EPM peptide to the inside of a cell, across an epithelial or endothelial cell membrane and/or across the BBB. These embodiments that bind iron and/or the Tf receptor will often be engineered to reduce or prevent glycosylation to extend the serum half-life of the EPM peptide. The N domain alone will not bind to TfR when loaded with iron, and the iron bound C domain will bind TfR but not with the same affinity as the whole molecule.

In another embodiment, the transferrin portion of the fusion protein includes a recombinant transferrin mutant having a mutation wherein the mutant does not retain the ability to bind metal ions. In an alternate embodiment, the transferrin portion of the fusion protein includes a recombinant transferrin mutant having a mutation wherein the mutant has a weaker binding affinity for metal ions than wild-type serum transferrin. In an alternate embodiment, the transferrin portion of the fusion protein includes a recombinant transferrin mutant having a mutation wherein the mutant has a stronger binding affinity for metal ions than wild-type serum transferrin.

In another embodiment, the transferrin portion of the fusion protein includes a recombinant transferrin mutant having a mutation wherein the mutant does not retain the ability to bind to the transferrin receptor. In an alternate embodiment, the transferrin portion of the fusion protein includes a recombinant transferrin mutant having a mutation wherein the mutant has a weaker binding affinity for the transferrin receptor than wild-type serum transferrin. In another alternate embodiment, the transferrin portion of the fusion protein includes a recombinant transferrin mutant having a mutation wherein the mutant has a stronger binding affinity for the transferrin receptor than wild-type serum transferrin.

In another embodiment, the transferrin portion of the fusion protein includes a recombinant transferrin mutant having a mutation wherein the mutant does not retain the ability to bind to carbonate ions. In an alternate embodiment, the transferrin portion of the fusion protein includes a recombinant transferrin mutant having a mutation wherein the mutant has a weaker binding affinity for carbonate ions than wild-type serum transferrin. In another alternate embodiment, the transferrin portion of the fusion protein includes a recombinant transferrin mutant having a mutation wherein the mutant has a stronger binding affinity for carbonate ions than wild-type serum transferrin.

In another embodiment, the transferrin portion of the fusion protein includes a recombinant human serum transferrin mutant having a mutation in at least one amino acid residue selected from the group consisting of Asp63, Gly65, Tyr95, Tyr188, His249, Asp392, Tyr426, Tyr514, Tyr517 and His585 of SEQ ID NO: 3, wherein the mutant retains the ability to bind metal ions. In an alternate embodiment, a recombinant human serum transferrin mutant having a mutation in at least one amino acid residue selected from the group consisting of Asp63, Gly65, Tyr95, Tyr188, His249, Asp392, Tyr426, Tyr514, Tyr517 and His585 of SEQ ID NO: 3, wherein the mutant has a reduced ability to bind metal ions. In another embodiment, a recombinant human serum transferrin mutant having a mutation in at least one amino acid residue selected from the group consisting of Asp63, Gly65, Tyr95, Tyr188, His249, Asp392, Tyr426, Tyr517 and His585 of SEQ ID NO: 3, wherein the mutant does not retain the ability to bind metal ions.

In another embodiment, the transferrin portion of the fusion protein includes a recombinant human serum transferrin mutant having a mutation at Lys206 or His207 of SEQ ID NO: 3, wherein the mutant has a stronger binding affinity for metal ions than wild-type human serum transferrin (see, e.g., U.S. Pat. No. 5,986,067, which is herein incorporated by reference in its entirety). In an alternate embodiment, the transferrin portion of the fusion protein includes a recombinant human serum transferrin mutant having a mutation at Lys206 or His207 of SEQ ID NO: 3, wherein the mutant has a weaker binding affinity for metal ions than wild-type human serum transferrin. In a further embodiment, the transferrin portion of the fusion protein includes a recombinant human serum transferrin mutant having a mutation at Lys206 or His207 of SEQ ID NO: 3, wherein the mutant does not bind metal ions.

Iron binding and/or receptor binding may be reduced or disrupted by mutation, including deletion, substitution or insertion into, amino acid residues corresponding to one or more of Tf N domain residues Asp63, Tyr95, Tyr188, His249 and/or C domain residues Asp 392, Tyr 426, Tyr 514 and/or His 585 of SEQ ID NO: 3. Iron binding may also be affected by mutation to amino acids: Lys206, His207 or Arg632 of SEQ ID NO: 3. Carbonate binding may be reduced or disrupted by mutation, including deletion, substitution or insertion into, amino acid residues corresponding to one or more of Tf N domain residues Thr120, Arg124, Ala126, Gly 127 and/or C domain residues Thr 452, Arg 456, Ala 458 and/or Gly 459 of SEQ ID NO: 3. A reduction or disruption of carbonate binding may adversely affect iron and/or receptor binding.

Binding to the Tf receptor may be reduced or disrupted by mutation, including deletion, substitution or insertion into, amino acid residues corresponding to one or more of Tf N domain residues described above for iron binding.

As discussed above, glycosylation may be reduced or prevented by mutation, including deletion, substitution or insertion into, amino acid residues corresponding to one or more of Tf C domain residues around the N-X-S/T sites corresponding to C domain residues N413 and/or N611 (See, e.g., U.S. Pat. No. 5,986,067, incorporated herein by reference). For instance, the N413 and/or N611 may be mutated to Glu residues.

In instances where the fusion proteins of the invention are not modified to prevent glycosylation, iron binding, carbonate binding and/or receptor binding, glycosylation, iron and/or carbonate ions may be stripped from or cleaved off of the fusion protein. For instance, available deglycosylases may be used to cleave glycosylation residues from the fusion protein, in particular the sugar residues attached to the Tf portion, yeast deficient in glycosylation enzymes may be used to prevent glycosylation and/or recombinant cells may be grown in the presence of an agent that prevents glycosylation, e.g., tunicamycin.

The carbohydrates on the fusion protein may also be reduced or completely removed enzymatically by treating the fusion protein with deglycosylases. Deglycosylases are well known in the art. Examples of deglycosylases include, but are not limited to, galactosidase, PNGase A, PNGase F, glucosidase, mannosidase, fucosidase, and Endo H deglycosylase.

Nevertheless, in certain circumstances, it may be preferable for oral delivery such that the Tf portion of the fusion protein be fully glycosylated

Additional mutations may be made with Tf to alter the three dimensional structure of Tf, such as modifications to the hinge region to prevent the conformational change needed for iron binding and Tf receptor recognition. For instance, mutations may be made in or around N domain amino acid residues 94-96, 245-247 and/or 316-318 as well as C domain amino acid residues 425-427, 581-582 and/or 652-658. In addition, mutations may be made in or around the flanking regions of these sites to alter Tf structure and function.

In another embodiment, the fusion protein includes a modified transferrin molecule wherein the transferrin molecule retains the ability to bind to the transferrin receptor and transport the EPM peptide inside cells. In an alternate embodiment, the fusion protein includes a modified transferrin molecule wherein the transferrin molecule does not retain the ability to bind to the transferrin receptor but maintains the ability to transport the EPM peptide inside cells.

In further embodiments, the fusion protein includes a modified transferrin molecule wherein the transferrin molecule retains the ability to bind to the transferrin receptor and transport the EPM peptide inside cells and retains the ability to cross the blood brain barrier. In an alternate embodiment, the fusion protein includes a modified transferrin molecule wherein the transferrin molecule retains the ability to cross the blood brain barrier, but does not retain the ability to bind to the transferrin receptor and transport the EPM peptide inside cells.

EPM Peptide/Albumin Fusion Protein

Any available technique may be used to produce the fusion proteins of the invention, including but not limited to, molecular techniques commonly available, for instance, those disclosed in Sambrook et al. Molecular Cloning: A Laboratory Manual, 2nd Ed., Cold Spring Harbor Laboratory Press, 1989. When carrying out nucleotide substitutions using techniques for accomplishing site-specific mutagenesis that are well known in the art, the encoded amino acid changes are preferably of a minor nature, that is, conservative amino acid substitutions, although other, non-conservative, substitutions are contemplated as well, particularly when producing a modified transferrin portion of a fusion protein, e.g., a modified Tf protein exhibiting reduced glycosylation, reduced iron binding and the like. Specifically contemplated are amino acid substitutions, small deletions or insertions, typically of one to about 30 amino acids; insertions between transferrin domains; small amino- or carboxyl-terminal extensions, such as an amino-terminal methionine residue, or small linker peptides of less than 50, 40, 30, 20 or 10 residues between transferrin domains or linking a transferrin protein and the EPM peptide or a small extension that facilitates purification, such as a poly-histidine tract, an antigenic epitope or a binding domain.

In another illustrative embodiment of the invention, the EPM peptide of the invention can be inserted or fused either directly or via a linker to albumin. In particular, the EPM peptide may constitute the N-terminal end as well as the C-terminal end of the fusion protein. In one illustrative embodiment, the EPM peptide constitutes the C-terminal part of the fusion protein. In another illustrative embodiment, the EPM peptide constitutes the N-terminal part of the fusion protein. In another illustrative embodiment, the EPM peptide portion constitutes at least one internal loop of the albumin (see, e.g., the sequence of human albumin at GenBank Accession No. AAA98797), wherein the sequence of mature albumin is residues 25-609. Identification of positions within the albumin molecule for insertion of peptides of the invention can be determined from the structure of the molecule, for instance as provided in RCBS Protein Data Bank (PDB) ID #1AO6, which is herein incorporated by reference in its entirety. Surface exposed loops within the molecule that are suitable for insertion of peptides of the invention include the following: residues 56-58, 171-173, 267-270, 311-314, 362-365, 439-442, 538-540, 561-564, wherein residue 1 is the N-terminal amino acid of mature albumin.

The albumin portion of the fusion protein includes, but is not limited to, known and yet-to-be-discovered polymorphic forms of albumin, in a particular embodiment human serum albumin (“HSA”). For example, albumin Naskapi has Lys-372 in place of Glu-372 and pro-albumin Christchurch has an altered pro-sequence. The albumin portion of the fusion protein can also include minor artificial variations in sequence such as molecules lacking one or a few residues, having conservative substitutions or minor insertions of residues, or having minor variations of amino acid structure. Thus albumin components of the fusion proteins of the invention have 80%, preferably 85%, 90%, 95% or 99%, homology with HSA are deemed to be variants. It is also preferred for such variants to be physiologically equivalent to HSA; that is to say, variants preferably share at least one pharmacological utility with HSA, for example, binding fatty acid or bilirubin or increasing the oncotic potential of the blood. The EPM peptide/albumin fusion proteins of the invention also encompass truncated forms of HSA, as described for example in U.S. Pat. No. 5,380,712 and EP 322 094 B, each of which are incorporated herein by reference. Furthermore, any putative variant, which is to be used pharmacologically should have low immunogenicity in the animal (e.g., human) being treated.

The EPM peptide/albumin fusion proteins of the invention may have N-terminal amino acids, which extend beyond (in an N-terminal direction) the portion corresponding to the N-terminal portion of HSA. For example, if the HSA portion corresponds to an N-terminal portion of mature HSA, then pre-, pro-, or pre-pro sequences may be added thereto, for example the yeast alpha-factor leader sequence. The fused leader portions described in WO 90/01063, which is incorporated herein by reference, may be used. Similarly, it is within the scope of the invention to include a linker (e.g., an amino acid linker) between the HSA portion and the EPM peptide.

In another embodiment, the amino terminal portion of the HSA molecule is so structured as to favor particularly efficient translocation and export of an EPM peptide of the invention in eukaryotic cells.

A particular embodiment of the invention encompasses a transformed host having a nucleotide sequence so arranged as to express a fusion protein as described herein. The term “so arranged,” refers to, for example, a nucleotide sequence that is downstream from an appropriate RNA polymerase binding site, is in correct reading frame with a translation start sequence and is under the control of a suitable promoter. The promoter may be homologous with or heterologous to the host. Downstream (3′) regulatory sequences may be included if desired, as is known. The host is preferably yeast (e.g., Saccharomyces spp., e.g., S. cerevisiae; Kluyveromyces spp., e.g., K. lactis; Pichia spp.; or Schizosaccharomyces spp., e.g., S. pombe) but may be any other suitable host such as E. coli, B. subtilis, Aspergillus spp., mammalian cells, plant cells, or insect cells.

In another embodiment, the amino terminal portion of the HSA molecule is so structured as to favour particularly efficient translocation and export of the EPM peptide of the invention in eukaryotic cells.

EPM Peptide/Melanotransferrin Fusion Proteins

In another illustrative embodiment, the second peptide or protein is melanotransferrin. Melanotransferrin is a glycosylated protein found at high levels in malignant melanoma cells and was originally named human melanoma antigen p97 (Brown et al., 1982, Nature, 296: 171-173). It possesses high sequence homology with human serum transferrin, human lactoferrin, and chicken transferrin (Brown et al., 1982, Nature, 296: 171-173; Rose et al., Proc. Natl. Acad. Sci. USA, 1986, 83: 1261-1265). However, unlike these receptors, no cellular receptor has been identified for melanotransferrin. Melanotransferrin reversibly binds iron and it exists in two forms, one of which is bound to cell membranes by a glycosyl phosphatidylinositol anchor while the other form is both soluble and actively secreted (Baker et al., 1992, FEBS Lett, 298: 215-218; Alemany et al., 1993, J. Cell Sci., 104: 1155-1162; Food et al., 1994, J. Biol. Chem. 274: 7011-7017). Melanotransferrin fusion proteins can be generated and utilized in a similar fashion to transferrin fusion proteins described above.

EPM Peptide/Lactoferrin Fusion Proteins

In another illustrative embodiment, the second peptide or protein is lactoferrin. Lactoferrin (Lf), a natural defense iron-binding protein, has been found to possess antibacterial, antimycotic, antiviral, antineoplastic and anti-inflammatory activity. The protein is present in exocrine secretions that are commonly exposed to normal flora: milk, tears, nasal exudate, saliva, bronchial mucus, gastrointestinal fluids, cervico-vaginal mucus and seminal fluid. Additionally, Lf is a major constituent of the secondary specific granules of circulating polymorphonuclear neutrophils (PMNs). The apoprotein is released on degranulation of the PMNs in septic areas. A principal function of Lf is that of scavenging free iron in fluids and inflamed areas so as to suppress free radical-mediated damage and decrease the availability of the metal to invading microbial and neoplastic cells. In a study that examined the turnover rate of ¹²⁵I Lf in adults, it was shown that Lf is rapidly taken up by the liver and spleen, and the radioactivity persisted for several weeks in the liver and spleen (Bennett et al. (1979), Clin. Sci. (Lond.) 57: 453-460). Lactoferrin fusion proteins can be generated and utilized in a similar fashion to transferring described above.

Additional Illustrative EPM Peptide/Second Peptide or Protein Fusion Proteins

It will be readily understood to those of ordinary skill in the art that an EPM peptide can be fused to or inserted in any desired second peptide or protein including, but not limited to, maltose binding protein NP), green fluorescent protein (GFP), and glutathione S-transferase (GST) using the techniques described herein. Therefore, the second peptide or protein is not limited to embodiments specified herein. Thus, in another illustrative embodiment, the fusion protein is a GST-EPM peptide fusion protein in which the EPM peptide sequence is fused to the C-terminus of the GST sequence.

In yet another illustrative embodiment, the fusion protein is a modified immunoglobulin-EPM peptide fusion protein in which the EPM peptide sequences are fused to sequences derived from a member of the immunoglobulin protein family. The immunoglobulin-EPM peptide fusion proteins of the invention can be incorporated into pharmaceutical compositions and administered to a subject to inhibit or suppress EPM peptide-mediated signal transduction in vivo. The immunoglobulin-EPM peptide fusion proteins can be used therapeutically for both the treatment of proliferative and differentiative disorders, as well as modulating (e.g., promoting or inhibiting) cell survival. Moreover, the immunoglobulin-EPM peptide fusion proteins of the invention can be used as immunogens to produce anti-EPM peptide antibodies in a subject.

Fusion Proteins Comprising One or More EPM Peptides and One or More Additional Therapeutics

The invention also encompasses a fusion protein wherein one or more EPM peptides is inserted in or fused to a second peptide or protein, and additionally, the fusion protein comprises one or more additional therapeutics (e.g., neuropharmaceutical agent) fused to or inserted in a second peptide or protein. In another embodiment, the fusion protein includes an EPM peptide at the carboxyl terminus of a second peptide or protein linked to an additional therapeutic (e.g., a neuropharmaceutical agent) at the amino terminus of a second peptide or protein. In an alternate embodiment, the fusion protein includes an EPM peptide at the amino terminus linked of a second peptide or protein and a neuropharmaceutical agent at the carboxy terminus of a second peptide or protein. In specific embodiments, the neuropharmaceutical agent is either a nerve growth factor or ciliary neurotrophic factor.

In a further embodiment, the fusion proteins can contain an additional therapeutic that is a peptide, peptide fragment, or peptide variant of proteins or antibodies, wherein the variant or fragment retains at least one biological or therapeutic activity. The fusion proteins can also contain an EPM peptide that can be the peptide fragments or peptide variants at least about 4, at least 5, at least 6, at least 7, at least 8, at least 9, at least 10, at least 11, at least 12, at least 13, at least 14, at least 15 amino acids in length fused to the N and/or C termini, inserted within, or inserted into a loop of a second peptide or protein.

The fusion proteins of the invention may contain one or more additional therapeutic peptides. In a particular embodiment, increasing the number of peptides can enhance the function of the peptides fused to second peptide or protein and the function of the entire fusion protein. The peptides may be used to make a bi- or multi-functional fusion protein by including peptide or protein domains with multiple functions. For instance, a multi-functional fusion protein can be made with an EPM peptide and a second protein to target the fusion protein to a specific target. Other peptides may be used to induce the immune response of a cellular system, or induce an antiviral, antibacterial, or anti-pathogenic response. In a particular embodiment, at least two EPM peptide portions are fused to a second peptide or protein to activate a receptor and induce an immune response.

The EPM peptide corresponding to an EPM peptide portion of a fusion protein of the invention can be modified by the attachment of one or more oligosaccharide groups. The modification referred to as glycosylation can significantly affect the physical properties of proteins and can be important in protein stability, secretion, and localization. Glycosylation occurs at specific locations along the polypeptide backbone. There are usually two major types of glycosylation: glycosylation characterized by O-linked oligosaccharides, which are attached to serine or threonine residues; and glycosylation characterized by N-linked oligosaccharides, which are attached to asparagine residues in an Asn-X-Ser/Thr sequence, where X can be any amino acid except proline. Variables such as protein structure and cell type influence the number and nature of the carbohydrate units within the chains at different glycosylation sites. Glycosylation isomers are also common at the same site within a given cell type. For example, several types of human interferon are glycosylated.

Proteins in addition to an EPM peptide corresponding to an additional therapeutic protein portion of a fusion protein of the invention, as well as analogs and variants thereof, may be modified so that glycosylation at one or more sites is altered as a result of manipulation(s) of their nucleic acid sequence by the host cell in which they are expressed, or due to other conditions of their expression. For example, glycosylation isomers may be produced by abolishing or introducing glycosylation sites (e.g., by substitution or deletion of amino acid residues) such as substitution of glutamine for asparagine, or unglycosylated recombinant proteins may be produced by expressing the proteins in host cells that will not glycosylate them, e.g. in glycosylation-deficient yeast. These approaches are known in the art.

In other embodiments, the fusion proteins of the invention are capable of a therapeutic activity and/or biologic activity, corresponding to the therapeutic activity and/or biologic activity of the EPM peptide described elsewhere in this application. In further embodiments, the therapeutically active protein portions of the fusion proteins of the invention are fragments or variants of additional therapeutic sequences.

In one embodiment, additional therapeutic are biologically active components of the fusion protein. Additional Therapeutics exhibit complementary activity or synergistic activity, but not necessarily identical, to an activity of an EPM peptide used in the invention. The biological activity of the additional therapeutics may include an improved desired activity, or a decreased undesirable activity.

Nucleic Acids

The invention also provides nucleic acid molecules encoding fusion proteins comprising a second peptide or protein or a portion of a second peptide or protein covalently linked or joined to an EPM peptide or a fragment thereof.

Another embodiment of the invention encompasses an isolated nucleic acid molecule that encodes the EPM peptide of the invention, or biologically active portions thereof, as well as nucleic acid fragments sufficient for use as hybridization probes to identify EPM peptide-encoding nucleic acids (e.g., EPM peptide mRNA) and fragments for use as PCR primers for the amplification or mutation of EPM peptide nucleic acid molecules. The nucleic acid molecule can be single-stranded or double-stranded, but preferably is double-stranded DNA. One embodiment of the invention encompasses one or more nucleic acid molecules that differ from the EMP-1 nucleotide sequence shown in SEQ ID NO:4 due to degeneracy of the genetic code to generate an EPM peptide that retains the activity or has greater activity than EMP-1.

Another aspect of the invention pertains to nucleic acid molecules encoding EPM peptides that contain changes in amino acid residues that are not essential for activity. Such EPM peptides differ in amino acid sequence from the native EMP-1 yet retain biological activity. In one embodiment, the isolated nucleic acid molecule comprises a nucleotide sequence encoding a peptide, wherein the peptide comprises an amino acid sequence at least about 45% homologous to the EMP-1 amino acid sequence. Preferably, the protein encoded by the nucleic acid molecule is at least about 60% homologous to the EMP-1 amino acid sequence, more preferably at least about 70% homologous, at least about 80% homologous, at least about 90% homologous, and preferably at least about 95% homologous to that given EMP-1 peptide.

An isolated nucleic acid molecule encoding an EPM peptide homologous to a given EMP-1 peptide can be created by introducing one or more nucleotide substitutions, additions or deletions into the corresponding EMP-1 nucleotide sequence, such that one or more amino acid substitutions, additions or deletions are introduced into the encoded protein.

In another embodiment, an isolated nucleic acid molecule of the invention is at least 9 nucleotides in length and hybridizes under stringent conditions to the nucleic acid molecule comprising at least one EPM peptide nucleotide sequence. In another embodiment, an isolated nucleic acid molecule of the invention hybridizes to the coding region.

Host cells and vectors for replicating the nucleic acid molecules and for expressing the encoded fusion proteins are also provided. Any vectors or host cells may be used, whether prokaryotic or eukaryotic, but eukaryotic expression systems, in particular yeast expression systems, may be preferred. Many vectors and host cells are known in the art for such purposes. It is well within the skill of the art to select an appropriate set for the desired application.

Techniques for isolating DNA sequences encoding EPM peptides using probe-based methods are conventional techniques and are well known to those skilled in the art. Probes for isolating such DNA sequences may be based on published DNA or protein sequences (see, e.g., Baldwin, G. S. (1993) Comparison of Transferrin Sequences from Different Species. Comp. Biochem. Physiol. 106B/1:203-218 and all references cited therein, which are hereby incorporated by reference in their entirety). Alternatively, the polymerase chain reaction (PCR) method disclosed by Mullis et al. (U.S. Pat. No. 4,683,195) and Mullis (U.S. Pat. No. 4,683,202), incorporated herein by reference, may be used. The choice of library and selection of probes for the isolation of such DNA sequences is within the level of ordinary skill in the art.

As known in the art similarity between two polynucleotides or polypeptides is determined by comparing the nucleotide or amino acid sequence and its conserved nucleotide or amino acid substitutes of one polynucleotide or polypeptide to the sequence of a second polynucleotide or polypeptide. Also known in the art is “identity” which means the degree of sequence relatedness between two polypeptide or two polynucleotide sequences as determined by the identity of the match between two strings of such sequences. Both identity and similarity can be readily calculated (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

While there exist a number of methods to measure identity and similarity between two polynucleotide or polypeptide sequences, the terms “identity” and “similarity” are well known to skilled artisans (Sequence Analysis in Molecular Biology, von Heinje, G., Academic Press, 1987; Sequence Analysis Primer, Gribskov, M. and Devereux, J., eds., M Stockton Press, New York, 1991; and Carillo, H., and Lipman, D., SIAM J. Applied Math., 48: 1073 (1988). Methods commonly employed to determine identity or similarity between two sequences include, but are not limited to those disclosed in Guide to Huge Computers, Martin J. Bishop, ed., Academic Press, San Diego, 1994, and Carillo, H., and Lipman, D., SIAM J. Applied Math. 48:1073 (1988).

Preferred methods to determine identity are designed to give the largest match between the two sequences tested. Methods to determine identity and similarity are codified in computer programs. Preferred computer program methods to determine identity and similarity between two sequences include, but are not limited to, GCG program package (Devereux, et al., Nucl. Acid Res. 12(1):387 (1984)), BLASTP, BLASTN, FASTA (Atschul, et al., J. Mol. Biol. 215:403 (1990)). The degree of similarity or identity referred to above is determined as the degree of identity between the two sequences, often indicating a derivation of the first sequence from the second. The degree of identity between two nucleic acid sequences may be determined by means of computer programs known in the art such as GAP provided in the GCG program package (Needleman and Wunsch J. Mol. Biol. 48:443-453 (1970)). For purposes of determining the degree of identity between two nucleic acid sequences for the present invention, GAP is used with the following settings: GAP creation penalty of 5.0 and GAP extension penalty of 0.3.

The invention further encompasses methods for producing a fusion protein of the invention using nucleic acid molecules. In general terms, the production of a recombinant form of a protein typically involves the following steps.

A nucleic acid molecule is first obtained that encodes a fusion protein of the invention. The nucleic acid molecule is then preferably placed in operable linkage with suitable control sequences, as described above, to form an expression unit containing the protein open reading frame. The expression unit is used to transform a suitable host and the transformed host is cultured under conditions that allow the production of the recombinant protein. Optionally the recombinant protein is isolated from the medium or from the cells; recovery and purification of the protein may not be necessary in some instances where some impurities may be tolerated.

Each of the foregoing steps can be accomplished in a variety of ways. For example, the construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier and are otherwise known to persons skilled in the art. Suitable restriction sites can, if not normally available, be added to the ends of the coding sequence so as to provide an excisable gene to insert into these vectors. A skilled artisan can readily adapt any host/expression system known in the art for use with the nucleic acid molecules of the invention to produce a desired recombinant protein.

Any expression system may be used, including yeast, bacterial, animal, plant, eukaryotic and prokaryotic systems. In some embodiments, yeast, mammalian cell culture and transgenic animal or plant production systems are preferred. In other embodiments, yeast systems that have been modified to reduce native yeast glycosylation, hyper-glycosylation or proteolytic activity may be used.

Codon Optimization

The degeneracy of the genetic code permits variations of the nucleotide sequence of an EMP-1 protein while still producing a polypeptide having the identical amino acid sequence as the polypeptide encoded by the native DNA sequence. The procedure, known as “codon optimization” (described in U.S. Pat. No. 5,547,871, which is incorporated herein by reference in its entirety) provides one with a means of designing such an altered DNA sequence. The design of codon optimized genes should take into account a variety of factors, including the frequency of codon usage in an organism, nearest neighbor frequencies, RNA stability, the potential for secondary structure formation, the route of synthesis and the intended future DNA manipulations of that gene. In particular, available methods may be used to alter the codons encoding a given fusion protein with those most readily recognized by yeast when yeast expression systems are used.

The degeneracy of the genetic code permits the same amino acid sequence to be encoded and translated in many different ways. For example, leucine, serine and arginine are each encoded by six different codons, while valine, proline, threonine, alanine and glycine are each encoded by four different codons. However, the frequency of use of such synonymous codons varies from genome to genome among eukaryotes and prokaryotes. For example, synonymous codon-choice patterns among mammals are very similar, while evolutionarily distant organisms such as yeast (such as S. cerevisiae), bacteria (such as E. coli) and insects (such as D. melanogaster) reveal a clearly different pattern of genomic codon use frequencies (Grantham, R., et al., Nucl. Acid Res., 8, 49-62 (1980); Grantham, R., et al, Nucl. Acid Res., 9, 43-74 (1981); Maroyama, T., et al., Nucl. Acid Res., 14, 151-197 (1986); Aota, S., et al., Nucl. Acid Res., 16, 315-402 (1988); Wada, K., et al., Nucl. Acid Res., 19 Supp., 1981-1985 (1991); Kurland, C. G., FEBS Lett., 285, 165-169 (1991)). These differences in codon-choice patterns appear to contribute to the overall expression levels of individual genes by modulating peptide elongation rates. (Kurland, C. G., FEBS Lett., 285, 165-169 (1991); Pedersen, S., EMBO J., 3, 2895-2898 (1984); Sorensen, M. A., J. Mol. Biol., 207, 365-377 (1989); Randall, L. L., et al., Eur. J. Biochem., 107, 375-379 (1980); Curran, J. F., and Yarus, M., J. Mol. Biol., 209, 65-77 (1989); Varenne, S., et al., J. Mol. Biol., 180, 549-576 (1984), Varenne, S., et al., J. Mol, Biol., 180, 549-576 (1984); Garel, J.-P., J. Theor. Biol., 43, 211-225 (1974); Ikemura, T., J. Mol. Biol., 146, 1-21 (1981); Ikemura, T., J. Mol. Biol., 151, 389-409 (1981)).

The preferred codon usage frequencies for a synthetic gene should reflect the codon usages of nuclear genes derived from the exact (or as closely related as possible) genome of the cell/organism that is intended to be used for recombinant protein expression, particularly that of yeast species. As discussed above, in one preferred embodiment the human second peptide or protein sequence is codon optimized, before or after modification as herein described for yeast expression as may be the EPM peptide nucleotide sequence(s).

Vectors

Expression units for use in the present invention will generally comprise the following elements, operably linked in a 5′ to 3′ orientation: a transcriptional promoter, a secretory signal sequence, a DNA sequence encoding a fusion protein comprising a second peptide or protein or a portion of a second peptide or protein joined to a DNA sequence encoding an EPM peptide and a transcriptional terminator. As discussed above, any arrangement of the EPM peptide fused to or within an EMP-1 portion may be used in the vectors of the invention. The selection of suitable promoters, signal sequences and terminators will be determined by the selected host cell and will be evident to one skilled in the art and are discussed more specifically below.

Suitable yeast vectors for use in the present invention are described in U.S. Pat. No. 6,291,212 and include YRp7 (Struhl et al., Proc. Natl. Acad. Sci. USA 76: 1035-1039, 1978), YEp13 (Broach et al., Gene 8: 121-133, 1979), pJDB249 and pJDB219 (Beggs, Nature 275:104-108, 1978), pPPC0005, pSeCHSA, pScNHSA, pC4 and derivatives thereof. Useful yeast plasmid vectors also include pRS403-406, pRS413-416 and the Pichia vectors available from Stratagene Cloning Systems, La Jolla, Calif. 92037, USA. Plasmids pRS403, pRS404, pRS405 and pRS406 are Yeast Integrating plasmids (YIps) and incorporate the yeast selectable markers HIS3, TRP1, LEU2 and URA3. Plasmids pRS413˜41.6 are Yeast Centromere plasmids (YCps).

Such vectors will generally include a selectable marker, which may be one of any number of genes that exhibit a dominant phenotype for which a phenotypic assay exists to enable transformants to be selected. Preferred selectable markers are those that complement host cell auxotrophy, provide antibiotic resistance or enable a cell to utilize specific carbon sources, and include LEU2 (Broach et al. ibid.), URA3 (Botstein et al., Gene 8: 17, 1979), HIS3 (Struhl et al, ibid.) or POT1 (Kawasaki and Bell, EP 171,142). Other suitable selectable markers include the CAT gene, which confers chloramphenicol resistance on yeast cells. Preferred promoters for use in yeast include promoters from yeast glycolytic genes (Hitzeman et al., J. Biol. Chem. 225: 12073-12080, 1980; Alber and Kawasaki, J. Mol. Appl. Genet. 1: 419-434, 1982; Kawasaki, U.S. Pat. No. 4,599,311) or alcohol dehydrogenase genes (Young et al., in Genetic Engineering of Microorganisms for Chemicals, Hollaender et al., (eds.), p. 355, Plenum, N.Y., 1982; Ammerer, Meth. Enzymol. 101: 192-201, 1983). In this regard, particularly preferred promoters are the TP11 promoter (Kawasaki, U.S. Pat. No. 4,599,311) and the ADH2-4^{C} (see U.S. Pat. No. 6,291,212 promoter (Russell et al., Nature 304: 652-654, 1983). The expression units may also include a transcriptional terminator. A preferred transcriptional terminator is the TP11 terminator (Alber and Kawasaki, ibid.). Other preferred vectors and preferred components such as promoters and terminators of a yeast expression system are disclosed in European Patents EP 0258067, EP 0286424, EP0317254, EP 0387319, EP 0386222, EP 0424117, EP 0431880, and EP 1002095; European Patent Publications EP 0828759, EP 0764209, EP 0749478, and EP 0889949; PCT Publication WO 00/44772 and WO 94/04687; and U.S. Pat. Nos. 5,739,007; 5,637,504; 5,302,697; 5,260,202; 5,667,986; 5,728,553; 5,783,423; 5,965,386; 6150,133; 6,379,924; and 5,714,377; each of which are herein incorporated by reference in their entirety.

In addition to yeast, fusion proteins of the present invention can be expressed in filamentous fungi, for example, strains of the fungi Aspergillus. Examples of useful promoters include those derived from Aspergillus nidulans glycolytic genes, such as the adh3 promoter (McKnight et al., EMBO J. 4: 2093-2099, 1985) and the tpia promoter. An example of a suitable terminator is the adh3 terminator (McKnight et al., ibid.). The expression units utilizing such components may be cloned into vectors that are capable of insertion into the chromosomal DNA of Aspergillus, for example.

Mammalian expression vectors for use in carrying out the present invention will include a promoter capable of directing the transcription of the fusion protein. Preferred promoters include viral promoters and cellular promoters. Preferred viral promoters include the major late promoter from adenovirus 2 (Kaufman and Sharp, Mol. Cell. Biol. 2: 1304-13199, 1982) and the SV40 promoter (Subramani et al., Mol. Cell. Biol. 1: 854-864, 1981). Preferred cellular promoters include the mouse metallothionein 1 promoter (Palmiter et al., Science 222: 809-814, 1983) and a mouse Vκ (see U.S. Pat. No. 6,291,212) promoter (Grant et al., Nuc. Acids Res. 15: 5496, 1987). A particularly preferred promoter is a mouse V_{H} (see U.S. Pat. No. 6,291,212) promoter (Loh et al., ibid.). Such expression vectors may also contain a set of RNA splice sites located downstream from the promoter and upstream from the DNA sequence encoding the transferrin fusion protein. Preferred RNA splice sites may be obtained from adenovirus and/or immunoglobulin genes.

Also contained in the expression vectors is a polyadenylation signal located downstream of the coding sequence of interest. Polyadenylation signals include the early or late polyadenylation signals from SV40 (Kaufman and Sharp, ibid.), the polyadenylation signal from the adenovirus 5 E1B region and the human growth hormone gene terminator (DeNoto et al., Nucl. Acid Res. 9: 3719-3730, 1981). A particularly preferred polyadenylation signal is the V_{H} (see U.S. Pat. No. 6,291,212) gene terminator (Loh et al., ibid.). The expression vectors may include a noncoding viral leader sequence, such as the adenovirus 2 tripartite leader, located between the promoter and the RNA splice sites. Preferred vectors may also include enhancer sequences, such as the SV40 enhancer and the mouse μ (see U.S. Pat. No. 6,291,212) enhancer (Gillies, Cell 33: 717-728, 1983). Expression vectors may also include sequences encoding the adenovirus VA RNAs.

Transformation

Techniques for transforming fungi are well known in the literature, and have been described, for instance, by Beggs (ibid.), Hinnen et al. (Proc. Natl. Acad. Sci. USA 75: 1929-1933, 1978), Yelton et al., (Proc. Natl. Acad. Sci. USA 81: 1740-1747, 1984), and Russell (Nature 301: 167-169, 1983). Other techniques for introducing cloned DNA sequences into fungal cells, such as electroporation (Becker and Guarente, Methods in Enzymol. 194: 182-187, 1991) may be used. The genotype of the host cell will generally contain a genetic defect that is complemented by the selectable marker present on the expression vector. Choice of a particular host and selectable marker is well within the level of ordinary skill in the art.

Cloned DNA sequences comprising modified Tf fusion proteins of the invention may be introduced into cultured mammalian cells by, for example, calcium phosphate-mediated transfection (Wigler et al., Cell 14: 725, 1978; Corsaro and Pearson, Somatic Cell Genetics 7: 603, 1981; Graham and Van der Eb, Virology 52: 456, 1973.) Other techniques for introducing cloned DNA sequences into mammalian cells, such as electroporation (Neumann et al., EMBO J. 1: 841-845, 1982), or lipofection may also be used. In order to identify cells that have integrated the cloned DNA, a selectable marker is generally introduced into the cells along with the gene or cDNA of interest. Preferred selectable markers for use in cultured mammalian cells include genes that confer resistance to drugs, such as neomycin, hygromycin, and methotrexate. The selectable marker may be an amplifiable selectable marker. A preferred amplifiable selectable marker is the DHFR gene. A particularly preferred amplifiable marker is the DBFR^{r} (see U.S. Pat. No. 6,291,212) cDNA (Simonsen and Levinson, Proc. Natl. Acad. Sci. USA 80: 2495-2499, 1983). Selectable markers are reviewed by Thilly (Mammalian Cell Technology, Butterworth Publishers, Stoneham, Mass.) and the choice of selectable markers is well within the level of ordinary skill in the art.

Host Cells

The invention also includes a cell, preferably a yeast cell transformed to express a fusion protein of the invention. In addition to the transformed host cells themselves, the present invention also includes a culture of those cells, preferably a monoclonal (clonally homogeneous) culture, or a culture derived from a monoclonal culture, in a nutrient medium. If the polypeptide is secreted, the medium will contain the polypeptide, with the cells, or without the cells if they have been filtered or centrifuged away.

Host cells for use in practicing the invention include eukaryotic cells, and in some cases prokaryotic cells, capable of being transformed or transfected with exogenous DNA and grown in culture, such as cultured mammalian, insect, fungal, plant and bacterial cells.

Fungal cells, including species of yeast (e.g., Saccharomyces spp., Schizosaccharomyces spp., Pichia spp.) may be used as host cells within the present invention. Examples of fingi including yeasts contemplated to be useful in the practice of the invention as hosts for expressing the fusion protein of the inventions are Pichia (some species of which were formerly classified as Hansenula), Saccharomyces, Kluyveromyces, Aspergillus, Candida, Torulopsis, Torulaspora, Schizosaccharomyces, Citeroinyces, Pachysolen, Zygosaccharomyces, Debaromyces, Trichoderina, Cephalosporium, Humicola, Mucor, Neurospora, Yarrowia, Metschunikowia, Rhodosporidium, Leucosporidium, Botryoascus, Sporidiobolus, Endomycopsis, and the like. Examples of Saccharomyces spp. are S. cerevisiae, S. italicus and S. rouxii. Examples of KIuyveromyces spp. are K. fragilis, K. lactis and K. marxianus. A suitable Torulaspora species is T. delbrueckii. Examples of Pichia spp. are P. angusta (formerly H. polymorpha), P. anomala (formerly H. anomala) and P. pastoris.

Particularly useful host cells to produce the fusion proteins of the invention are the methylotrophic Pichia pastoris (Steinlein et al. (1995) Protein Express. Purif 6:619-624). Pichia pastoris has been developed to be an outstanding host for the production of foreign proteins since its alcohol oxidase promoter was isolated and cloned; its transformation was first reported in 1985. P. pastoris can utilize methanol as a carbon source in the absence of glucose. The P. pastoris expression system can use the methanol-induced alcohol oxidase (AOX1) promoter, which controls the gene that codes for the expression of alcohol oxidase, the enzyme which catalyzes the first step in the metabolism of methanol. This promoter has been characterized and incorporated into a series of P. pastoris expression vectors. Since the proteins produced in P. pastoris are typically folded correctly and secreted into the medium, the fermentation of genetically engineered P. pastoris provides an excellent alternative to E. coli expression systems. A number of proteins have been produced using this system, including tetanus toxin fragment, Bordatella pertussis pertactin, human serum albumin and lysozyme.

Strains of the yeast Saccharomyces cerevisiae are another preferred host. In a preferred embodiment, a yeast cell, or more specifically, a Saccharomyces cerevisiae host cell that contains a genetic deficiency in a gene required for asparagine-linked glycosylation of glycoproteins is used. S. cerevisiae host cells having such defects may be prepared using standard techniques of mutation and selection, although many available yeast strains have been modified to prevent or reduce glycosylation or hypermannosylation. Ballou et al. (J. Biol. Chem. 255: 5986-5991, 1980) have described the isolation of mannoprotein biosynthesis mutants that are defective in genes which affect asparagine-linked glycosylation. Gentzsch and Tanner (Glycobiology 7:481-486, 1997) have described a family of at least six genes (PMT1-6) encoding enzymes responsible for the first step in O-glycosylation of proteins in yeast. Mutants defective in one or more of these genes show reduced O-linked glycosylation and/or altered specificity of O-glycosylation.

To optimize production of the heterologous proteins, it is also preferred that the host strain carries a mutation, such as the S. cerevisiae pep4 mutation (Jones, Genetics 85: 23-33, 1977), which results in reduced proteolytic activity. Host strains containing mutations in other protease encoding regions are particularly useful to produce large quantities of the fusion proteins of the invention.

Host cells containing DNA constructs of the present invention are grown in an appropriate growth medium. As used herein, the term “appropriate growth medium” means a medium containing nutrients required for the growth of cells. Nutrients required for cell growth may include a carbon source, a nitrogen source, essential amino acids, vitamins, minerals and growth factors. The growth medium will generally select for cells containing the DNA construct by, for example, drug selection or deficiency in an essential nutrient which is complemented by the selectable marker on the DNA construct or co-transfected with the DNA construct. Yeast cells, for example, are preferably grown in a chemically defined medium, comprising a carbon source, e.g. sucrose, a non-amino acid nitrogen source, inorganic salts, vitamins and essential amino acid supplements. The pH of the medium is preferably maintained at a pH greater than 2 and less than 8, preferably at pH 5.5-6.5. Methods for maintaining a stable pH include buffering and constant pH control. Preferred buffering agents include succinic acid and Bis-Tris (Sigma Chemical Co., St. Louis, Mo.). Yeast cells having a defect in a gene required for asparagine-linked glycosylation are preferably grown in a medium containing an osmotic stabilizer. A preferred osmotic stabilizer is sorbitol supplemented into the medium at a concentration between 0.1 M and 1.5 M, preferably at 0.5 M or 1.0 M.

Cultured mammalian cells are generally grown in commercially available serum-containing or serum-free media. Selection of a medium appropriate for the particular cell line used is within the level of ordinary skill in the art. Transfected mammalian cells are allowed to grow for a period of time, typically 1-2 days, to begin expressing the DNA sequence(s) of interest. Drug selection is then applied to select for growth of cells that are expressing the selectable marker in a stable fashion. For cells that have been transfected with an amplifiable selectable marker the drug concentration may be increased in a stepwise manner to select for increased copy number of the cloned sequences, thereby increasing expression levels.

Baculovirus/insect cell expression systems may also be used to produce the modified Tf fusion proteins of the invention. The BacPAK™ Baculovirus Expression System (BD Biosciences (Clontech)) expresses recombinant proteins at high levels in insect host cells. The target gene is inserted into a transfer vector, which is cotransfected into insect host cells with the linearized BacPAK6 viral DNA. The BacPAK6 DNA is missing an essential portion of the baculovirus genome. When the DNA recombines with the vector, the essential element is restored and the target gene is transferred to the baculovirus genome. Following recombination, a few viral plaques are picked and purified, and the recombinant phenotype is verified. The newly isolated recombinant virus can then be amplified and used to infect insect cell cultures to produce large amounts of the desired protein.

Fusion proteins of the present invention may also be produced using transgenic plants and animals. For example, sheep and goats can make the fusion protein in their milk. Or tobacco plants can include the fusion protein in their leaves. Both transgenic plant and animal production of proteins comprises adding a new gene coding the fusion protein into the genome of the organism. Not only can the transgenic organism produce a new protein, but it can also pass this ability onto its offspring.

Secretory Signal Sequences

The terms “secretory signal sequence” or “signal sequence” or “secretion leader sequence” are used interchangeably and are described, for example in U.S. Pat. No. 6,291,212 and U.S. Pat. No. 5,547,871, both of which are herein incorporated by reference in their entirety. Secretory signal sequences or signal sequences or secretion leader sequences encode secretory peptides. A secretory peptide is an amino acid sequence that acts to direct the secretion of a mature polypeptide or protein from a cell. Secretory peptides are generally characterized by a core of hydrophobic amino acids and are typically (but not exclusively) found at the amino termini of newly synthesized proteins. Very often the secretory peptide is cleaved from the mature protein during secretion. Secretory peptides may contain processing sites that allow cleavage of the signal peptide from the mature protein as it passes through the secretory pathway. Processing sites may be encoded within the signal peptide or may be added to the signal peptide by, for example, in vitro mutagenesis.

Secretory peptides may be used to direct the secretion of fusion proteins of the invention. One such secretory peptide that may be used in combination with other secretory peptides is the alpha mating factor leader sequence. Secretory signal sequences or signal sequences or secretion leader sequences are required for a complex series of post-translational processing steps which result in secretion of a protein. If an intact signal sequence is present, the protein being expressed enters the lumen of the rough endoplasmic reticulum and is then transported through the Golgi apparatus to secretory vesicles and is finally transported out of the cell. Generally, the signal sequence immediately follows the initiation codon and encodes a signal peptide at the amino-terminal end of the protein to be secreted. In most cases, the signal sequence is cleaved off by a specific protease, called a signal peptidase. Preferred signal sequences, such as the Tf or human Tf signal sequence, improve the processing and export efficiency of recombinant protein expression using viral, mammalian or yeast expression vectors.

Detection of Tf Fusion Proteins

Assays for detection of biologically active fusion protein may include Western transfer, protein blot or colony filter as well as activity based assays that detect the fusion protein comprising an EPM peptide and a second peptide or protein. A Western transfer filter may be prepared using the method described by Towbin et al. (Proc. Natl. Acad. Sci. USA 76: 4350-4354, 1979). Briefly, samples are electrophoresed in a sodium dodecylsulfate polyacrylamide gel. The proteins in the gel are electrophoretically transferred to nitrocellulose paper. Protein blot filters may be prepared by filtering supernatant samples or concentrates through nitrocellulose filters using, for example, a Minifold (Schleicher & Schuell, Keene, N.H.). Colony filters may be prepared by growing colonies on a nitrocellulose filter that has been laid across an appropriate growth medium. In this method, a solid medium is preferred. The cells are allowed to grow on the filters for at least 12 hours. The cells are removed from the filters by washing with an appropriate buffer that does not remove the proteins bound to the filters. A preferred buffer comprises 25 mM Tris-base, 19 mM glycine, pH 8.3, 20% methanol.

Fusion proteins of the invention may be labeled with a radioisotope or other imaging agent and used for in vivo diagnostic purposes. Preferred radioisotope imaging agents include iodine-125 and technetium-99, with technetium-99 being particularly preferred. Methods for producing protein-isotope conjugates are well known in the art, and are described by, for example, Eckelman et al. (U.S. Pat. No. 4,652,440), Parker et al. (WO 87/05030) and Wilber et al. (EP 203,764). Alternatively, the fusion proteins may be bound to spin label enhancers and used for magnetic resonance (MR) imaging. Suitable spin label enhancers include stable, sterically hindered, free radical compounds such as nitroxides. Methods for labeling ligands for MR imaging are disclosed by, for example, Coffman et al. (U.S. Pat. No. 4,656,026).

Detection of a fusion protein of the present invention can be facilitated by coupling (i.e., physically linking) the EPM peptide to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

In one embodiment where one is assaying for the ability of a fusion protein of the invention to bind or compete with an antigen for binding to an antibody, various immunoassays known in the art can be used, including but not limited to, competitive and non-competitive assay systems using techniques such as radioimmunoassays, ELISA (enzyme linked immunosorbent assay), sandwich immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays), complement fixation assays, immunofluorescence assays, protein A assays, and immunoelectrophoresis assays, etc. In one embodiment, the binding of the fusion protein is detected by detecting a label on the fusion protein. In another embodiment, the fusion protein is detected by detecting binding of a secondary antibody or reagent that interacts with the fusion protein. In a further embodiment, the secondary antibody or reagent is labeled. Many means are known in the art for detecting binding in an immunoassay and are within the scope of the present invention.

Fusion proteins of the invention may also be detected by assaying for the activity of the EPM peptide moiety. Specifically, fusion proteins of the invention may be assayed for functional activity (e.g., biological activity or therapeutic activity) using assays known to one of ordinary skill in the art. Additionally, one of skill in the art may routinely assay fragments of an EPM peptide corresponding to a therapeutic protein portion of a fusion protein of the invention, for activity using well-known assays. Further, one of skill in the art may routinely assay fragments of a modified transferrin protein for activity using assays known in the art.

For example, in one embodiment where one is assaying for the ability of a fusion protein of the invention to bind or compete with an EPM peptide for binding to an anti-EMP-1 antibody and/or anti-second peptide antibody, various immunoassays known in the art can be used, including but not limited to, competitive and non-competitive assay systems using techniques such as radioimmunoassays, ELISA (enzyme linked immunosorbent assay), sandwich immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

In a further embodiment, where a binding partner (e.g., a receptor or a ligand) of an EPM peptide is identified, binding to that binding partner by a fusion protein containing that EPM peptide as the therapeutic protein portion of the fusion can be assayed, e.g., by means well-known in the art, such as, for example, reducing and non-reducing gel chromatography, protein affinity chromatography, and affinity blotting. Other methods will be known to the skilled artisan and are within the scope of the invention.

Isolation/Purification of Fusion Proteins

Secreted, biologically active, fusion proteins may be isolated from the medium of host cells grown under conditions that allow the secretion of the biologically active fusion proteins. The cell material is removed from the culture medium, and the biologically active fusion proteins are isolated using isolation techniques known in the art. Suitable isolation techniques include precipitation and fractionation by a variety of chromatographic methods, including gel filtration, ion exchange chromatography and affinity chromatography.

A particularly preferred purification method is affinity chromatography on an iron binding or metal chelating column or an immunoaffinity chromatography using an antibody directed against the transferrin or EPM peptide of the polypeptide fusion. The antibody is preferably immobilized or attached to a solid support or substrate. A particularly preferred substrate is CNBr-activated Sepharose (Pharmacia LKB Technologies, Inc., Piscataway, N.J.). By this method, the medium is combined with the antibody/substrate under conditions that will allow binding to occur. The complex may be washed to remove unbound material, and the fusion protein is released or eluted through the use of conditions unfavorable to complex formation. Particularly useful methods of elution include changes in pH, wherein the immobilized antibody has a high affinity for the fusion protein at a first pH and a reduced affinity at a second (higher or lower) pH; changes in concentration of certain chaotropic agents; or through the use of detergents.

Delivery of a Fusion Protein to the Inside of a Cell and/or Across the Blood Brain Barrier (BBB)

Within the scope of the invention, the fusion proteins may be used as a carrier to deliver an EPM peptide or additional therapeutic complexed to the fusion protein to the inside of a cell or across the blood brain barrier or other barriers including across the cell membrane of any cell type that naturally or engineered to express a corresponding receptor. In these embodiments, the fusion protein will typically be engineered or modified to inhibit, prevent or remove glycosylation to extend the serum half-life of the fusion protein and/or EPM peptide portion. The addition of a targeting peptide is specifically contemplated to further target the fusion protein to a particular cell type, e.g., a cancer cell.

Therapeutic/Prophylactic Administration and Compositions

The fusion proteins (or an EPM peptide) of the invention are administered to achieve efficacious levels in target tissues. Thus, the fusion proteins of the invention may be administered by any number of routes, including, but not limited to, topical, dermal, subdermal, transdermal, parenteral, oral, rectal, or by other means including surgical implantation of an oligonucleotide or ribozyme containing pump or other slow release formulation. The fusion proteins are usually employed in the form of pharmaceutical compositions along with a suitable pharmaceutical carrier.

Due to the activity of the fusion proteins of the invention, they are useful in veterinary and human medicine. As described above, the compositions of the invention are useful for the treatment or prevention of various disorders including, but not limited to, anemia, beta-thalassemia, cystic fibrosis, pregnancy and menstrual disorders, early anemia of prematurity, spinal cord injury, acute blood loss, aging, neoplastic disease states associated with abnormal erythropoiesis, renal insufficiency, diabetes, multiple sclerosis, asthma, HCV or HIV infections, hypertension, hypercholesterolemia, arterial scherosis, arthritis, and Alzheimer's disease, chronic or recurrent diseases including, but not limited to, viral disease or infections, cancer, a metabolic diseases, obesity, autoimmune diseases, inflammatory diseases, allergy, graft-vs.-host disease, systemic microbial infection, cardiovascular disease, psychosis, genetic diseases, neurodegenerative diseases, disorders of hematopoietic cells, diseases of the endocrine system or reproductive systems, gastrointestinal diseases.

The invention provides methods of treatment and prophylaxis by administration to a patient of a therapeutically effective amount of a composition comprising a fusion protein of the invention. The patient is an animal, including, but not limited, to an animal such a cow, horse, sheep, pig, chicken, turkey, quail, cat, dog, mouse, rat, rabbit, guinea pig, etc., and is more preferably a mammal, and most preferably a human.

The compositions of the invention may be administered by any convenient route, for example, orally, topically, by intravenous infusion or bolus injection, by absorption through epithelial or mucocutaneous linings (e.g., oral mucosa, rectal and intestinal mucosa, etc.) and may be administered together with another biologically active agent. The compositions of the invention are preferably administered orally. (See, e.g., section 5.17.1 below). Administration can be systemic or local. Various delivery systems are known, for example, encapsulation in liposomes, microparticles, microcapsules, capsules, etc., and can be used to administer a composition of the invention. In certain embodiments, more than one composition of the invention is administered to a patient. Methods of administration include, but are not limited to, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, scalp, or skin. The preferred mode of administration is left to the discretion of the practitioner, and will depend in-part upon the site of the medical condition. In most instances, administration will result in the release of the composition of the invention for maximum uptake by a cell.

In specific embodiments, it may be desirable to administer one or more compositions of the invention locally to the area in need of treatment. This may be achieved, for example, and not by way of limitation, by topical application (e.g., as a cream); by local infusion during surgery (e.g., in conjunction with a wound dressing after surgery); by injection; by means of a catheter; by means of a suppository; or by means of an implant, said implant being of a porous, non-porous, or gelatinous material, including membranes, such as sialastic membranes, or fibers. In one embodiment, administration can be by direct injection at the site (or former site) of an atherosclerotic plaque tissue.

In another embodiment, the composition is prepared in a form suitable for administration directly or indirectly to surface areas of the body for direct application to affected areas. This formulation includes, but is not limited to, anti-drying agents (e.g., pantethine), penetration enhancers (e.g., dimethyl isosorbide), accelerants (e.g., isopropylmyristate) or other common additives that are known in the industry and used for topical applications (e.g., glycerin, propylene glycol, polyethylene glycols, ethyl alcohol, liposomes, lipids, oils, creams, or emollients).

Most drugs are not able to cross the stratum corneum. However, enhanced penetration can be achieved using a class of compounds known collectively as “penetration enhancers.” Alcohols, sulphoxides, fatty acids, esters, Azone, pyrrolidones, urea and polyoles are just some of the members of this class of compounds (Kalbitz et al., 1996). The objectives of these penetration enhancers are to change the solubility and diffusivity of the drug in the stratum corneum, thus some modulate their effects through the lipid pathway while others modify diffusion via the polar pathway.

To further improve the effectiveness of topical formulations, which deliver the compositions across the stratum corneum, phosphorothioate oligonucleotides may be used. Phosphorothioate-modified oligonucleotides are used since these modifications are known to exhibit significant improvement in the biological half-life of the oligonucleotides when compared to unmodified oligonucleotides. Typically, phosphorothioate-modified oligonucleotides exhibit the same characteristics of naturally occurring DNA molecules. Both natural and phosphorothioate-based DNA oligonucleotides of the same length are approximately the same size, form the same secondary and tertiary structures and possess a large net negative charge with one negative charge at each inter-nucleoside linkage. However, phosphorothioate-modified oligonucleotides have greater resistance to nucleolytic degradation because of the presence of a sulfur atom that is substituted for one of the non-bridging oxygen atoms of the phosphodiester inter-nucleoside linkages.

Addition of various concentrations of the enhancer glycerin has been shown to enhance the penetration of cyclosporin (Nakashima et al., 1996). The use of terpene-based penetration enhancers with aqueous propylene glycol have also shown the capacity to enhance topical delivery rates of 5-fluorouracil (Yamane et al., 1995). 5-fluorouracil, 5-FU, is a model compound for examining the characteristics of hydrophilic compounds in skin permeation studies. Thus, the addition of terpenes in polylene glycol (up to 80%) was able to enhance the flux rate into skin.

Dimethyl isosorbide (DMI) is another penetration enhancer that has shown promise for pharmaceutical formulations. DMI is a water-miscible liquid with a relatively low viscosity (Zia et al., 1991). DMI undergoes complexation with water and polylene glycol but not polyethylene glycol. It is the ability for DMI to complex with water that provides the vehicle with the capacity to enhance the penetration of various steroids. Maximum effects were seen at a DMI:water ratio of 1:2. Evidence in the literature suggests that the effect of pH on DMI is an important consideration when using DMI in various formulations (Brisaert et al., 1996).

Pulmonary administration can also be employed, (e.g., by use of an inhaler or nebulizer), and formulation with an aerosolizing agent, or via perfusion in a fluorocarbon or synthetic pulmonary surfactant. In certain embodiments, the compounds of the invention can be formulated as a suppository, with traditional binders and vehicles such as triglycerides.

In another embodiment, the compositions of the invention can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the compositions of the invention can be delivered in a controlled release system. In one embodiment, a pump may be used (see Langer, supra; Sefton, 1987, CRC Crit. Ref. Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of the target area to be treated, (e.g., the liver), thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

The present compositions will contain a therapeutically effective amount of a fusion protein of the invention, optionally with an additional therapeutic, preferably in purified form, together with a suitable amount of a pharmaceutically acceptable vehicle so as to provide the form for proper administration to the patient.

The term “vehicle” refers to a diluent, adjuvant, excipient, or carrier with which a composition of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the compositions of the invention and pharmaceutically acceptable vehicles are preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents. The preferred range of concentrations for the above composition of an effective delivery vehicle for nucleic acid-based compounds are as follows: ethyl alcohol 15-40%; propylene glycol 0.5-5.0%; glycerin 0.5-5.0%; dimethyl isosorbide 0.1-2.0%; polyethylene glycol ester (as Laureth-4) 0.1-2.0%; disodium EDTA 0.01-0.5%; pantethine 0.01-0.2%, divalent cation (copper, magnesium, manganese, zinc, copper litnium, etc.) 0.01-2% and water to 100%.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in “Remington's Pharmaceutical Sciences” by E. W. Martin.

In an illustrative embodiment, the compositions of the invention are formulated in accordance with routine procedures as a pharmaceutical composition adapted for intravenous administration to human beings. Typically, compositions of the invention for intravenous administration are solutions in sterile isotonic aqueous buffer. Where necessary, the compositions may also include a solubilizing agent. Compositions for intravenous administration may optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition of the invention is to be administered by intravenous infusion, it can be dispensed, for example, with an infusion bottle containing sterile pharmaceutical grade water or saline. Where the compound of the invention is administered by injection, an ampoule of sterile water for injection or saline can be provided so that the ingredients may be mixed prior to administration.

Compositions of the invention for oral delivery may be in the form of tablets, lozenges, aqueous or oily suspensions, granules, powders, emulsions, capsules, syrups, or elixirs. Compounds and compositions of the invention for oral delivery can also be formulated in foods and food mixes. Orally administered compositions may contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compositions of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

The amount of a composition of the invention that will be effective in the treatment of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. However, suitable dosage ranges for oral administration are generally about 0.01 nanomoles (“nmol”) to 200 millimoles (“mmol”) of a fusion protein of the invention per kilogram body weight. In specific preferred embodiments of the invention, the oral dose is 0.01 nmol to 70 mmol per kilogram body weight, more preferably 0.1 nmol to 50 mmol per kilogram body weight, more preferably 0.5 nmol to 20 mmol per kilogram body weight, and yet more preferably 1 nmol to 10 mmol per kilogram body weight. In a most preferred embodiment, the oral dose is 5 nmol of a composition of the invention per kilogram body weight. The dosage amounts described herein refer to total amounts administered; that is, if more than one composition of the invention is administered, the preferred dosages correspond to the total amount of the compounds of the invention administered. Oral compositions preferably contain 10% to 95% active ingredient by weight.

Suitable dosage ranges for intravenous (i.v.) administration are 0.01 nmol to 100 mmol per kilogram body weight, 0.1 nmol to 35 mmol per kilogram body weight, and 1 nmol to 10 mmol per kilogram body weight. Suitable dosage ranges for intranasal administration are generally about 0.01 nmol/kg body weight to 1 mmol/kg body weight. Suppositories generally contain 0.01 nmol to 50 mmol of a composition of the invention per kilogram body weight and comprise active ingredient in the range of 0.5% to 10% by weight. Recommended dosages for intradermal, intramuscular, intraperitoneal, subcutaneous, epidural, sublingual, intracerebral, intravaginal, transdermal administration or administration by inhalation are in the range of 0.001 nmol to 200 mmol per kilogram of body weight. Suitable doses of the compounds of the invention for topical administration are in the range of 0.001 nmol to 1 mmol, depending on the area to which the compound is administered. Effective doses may be extrapolated from dose-response curves derived from in vitro or animal model test systems. Such animal models and systems are well known in the art.

In the case of parenteral administration (e.g., for the treatment of, for example, benign prostatic hyperplasia), the compositions of the invention may be encapsulated in a liposome “envelope” that is coupled to an antibody directed against human prostate-specific proteins so as to provide target cell selectivity. The specific nature of the formulation is determined by the desired route of administration, e.g., topical, parenteral, oral, rectal, surgical implantation or by other means of local (intraprostatic) delivery. The dosage is determined for the route of administration. The amount of oligonucleotide or ribozyme in the composition can range from about 0.01 to 99% by weight of the composition. Direct treatment of the prostate may involve the perineal administration of a suitable preparation of at least one anti-sense oligonucleotide under echographic control. The injection may be made in either the zone of hyperplasia or in the external gland. A similar approach has been reported for the treatment of chronic prostatitis through the intraprostatic injection of antibiotics (Jimenez et al., 1988). In these studies transitory post-injection hemospermia together with pain during or after injection were the sole adverse effects observed with this therapy.

Compositions for rectal administration are prepared with any of the usual pharmaceutical excipients, including for example, binders, lubricants and disintegrating agents. The composition may also include cell penetration enhancers, such as aliphatic sulfoxides. In a preferred embodiment, the composition of the present invention is in the form of a suppository.

The invention also provides pharmaceutical packs or kits comprising one or more containers filled with one or more compounds of the invention, as discussed in section 5.20. Optionally associated with such container(s) can be a notice in the form prescribed by a governmental agency regulating the manufacture, use or sale of pharmaceuticals or biological products, which notice reflects approval by the agency of manufacture, use or sale for human administration.

The compounds of the invention are preferably assayed in vitro and in vivo, for the desired therapeutic or prophylactic activity, prior to use in humans. For example, in vitro assays can be used to determine whether administration of a specific composition of the invention or a combination of compositions of the invention is preferred for treating or ameliorating a disease or disorder as described herein. The compositions of the invention may also be demonstrated to be effective and safe using animal model systems.

Oral Administration

In a particular embodiment, the fusion proteins may be formulated for oral delivery. In particular, certain fusion proteins of the invention that are used to treat certain classes of a diseases or medical conditions may be particularly amenable for oral formulation and delivery. Such classes of diseases or conditions include, but are not limited to, acute, chronic and recurrent diseases. Chronic or recurrent diseases include, but are not limited to, viral disease or infections, cancer, a metabolic diseases, obesity, autoimmune diseases, inflammatory diseases, allergy, graft-vs.-host disease, systemic microbial infection, anemia, cardiovascular disease, psychosis, genetic diseases, neurodegenerative diseases, disorders of hematopoietic cells, diseases of the endocrine system or reproductive systems, gastrointestinal diseases. Examples of these classes of disease include diabetes, multiple sclerosis, asthma, HCV or HIV infections, hypertension, hypercholesterolemia, arterial scherosis, arthritis, and Alzheimer's disease. In many chronic diseases, oral formulations of fusion proteins of the invention and methods of administration are particularly useful because they allow long-term patient care and therapy via home oral administration without reliance on injectable treatment or drug protocols.

Oral formulations and delivery methods comprising fusion proteins of the invention take advantage of, in part, various receptor mediated transcytosis across the gastrointestinal (GI) epithelium. For example, the transferrin receptor is found at a very high density in the human GI epithelium, transferrin is highly resistant to tryptic and chymotryptic digestion and Tf chemical conjugates have been used to successfully deliver proteins and peptides across the GI epithelium (Xia et al., (2000) J. Pharmacol. Experiment. Therap., 295:594-600; Xia et al. (2001) Pharmaceutical Res., 18(2):191-195; and Shah et al. (1996) J. Pharmaceutical Sci., 85(12):1306-1311, all of which are herein incorporated by reference in their entirety). Once transported across the GI epithelium, fusion proteins of the invention exhibit extended half-life in serum, that is, the EPM peptide attached or inserted into a second peptide or protein exhibit an extended serum half-life compared to the EPM peptide in its non-fused state. Fusion proteins of the invention that are not amenable to oral administration due to, for example, digestion by gastric enzymes can be administered by other techniques described herein or known to those of ordinary skill in the art.

Oral formulations of fusion proteins of the invention may be prepared so that they are suitable for transport to the GI epithelium and protection of the fusion protein component and other active components in the stomach. Such formulations may include carrier and dispersant components and may be in any suitable form, including aerosols (for oral or pulmonary delivery), syrups, elixirs, tablets, including chewable tablets, hard or soft capsules, troches, lozenges, aqueous or oily suspensions, emulsions, cachets or pellets granulates, and dispersible powders. Preferably, fusion protein formulations are employed in solid dosage forms suitable for simple, and preferably oral, administration of precise dosages. Solid dosage forms for oral administration are preferably tablets, capsules, or the like.

For oral administration in the form of a tablet or capsule, care should be taken to ensure that the composition enables sufficient active ingredient to be absorbed by the host to produce an effective response. Thus, for example, the amount of fusion protein may be increased over that theoretically required or other known measures such as coating or encapsulation may be taken to protect the polypeptides from enzymatic action in the stomach.

Traditionally, peptide and protein drugs have been administered by injection because of the poor bioavailability when administered non-parenterally, and in particular orally. These drugs are prone to chemical and conformational instability and are often degraded by the acidic conditions in the stomach, as well as by enzymes in the stomach and gastrointestinal tract. In response to these delivery problems, certain technologies for oral delivery have been developed, such as encapsulation in nanoparticles composed of polymers with a hydrophobic backbone and hydrophilic branches as drug carriers, encapsulation in microparticles, insertion into liposomes in emulsions, and conjugation to other molecules. All of which may be used with the fusion proteins of the present invention.

Examples of nanoparticles include mucoadhesive nanoparticles coated with chitosan and Carbopol (Takeuchi et al., Adv. Drug Deliv. Rev. 47(1):39-54, 2001) and nanoparticles containing charged combination polyesters, poly(2-sulfobutyl-vinyl alcohol) and poly(D,L-lactic-co-glycolic acid) (Jung et al., Eur. J. Pharm. Biopharm. 50(1):147-160,2000). Nanoparticles containing surface polymers with poly-N-isopropylacrylamide regions and cationic poly-vinylamine groups showed improved absorption of salmon calcitonin when administered orally to rats.

Drug delivery particles composed of alginate and pectin, strengthened with polylysine, are relatively acid and base resistant and can be used as a carrier for drugs. These particles combine the advantages of bioadhesion, enhanced absorption and sustained release (Liu et al., J. Pharm. Pharmacol. 51(2):141-149, 1999).

Additionally, lipoamino acid groups and liposaccharide groups conjugated to the N- and C-termini of peptides such as synthetic somatostatin, creating an amphipathic surfactant, were shown to produce a composition that retained biological activity (Toth et al., J. Med. Chem. 42(19):4010-4013, 1999).

Examples of other peptide delivery technologies include carbopol-coated mucoadhesive emulsions containing the peptide of interest and either nitroso-N-acetyl-D,L-penicillamine and carbolpol or taurocholate and carbopol. These were shown to be effective when orally administered to rats to reduce serum calcium concentrations (Ogiso et al., Biol. Pharm. Bull. 24(6):656-661, 2001). Phosphatidylethanol, derived from phosphatidylcholine, was used to prepare liposomes containing phosphatidylethanol as a carrier of insulin. These liposomes, when administered orally to rats, were shown to be active (Kisel et al., Int. J. Pharm. 216(1-2):105-114, 2001).

Insulin has also been formulated in poly(vinyl alcohol)-gel spheres containing insulin and a protease inhibitor, such as aprotinin or bacitracin. The glucose-lowering properties of these gel spheres have been demonstrated in rats, where insulin is released largely in the lower intestine (Kimura et al., Biol. Pharm. Bull. 19(6):897-900, 1996.

Oral delivery of insulin has also been studied using nanoparticles made of poly(alkyl cyanoacrylate) that were dispersed with a surfactant in an oily phase (Damge et al., J. Pharm. Sci. 86(12):1403-1409, 1997) and using calcium alginate beads coated with chitosan (Onal et al., Artif. Cells Blood Substit. Immobil. Biotechnol. 30(3):229-237, 2002).

In other methods, the N- and C-termini of a peptide are linked to polyethylene glycol and then to allyl chains to form conjugates with improved resistance to enzymatic degradation and improved diffusion through the GI wall (www.nobexcorp.com).

BioPORTER® is a cationic lipid mixture, which interacts non-covalently with peptides to create a protective coating or layer. The peptide-lipid complex can fuse to the plasma membrane of cells, and the peptides are internalized into the cells (www.genetherapysystems.com).

In a process using liposomes as a starting material, cochleate-shaped particles have been developed as a pharmaceutical vehicle. A peptide is added to a suspension of liposomes containing mainly negatively charged lipids. The addition of calcium causes the collapse and fusion of the liposomes into large sheets composed of lipid bilayers, which then spontaneously roll up or stack into cochleates (U.S. Pat. No. 5,840,707; http://www.biodeliverysciences.com).

Compositions comprising fusion protein intended for oral use may be prepared according to any method known to the art for the manufacture of pharmaceutical compositions and such compositions may contain one or more agents including, but not limited to, sweetening agents in order to provide a pharmaceutically elegant and palatable preparation. For example, to prepare orally deliverable tablets, a fusion protein is mixed with at least one pharmaceutical excipient, and the solid formulation is compressed to form a tablet according to known methods, for delivery to the gastrointestinal tract. The tablet composition is typically formulated with additives, (e.g., a saccharide or cellulose carrier) a binder such as starch paste or methyl cellulose, a filler, a disintegrator, or other additives typically usually used in the manufacture of medical preparations. To prepare orally deliverable capsules, DHEA is mixed with at least one pharmaceutical excipient, and the solid formulation is placed in a capsular container suitable for delivery to the gastrointestinal tract. Compositions comprising a fusion protein may be prepared as described generally in Remington's Pharmaceutical Sciences, 18th Ed. 1990 (Mack Publishing Co. Easton Pa. 18042) at Chapter 89, which is herein incorporated by reference.

As described above, many of the oral formulations of the invention may contain inert ingredients, which allow for protection against the stomach environment, and release of the biologically active material in the intestine. Such formulations, or enteric coatings, are well known in the art. For example, tablets containing a fusion protein in admixture with non-toxic pharmaceutically acceptable excipients, which are suitable for manufacture of tablets may be used. These excipients may be inert diluents, such as calcium carbonate, sodium carbonate, lactose, calcium phosphate or sodium phosphate; granulating and disintegrating agents, for example, maize starch, gelatin or acacia, and lubricating agents, for example, magnesium stearate, stearic acid, or talc.

The tablets may be uncoated or they may be coated with known techniques to delay disintegration and absorption in the gastrointestinal track and thereby provide a sustained action over a longer period of time. For example, a time delay material such as glyceryl monostearate or glyceryl distearate alone or with a wax may be employed.

Formulations for oral use may also be presented as hard gelatin capsules, wherein the active ingredient is mixed with an inert solid diluent, for example, calcium carbonate, calcium phosphate, or kaolin or as soft gelatin capsules wherein the active ingredient is mixed with an aqueous or an oil medium, for example, arachis oil, peanut oil, liquid paraffin or olive oil.

Aqueous suspensions may contain a fusion protein in the admixture with excipients suitable for the manufacture of aqueous suspensions. Such excipients are suspending agents, for example, sodium carboxymethylcellulose, methylcellulose, hydroxypropylmethylcellulose, sodium alginate, polyvinylpyrrolidone, gum tragacanth and gum acacia; dispersing or wetting agents may be a naturally occurring phosphatide, for example, lecithin, or condensation products of an alkylene oxide with fatty acids, for example, polyoxyethylene stearate, or condensation products of ethylene oxide with long chain aliphatic alcohols, for example, heptadecylethyloxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate. The aqueous suspensions may also contain one or more preservatives for example, ethyl or n-propyl p-hydroxybenzoate, one or more coloring agents, one or more flavoring agents and one or more sweetening agents such as sucrose or saccharin.

Oily suspensions may be formulated by suspending the active ingredient in a vegetable oil, for example, arachis oil, olive oil, sesame oil or coconut oil, or in a mineral oil such as liquid paraffin. The oil suspensions may contain a thickening agent, for example, beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient and admixture with dispersing or wetting agent, suspending agent and one or more preservatives. Suitable dispersing or wetting agents and suspending agents are exemplified by those already mentioned above. Additional excipients, for example, sweetening, flavoring and coloring agents, may also be present.

The pharmaceutical compositions containing fusion protein may also be in the form of oil-in-water emulsions. The oil phase may be a vegetable oil, for example, olive oil or arachis oil, or a mineral oil for example, gum acacia or gum tragacanth, naturally-occurring phosphotides, for example soybean lecithin, and esters or partial esters derived from fatty acids and hexitol anhydrides, for example, sorbitan monooleate, and condensation products of the same partial esters with ethylene oxide, for example, polyoxyethylene sorbitan monooleate. The emulsions may also contain sweetening and flavoring agents.

Syrups and elixirs containing fusion protein may be formulated with sweetening agents, for example, glycerol, sorbitol or sucrose. Such formulations may also contain a demulcent, a preservative and flavoring and coloring agents. The pharmaceutical compositions may be in the form of a sterile injectable preparation, for example, as a sterile injectable aqueous or oleaginous suspension. This suspension may be formulated according to the known art using those suitable dispersing or wetting agents and suspending agents, which have been mentioned above. The sterile injectable preparations may also be a sterile injectable solution or suspension in a non-toxic parenterally-acceptable diluent or solvate, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this period any bland fixed oil may be employed including synthetic mono- or diglycerides. In addition, fatty acids such as oleic acid find use in the preparation of injectables.

Pharmaceutical compositions may also be formulated for oral delivery using polyester microspheres, zein microspheres, proteinoid microspheres, polycyanoacrylate microspheres, and lipid-based systems (see, for example, DiBase and Morrel, Oral Delivery of Microencapsulated Proteins, in Protein Delivery: Physical Systems, Sanders and Hendren (eds.), pages 255-288 (Plenum Press 1997)).

The proportion of pharmaceutically active fusion protein to carrier and/or other substances may vary from about 0.5 to about 100 wt. % (weight percent). For oral use, the pharmaceutical formulation will generally contain from about 5 to about 100% by weight of the active material. For other uses, the formulation will generally have from about 0.5 to about 50 wt. % of the active material.

Fusion protein formulations employed in the invention provide an therapeutically effective amount of the fusion protein upon administration to an individual to ameliorate a symptom of a disease.

The fusion protein composition of the invention may be, though not necessarily, administered daily, in an effective amount to ameliorate a symptom. Generally, the total daily dosage will be at least about 1 mg, preferably at least about 5, preferably at least about 10 mg, preferably at least about 25 mg, preferably at least about 50 mg, preferably at least about 100 mg, and more preferably at least about 200 mg, and preferably not more than 500 mg per day, administered orally. In another embodiment, fusion protein composition of the invention may be, though not necessarily, administered daily more than once daily (e.g., 4 capsules or tablets, each containing 50 mg fusion protein every six hours). Capsules or tablets for oral delivery can conveniently contain up to a full daily oral dose, for example, 200 mg or more.

In a particularly preferred embodiment, oral pharmaceutical compositions comprising fusion protein are formulated in buffered liquid form which is then encapsulated into soft or hard-coated gelatin capsules which are then coated with an appropriate enteric coating. For the oral pharmaceutical compositions of the invention, the location of release may be anywhere in the GI system, including the small intestine (the duodenum, the jejunum, or the ileum), or the large intestine.

In other embodiments, oral compositions of the invention are formulated to slowly release the active ingredients, including the fusion proteins of the invention, in the GI system using known delayed release formulations.

In some pharmaceutical formulations of the invention, the fusion protein is engineered to contain a cleavage site between the EPM peptide and the second peptide moiety. Such cleavable sites or linkers are known in the art.

Pharmaceutical compositions of the invention and methods of the invention may include the addition of a transcytosis enhancer to facilitate transfer of the fusion protein across the GI epithelium. Such enhancers are known in the art. See Xia et al., (2000) J Pharmacol. Experiment. Therap., 295:594-600; and Xia et al. (2001) Pharmaceutical Res., 18(2):191-195.

In preferred embodiments of the invention, oral pharmaceutical formulations include fusion proteins comprising a second peptide moiety exhibiting reduced or no glycosylation fused at the N terminal end to an EPM peptide as described above. Such pharmaceutical compositions may be used to treat glucose imbalance disorders such as diabetes by oral administration of the pharmaceutical composition comprising an effective dose of fusion protein.

The effective dose of fusion protein may be measured in a numbers of ways, including dosages calculated to alleviate symptoms associated with a specific disease state in a patient, such as the symptoms of diabetes. In other formulations, dosages are calculated to comprise an effective amount of fusion protein to induce a detectable change in blood glucose levels in the patient. Such detectable changes in blood glucose may include a decrease in blood glucose levels of between about 1% and 90%, or between about 5% and about 80%. These decreases in blood glucose levels will be dependent on the disease condition being treated and pharmaceutical compositions or methods of administration may be modified to achieve the desired result for each patient. In other instances, the pharmaceutical compositions are formulated and methods of administration modified to detect an increase in the activity level of the EPM peptide in the patient. Such formulations and methods may deliver between about 1 pg to about 100 mg/kg body weight of fusion protein, about 100 ng to about 100 μg/kg body weight of fusion protein, about 100 μg/kg to about 100 mg/kg body weight of fusion protein, about 1 μg to about 1 g of fusion protein, about 10 μg to about 100 mg of fusion protein or about 1 mg to about 50 mg of fusion protein. Formulations may also be calculated using a unit measurement of modified EMP-1 activity. The measurements by weight or activity can be calculated using known standards for each EPM peptide fused to Tf.

The invention also includes methods of orally administering the pharmaceutical compositions of the invention. Such methods may include, but are not limited to, steps of orally administering the compositions by the patient or a caregiver. Such administration steps may include administration on intervals such as once or twice per day depending on the fusion protein, disease or patient condition or individual patient. Such methods also include the administration of various dosages of the individual fusion protein. For instance, the initial dosage of a pharmaceutical composition may be at a higher level to induce a desired effect, such as reduction in blood glucose levels. Subsequent dosages may then be decreased once a desired effect is achieved. These changes or modifications to administration protocols may be done by the attending physician or health care worker. In some instances, the changes in the administration protocol may be done by the individual patient, such as when a patient is monitoring blood glucose levels and administering a fusion protein oral composition of the invention.

The invention also includes methods of producing oral compositions or medicant compositions of the invention comprising formulating a fusion protein of the invention into an orally administerable form. In other instances, the invention includes methods of producing compositions or medicant compositions of the invention comprising formulating a fusion protein of the invention into a form suitable for oral administration.

Moreover, the present invention includes pulmonary delivery of the fusion protein formulations. Pulmonary delivery is particularly promising for the delivery of macromolecules, which are difficult to deliver by other routes of administration. Such pulmonary delivery can be effective both for systemic delivery and for localized delivery to treat diseases of the lungs, since drugs delivered to the lung are readily absorbed through the alveolar region directly into the blood circulation.

The invention provides compositions suitable for forming a drug dispersion for oral inhalation (pulmonary delivery) to treat various conditions or diseases. The fusion protein formulation could be delivered by different approaches such as liquid nebulizers, aerosol-based metered dose inhalers (MDI's), and dry powder dispersion devices. In formulating compositions for pulmonary delivery, pharmaceutically acceptable carriers including surface active agents or surfactants and bulk carriers are commonly added to provide stability, dispersibility, consistency, and/or bulking characteristics to enhance uniform pulmonary delivery of the composition to the subject.

Surface active agents or surfactants promote absorption of polypeptide through mucosal membrane or lining. Useful surface active agents or surfactants include fatty acids and salts thereof, bile salts, phospholipid, or an alkyl saccharide. Examples of fatty acids and salts thereof include sodium, potassium and lysine salts of caprylate (C₈), caprate (C₁₀), laurate (C₁₂) and myristate (C₁₄). Examples of bile salts include cholic acid, chenodeoxycholic acid, glycocholic acid, taurocholic acid, glycochenodeoxycholic acid, taurochenodeoxycholic acid, deoxycholic acid, glycodeoxycholic acid, taurodeoxycholic acid, lithocholic acid, and ursodeoxycholic acid.

Examples of phospholipids include single-chain phospholipids, such as lysophosphatidylcholine, lysophosphatidylglycerol, lysophosphatidylethanolamine, lysophosphatidylinositol and lysophosphatidylserine; or double-chain phospholipids, such as diacylphosphatidylcholines, diacylphosphatidylglycerols, diacylphosphatidylethanolamines, diacylphosphatidylinositols and diacylphosphatidylserines. Examples of alkyl saccharides include alkyl glucosides or alkyl maltosides, such as decyl glucoside and dodecyl maltoside.

Pharmaceutical excipients that are useful as carriers include stabilizers such as human serum albumin (HSA) or recombinant human albumin; bulking agents such as carbohydrates, amino acids and polypeptides; pH adjusters or buffers; salts such as sodium chloride; and the like. These carriers may be in a crystalline or amorphous form or may be a mixture of the two.

Examples of carbohydrates for use as bulking agents include monosaccharides such as galactose, D-mannose, sorbose, and the like; disaccharides, such as lactose, trehalose, and the like; cyclodextrins, such as 2-hydroxypropyl-.beta.-cyclodextrin; and polysaccharides, such as raffinose, maltodextrins, dextrans, and the like; alditols, such as mannitol, xylitol, and the like. Examples of polypeptides for use as bulking agents include aspartame. Amino acids include alanine and glycine, with glycine being preferred.

Additives, which are minor components of the composition, may be included for conformational stability during spray drying and for improving dispersibility of the powder. These additives include hydrophobic amino acids such as tryptophan, tyrosine, leucine, phenylalanine, and the like.

Suitable pH adjusters or buffers include organic salts prepared from organic acids and bases, such as sodium citrate, sodium ascorbate, and the like; sodium citrate is preferred.

The fusion protein compositions for pulmonary delivery may be packaged as unit doses where a therapeutically effective amount of the composition is present in a unit dose receptacle, such as a blister pack, gelatin capsule, or the like. The manufacture of blister packs or gelatin capsules is typically carried out by methods that are generally well known in the packaging art.

U.S. Pat. No. 6,524,557 discloses a pharmaceutical aerosol formulation comprising (a) a HFA propellant; (b) a pharmaceutically active polypeptide dispersible in the propellant; and (c) a surfactant which is a C₈-C₁₆ fatty acid or salt thereof, a bile salt, a phospholipid, or an alkyl saccharide, which surfactant enhances the systemic absorption of the polypeptide in the lower respiratory tract. The invention also provides methods of manufacturing such formulations and the use of such formulations in treating patients.

One approach for the pulmonary delivery of dry powder drugs utilizes a hand-held device with a hand pump for providing a source of pressurized gas. The pressurized gas is abruptly released through a powder dispersion device, such as a venturi nozzle, and the dispersed powder made available for patient inhalation.

Dry powder dispersion devices are described in several patents. U.S. Pat. No. 3,921,637 describes a manual pump with needles for piercing through a single capsule of powdered medicine. The use of multiple receptacle disks or strips of medication is described in European Patent Application No. EP 0 467 172; International Patent Publication Nos. WO 91/02558; and WO 93/09832; U.S. Pat. Nos. 4,627,432; 4,811,731; 5,035,237; 5,048,514; 4,446,862; 5,048,514, and 4,446,862.

The aerosolization of protein therapeutic agents is disclosed in European Patent Application No. EP 0 289 336. Therapeutic aerosol formulations are disclosed in International Patent Publication No. WO 90/09781.

The present invention provides formulating fusion protein for oral inhalation. The formulation comprises fusion protein and suitable pharmaceutical excipients for pulmonary delivery. The present invention also provides administering the fusion protein composition via oral inhalation to subjects in need thereof.

Transdermal Delivery

The present invention also provides formulating fusion proteins for transdermal delivery. Transdermal systems deliver therapeutic formulations through the skin into the bloodstream, making them easy to administer. Passive and active transdermal delivery systems are used to deliver medicines in even concentrations in a way that is painless and results in few adverse side effects. The fusion proteins could be delivered transdermally using microneedles and other means such as a skin patch. Henry et al. discuss a method of mechanically puncturing the skin with microneedles in order to increase the permeability of skin to a test drug (Micromachined needles for the transdermal delivery of drugs, IEEE 11th Annual International Workshop on Micro-Electro-Mechanical Systems (1998), pp. 494-498, which is incorporated herein by reference.

Transgenic Animals

The production of transgenic non-human animals that contain a fusion protein with increased serum half-life increased serum stability or increased bioavailability of the instant invention is contemplated in one embodiment of the invention.

The successful production of transgenic, non-human animals has been described in a number of patents and publications, such as, for example U.S. Pat. No. 6,291,740 (issued Sep. 18, 2001); U.S. Pat. No. 6,281,408 (issued Aug. 28, 2001); and U.S. Pat. No. 6,271,436 (issued Aug. 7, 2001) the contents of which are hereby incorporated by reference in their entireties.

The ability to alter the genetic make-up of animals, such as domesticated mammals including cows, pigs, goats, horses, cattle, and sheep, allows a number of commercial applications. These applications include the production of animals which express large quantities of exogenous proteins in an easily harvested form (e.g., expression into the milk or blood), the production of animals with increased weight gain, feed efficiency, carcass composition, milk production or content, disease resistance and resistance to infection by specific microorganisms and the production of animals having enhanced growth rates or reproductive performance. Animals which contain exogenous DNA sequences in their genome are referred to as transgenic animals.

The most widely used method for the production of transgenic animals is the microinjection of DNA into the pronuclei of fertilized embryos (Wall et al., J. Cell. Biochem. 49:113 [1992]). Other methods for the production of transgenic animals include the infection of embryos with retroviruses or with retroviral vectors. Infection of both pre- and post-implantation mouse embryos with either wild-type or recombinant retroviruses has been reported (Janenich, Proc. Natl. Acad. Sci. USA 73:1260 [1976]; Janenich et al., Cell 24:519 [1981]; Stuhlmann et al., Proc. Natl. Acad. Sci. USA 81:7151 [1984]; Jahner et al., Proc. Natl. Acad Sci. USA 82:6927 [1985]; Van der Putten et al., Proc. Natl. Acad. Sci. USA 82:6148-6152 [1985]; Stewart et al., EMBO J. 6:383-388 [1987]).

An alternative means for infecting embryos with retroviruses is the injection of virus or virus-producing cells into the blastocoele of mouse embryos (Jahner, D. et al., Nature 298:623 [1982]). The introduction of transgenes into the germline of mice has been reported using intrauterine retroviral infection of the midgestation mouse embryo (Jahner et al., supra [1982]). Infection of bovine and ovine embryos with retroviruses or retroviral vectors to create transgenic animals has been reported. These protocols involve the micro-injection of retroviral particles or growth arrested (i.e., mitomycin C-treated) cells which shed retroviral particles into the perivitelline space of fertilized eggs or early embryos (PCT International Application WO 90/08832 [1990]; and Haskell and Bowen, Mol. Reprod. Dev., 40:386 [1995]. PCT International Application WO 90/08832 describes the injection of wild-type feline leukemia virus B into the perivitelline space of sheep embryos at the 2 to 8 cell stage. Fetuses derived from injected embryos were shown to contain multiple sites of integration.

U.S. Pat. No. 6,291,740 (issued Sep. 18, 2001) describes the production of transgenic animals by the introduction of exogenous DNA into pre-maturation oocytes and mature, unfertilized oocytes (i.e., pre-fertilization oocytes) using retroviral vectors which transduce dividing cells (e.g., vectors derived from murine leukemia virus [MLV]). This patent also describes methods and compositions for cytomegalovirus promoter-driven, as well as mouse mammary tumor LTR expression of various recombinant proteins.

U.S. Pat. No. 6,281,408 (issued Aug. 28, 2001) describes methods for producing transgenic animals using embryonic stem cells. Briefly, the embryonic stem cells are used in a mixed cell co-culture with a morula to generate transgenic animals. Foreign genetic material is introduced into the embryonic stem cells prior to co-culturing by, for example, electroporation, microinjection or retroviral delivery. ES cells transfected in this manner are selected for integrations of the gene via a selection marker such as neomycin.

U.S. Pat. No. 6,271,436 (issued Aug. 7, 2001) describes the production of transgenic animals using methods including isolation of primordial germ cells, culturing these cells to produce primordial germ cell-derived cell lines, transforming both the primordial germ cells and the cultured cell lines, and using these transformed cells and cell lines to generate transgenic animals. The efficiency at which transgenic animals are generated is greatly increased, thereby allowing the use of homologous recombination in producing transgenic non-rodent animal species.

Gene Therapy

The use of fusion proteins for gene therapy, wherein a second peptide domain is joined to an EPM peptide is contemplated in one embodiment of this invention. The fusion proteins with increased serum half-life or serum stability of the instant invention are ideally suited to gene therapy treatments.

The successful use of gene therapy to express a soluble fusion protein has been described. Briefly, gene therapy via injection of an adenovirus vector containing a gene encoding a soluble fusion protein consisting of cytotoxic lymphocyte antibody 4 (CTLA4) and the Fc portion of human immunoglobulin G1 was recently shown in Ijima et al. (Jun. 10, 2001) Human Gene Therapy (United States) 12/9:1063-77. In this application of gene therapy, a murine model of type II collagen-induced arthritis was successfully treated via intraarticular injection of the vector.

Gene therapy is also described in a number of U.S. patents including U.S. Pat. No. 6,225,290 (issued May 1, 2001); U.S. Pat. No. 6,187,305 (issued Feb. 13, 2001); and U.S. Pat. No. 6,140,111 (issued Oct. 31, 2000).

U.S. Pat. No. 6,225,290 provides methods and constructs whereby intestinal epithelial cells of a mammalian subject are genetically altered to operatively incorporate a gene which expresses a protein which has a desired therapeutic effect. Intestinal cell transformation is accomplished by administration of a formulation composed primarily of naked DNA, and the DNA may be administered orally. Oral or other intragastrointestinal routes of administration provide a simple method of administration, while the use of naked nucleic acid avoids the complications associated with use of viral vectors to accomplish gene therapy. The expressed protein is secreted directly into the gastrointestinal tract and/or blood stream to obtain therapeutic blood levels of the protein thereby treating the patient in need of the protein. The transformed intestinal epithelial cells provide short or long term therapeutic cures for diseases associated with a deficiency in a particular protein or which are amenable to treatment by overexpression of a protein.

U.S. Pat. No. 6,187,305 provides methods of gene or DNA targeting in cells of vertebrate, particularly mammalian, origin. Briefly, DNA is introduced into primary or secondary cells of vertebrate origin through homologous recombination or targeting of the DNA, which is introduced into genomic DNA of the primary or secondary cells at a preselected site.

U.S. Pat. No. 6,140,111 (issued Oct. 31, 2000) describes retroviral gene therapy vectors. The disclosed retroviral vectors include an insertion site for genes of interest and are capable of expressing high levels of the protein derived from the genes of interest in a wide variety of transfected cell types. Also disclosed are retroviral vectors lacking a selectable marker, thus rendering them suitable for human gene therapy in the treatment of a variety of disease states without the co-expression of a marker product, such as an antibiotic. These retroviral vectors are especially suited for use in certain packaging cell lines. The ability of retroviral vectors to insert into the genome of mammalian cells has made them particularly promising candidates for use in the genetic therapy of genetic diseases in humans and animals. Genetic therapy typically involves (1) adding new genetic material to patient cells in vivo, or (2) removing patient cells from the body, adding new genetic material to the cells and reintroducing them into the body, i.e., in vitro gene therapy. Discussions of how to perform gene therapy in a variety of cells using retroviral vectors can be found, for example, in U.S. Pat. Nos. 4,868,116, issued Sep. 19, 1989, and 4,980,286, issued Dec. 25, 1990 (epithelial cells), WO 89/07136 published Aug. 10, 1989 (hepatocyte cells), EP 378,576 published Jul. 25, 1990 (fibroblast cells), and WO 89/05345 published Jun. 15, 1989 and WO/90/06997, published Jun. 28, 1990 (endothelial cells), the disclosures of which are incorporated herein by reference.

Kits Containing Fusion Proteins

In a further embodiment, the invention provides kits containing fusion proteins, which can be used, for instance, for the therapeutic or non-therapeutic applications. The kit comprises a container with a label. Suitable containers include, for example, bottles, vials, and test tubes. The containers may be formed from a variety of materials such as glass or plastic. The container holds a composition which includes a fusion protein that is effective for therapeutic or non-therapeutic applications, such as described herein. The active agent in the composition is the EPM peptide. The label on the container indicates that the composition is used for a specific therapy or non-therapeutic application, and may also indicate directions for either in vivo or in vitro use, such as those described above.

The kit of the invention will typically comprise the container described above and one or more other containers comprising materials desirable from a commercial and user standpoint, including buffers, diluents, filters, needles, syringes, and package inserts with instructions for use.

Without further description, it is believed that a person of ordinary skill in the art can, using the preceding description and the following illustrative examples, make and utilize the present invention and practice the claimed methods. For example, a skilled artisan would readily be able to determine the biological activity, both in vitro and in vivo, for the fusion protein constructs of the present invention as compared with the comparable activity of the therapeutic moiety in its unfused state. Similarly, a person skilled in the art could readily determine the serum half life and serum stability of constructs according to the present invention. The following working examples therefore, specifically point out the illustrative embodiments of the invention, and are not to be construed as limiting in any way the remainder of the disclosure.

### EXAMPLES

### Example 1

### EPM Peptide/Transferrin Fusion Protein

EMP-1 (SEQ ID NO: 4) has been shown to mimic EPO activity by causing dimerization of the EPO receptor. The peptide, which is cyclic, has no homology to EPO. To become active, the peptide has to act in concert with another peptide, i.e. as a dimer, such that two copies of the receptor are brought in close enough proximity to form an active complex. As with many peptides, the peptide dimer suffers from short half-life and would benefit from the longevity that fusion to transferrin would give. The invention provides fusion proteins with EPO mimetic activity. As an illustrative example, the fusion protein of the present invention comprises an EPM peptide and modified transferrin (mTf) with increased half-life. The invention also encompasses pharmaceutical compositions of such fusion proteins for the treatment of diseases associated with low or defective red blood cell production.

EPM Peptide Fusions And Insertions

The initial fusions to mTf comprise fusions to the N—, C—, and N- and C-termini of mTf. The individual fusions will bind the receptor but not cause activation of the receptor. The dual fusion, one of which should, preferably, be of a different codon composition than the other to prevent recombination, will enable binding to the receptor and cause activation.

Examination of the N lobe of human Tf (PDB identifier 1A8E) and the full Tf model AAAaoTfwo, generated using the ExPasy Swiss Model Server with the rabbit model 1JNF as template, reveals a number of potential sites for insertion of a peptide, either directly or by replacement of a number of residues. These sites are duplicated by their equivalent sites in the C lobe.

| N₁ | N₂ |
|---|---|
| Asp33 | Ser105 |
| Asn55 | Glu141 |
| Asn75 | Asp166 |
| Asp90 | Gln184 |
| Gly257 | Asp197 |
| Lys280 | Lys217 |
| His289 | Thr231 |
| Ser298 | Cys241 |

Two of these loops are the preferred sites into which the EPM peptide may be inserted: N₁ His289 (i.e. insertion in the region of residues 286 to 292, which may include effective replacement of one or more residues with the EMP peptide) and N₂ Asp166 (i.e. insertion in the region of residues 162-170, which may include effective replacement of one or more residues with the EMP peptide). These positions give the correct orientation required for binding to the two halves of the EPO receptor. As the insertion sites are on the N₁ and N₂ domains of the N lobe, they have the flexibility of the hinge between these two sub domains.

Due to the structural similarity between the N and C lobes, the equivalent insertion sites on the C lobe (C₁ 489-495, C₂ 623-628) may also be used to make the molecule multivalent. This is done using a variety of the potential insert sites indicated above either on just the N or C lobe or by a combination of sites on both lobes.

Structural Alignment

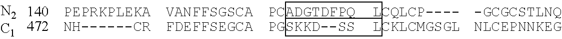

Amino Acid Alignment

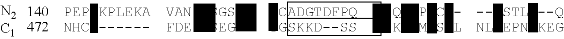

Structural Alignment

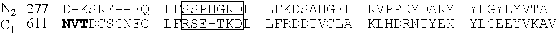

Amino Acid Alignment

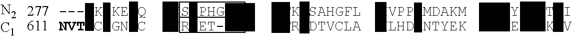

N₁ = SEQ ID NO: 6
N₂ = SEQ ID NO: 7
C₁ = SEQ ID NO: 8
C₂ = SEQ ID NO: 9

Steps for Producing the EPM Peptide/mTf Fusion Protein

In this Example, two EPM peptides are engineered into the transferrin scaffold using the encoding nucleic acids of the peptides and mTf.

```
1 ggtggtactt actcttgtca ttttggtcca ttgacttggg tttgtaagcc acaaggtggt
        g g t y s c h f g p l t w v c k p q g g
nucleic acid sequence: SEQ ID NO: 10;
amino acid sequence: SEQ ID NO: 4
C-C deletion (G substitution)
1 ggtggtactt actctggtca ttttggtcca ttgacttggg ttggtaagcc acaaggtggt
        g g t y s g h f g p l t w v g k p q g g
nucleic acid sequence: SEQ ID NO: 27;
amino acid sequence: SEQ ID NO: 28
```

Additional illustrative combinations include:

| | |
|---|---|
| g g t y s c h f g p l t w v c k p q g g | (SEQ ID NO: 4) |
| g g t y s g h f g p l t w v g k p q g g | (SEQ ID NO: 28) |
| g g t y s s h f g p l t w v s k p q g g | (SEQ ID NO: 34) |
| g g t y s x h f g p l t w v x k p q g g | (SEQ ID NO: 35) |
| t y s c h f g p l t w v c k p q | (residues 3-18 of SEQ ID NO: 4) |
| t y s g h f g p l t w v g k p q | (residues 3-18 of SEQ ID NO: 28) |
| t y s s h f g p l t w v s k p q | (residues 3-18 of SEQ ID NO: 34) |
| t y s x h f g p l t w v x k p q | (residues 3-18 of SEQ ID NO: 35) |
| c h f g p l t w v c | (residues 6-15 of SEQ ID NO: 4) |
| g h f g p l t w v g | (residues 6-15 of SEQ ID NO: 28) |
| s h f g p l t w v s | (residues 6-15 of SEQ ID NO: 34) |
| x h f g p l t w v x | (residues 6-15 of SEQ ID NO: 35) |
| h f g p l t w v | (residues 7-14 of SEQ ID NO: 4, 28, 34 and 35) |

where x is any amino acid other than c.

An EPM peptide is engineered into mTf between His289 and Gly290. The duplication inherent to the transferrin molecule, with the two lobes mirroring each other, makes it possible to engineer a second EPM peptide into the duplicate region of the C lobe, between Glu625 and Thr626.

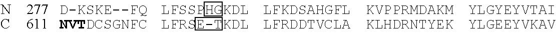

N domain: SEQ ID NO: 6
C domain: SEQ ID NO: 9

The N1 EPM graft at position 289 was inserted into mTf using overlapping primer sequences, P0141 and P0142, encoding the peptide and the adjoining mTf sequence.

Using these primers, along with primers 5′ and 3′ of the EcoRI and HpaI sites in mTf, P0141 with P0011 and P0141 with P0031, PCR products were generated. The products of these PCR reactions were then joined using the outer primers P0011 and P0031 in a further PCR reaction. The product was digested with EcoRI and HpaI and cloned into the mTf vector pREX0052 (see WO 04/020405, which is incorporated herein in its entirety) cut with EcoRI/HpaI to create pREX0387.

| (SEQ ID NO: 36): | |
|---|---|
| | EcoRI |
| | -+---- |
| 1 | tctcaaccag gcccaggaac attttggcaa agacaaatca aaagaattcc aactattcgg |
| | agagttggtc cgggtccttg taaaaccgtt tctgtttagt tttcttaagg ttgataagcc |
| | P0141 >> |
| | EPM graft >> |
| | <<............P0142..............< |
| 61 | tggtacttac tcttgtcatt ttggtccatt gacttgggtt tgtaagccac atgggaagga |
| | accatgaatg agaacagtaa aaccaggtaa ctgaacccaa acattcggtg tacccttcct |
| | >.............................P0141.............................> |
| | >.....................EPM graft....................>> |
| | g g t y s c h f g p l t w v c k p |
| | (residues 1-17 of SEQ ID NO: 4) |
| | <.......................P0142......................<< |
| 121 | cctgctgttt aaggactctg cccacgggtt tttaaaagtc ccccccagga tggatgccaa |
| | ggacgacaaa ttcctgagac gggtgcccaa aaattttcag ggggggtcct acctacggtt |
| | >.....P0141.....>> |
| 181 | gatgtacctg qgctatgagt atgtcactgc catccggaat ctacgggaag gcacatgccc |
| | ctacatggac ccgatactca tacagtgacg gtaggcctta gatgcccttc cgtgtacggg |
| 241 | agaagcccca acagatgaat gcaagcctgt gaagtggtgt gcgctgagcc accacgagag |
| | tcttcggggt tgtctactta cgttcggaca cttcaccaca cgcgactcgg tggtgctctc |
| | HpaI |
| | ---+-- |
| 301 | gctcaagtgt gatgagtgga gtgttaacag |
| | cgagttcaca ctactcacct cacaattgtc |

Primer Sequences

P0141

(SEQ ID NO: 37)

GGTGGTACTTACTCTTGTCATTTTGGTCCATTGACTTGGGTTTGTAAGCC
ACATGGGAAGGACCTGCTGTTTAAGGACT
P0142

(SEQ ID NO: 38)

TGGCTTACAAACCCAAGTCAATGGACCAAAATGACAAGAGTAAGTACCAC
CGAATAGTTGGAATTCTTTTGATTTGTCTT
P0011

(SEQ ID NO: 39)

TACACAGCTTACAGAGACTG
P0031

(SEQ ID NO: 40)

TACTGTGACTTACCTGAGCC

The N2 graft at position 166 was inserted using the method as described above.

The PCR product of primer sets P0143 and P0101 was joined to the product of P0144 and P0090 by a second round of PCR. This product was digested with XbaI and EcoRI and cloned into the mTf vector pREX0052 to create pREXO155.

| (SEQ ID NO: 41): | |
|---|---|
| | XbaI |
| | -+----- |
| 1 | aggtctctag agaaaagggt acctgataaa actgtgagat ggtgtgcagt gtcggagcat |
| | tccagagatc tcttttccca tggactattt tgacactcta ccacacgtca cagcctcgta |
| 61 | gaggccacta agtgccagag tttccgcgac catatgaaaa gcgtcattcc atccgatggt |
| | ctccggtgat tcacggtctc aaaggcgctg gtatactttt cgcagtaagg taggctacca |
| 121 | cccagtgttg cttgtgtgaa gaaagcctcc taccttgatt gcatcagggc cattgcggca |
| | gggtcacaac gaacacactt ctttcggagg atggaactaa cgtagtcccg gtaacgccgt |
| 181 | aacgaagcgg atgctgtgac actggatgca ggtttggtgt atgatgctta cctggctccc |
| | ttgcttcgcc tacgacactg tgacctacgt ccaaaccaca tactacgaat ggaccgaggg |
| 241 | aataacctga agcctgtggt ggcagagttc tatgggtcaa aagaggatcc acagactttc |
| | ttattggact tcggacacca ccgtctcaag atacccagtt ttctcctagg tgtctgaaag |
| 301 | tattatgctg ttgctgtggt gaagaaggat agtggcttcc agatgaacca gcttcgaggc |
| | ataatacgac aacgacacca cttcttccta tcaccgaagg tctacttggt cgaagctccg |
| 361 | aagaagtcct gccacacggg tctaggcagg tccgctgggt ggaacatccc cataggctta |
| | ttcttcagga cggtgtgccc agatccgtcc aggcgaccca ccttgtaggg gtatccgaat |
| 421 | ctttactgtg acttacctga gccacgtaaa cctcttgaga aagcagtggc caatttcttc |
| | gaaatgacac tgaatggact cggtgcattt ggagaactct ttcgtcaccg gttaaagaag |
| | P0144 >> |
| 481 | tcgggcagct gtgccccttg tgcggatgga acatattcat gtcacttcgg tcctttaaca |
| | agcccgtcga cacggggaac acgcctacct tgtataagta cagtgaagcc aggaaattgt |
| | >.............................P0144.............................> |
| | >>..........EPM graft..............> |
| | g t y s c h f g p l t |
| | <<.............P0143...............> |
| 541 | tgggtatgta aacctcaact gtgtcaactg tgtccagggt gtggctgctc cacccttaac |
| | acccatacat ttggagttga cacagttgac acaggtccca caccgacgag gtgggaattg |
| | >......P0144.....>> |
| | >..EPM graft.....>> |
| | w v c k p q (residues 2-18 of SEQ ID NO: 4) |
| | <.....................P0143......................<< |
| 601 | caatacttcg gctactcggg agccttcaag tgtctgaagg atggtgctgg ggatgtggcc |
| | gttatgaagc cgatgagccc tcggaagttc acagacttcc taccacgacc cctacaccgg |
| 661 | tttgtcaagc actcgactat atttgagaac ttggcaaaca aggctgacag ggaccagtat |
| | aaacagttcg tgagctgata taaactcttg aaccgtttgt tccgactgtc cctggtcata |
| 721 | gagctgcttt gcctggacaa cacccggaag ccggtagatg aatacaagga ctgccacttg |
| | ctcgacgaaa cggacctgtt gtgggccttc ggccatctac ttatgttcct gacggtgaac |
| 781 | gcccaggtcc cttctcatac cgtcgtggcc cgaagtatgg gcggcaagga ggacttgatc |
| | cgggtccagg gaagagtatg gcagcaccgg gcttcatacc cgccgttcct cctgaactag |
| | EcoRI |
| | -+---- |
| 841 | tgggagcttc tcaaccaggc ccaggaacat tttggcaaag acaaatcaaa agaattccaa |
| | accctcgaag agttggtccg ggtccttgta aaaccgttto tgtttagttt tcttaaggtt |
| 901 | ctat |
| | gata |

Primer Sequences

P0143

(SEQ ID NO: 42)

GGAACATATTCATGTCACTTCGGTCCTTTAACATGGGTATGTAAACCTCA
ACTGTGTCAACTGTGTCCAGGGTGTGGCTGC
P0144

(SEQ ID NO: 43)

TTGAGGTTTACATACCCATGTTAAAGGACCGAAGTGACATGAATATGTTC
CATCCGCACAAGGGGCACAGCTGCCCGAGA
P0101

(SEQ ID NO: 44)

CATGTCTAAGCTTATTATTCATCTGTTGGGGCTTCTGGGC
P0090

(SEQ ID NO: 45)

CAAGCTAAACCTAATTCTAAC

To create a plasmid with both EPM grafts, pREX0155 was digested with XbaI and EcoRI and the resulting fragment was cloned into pREX0387 previously digested with XbaI/EcoRI to create plasmid pREX0341.

In order to mutate the cysteine residues in the EPM loop at position 289 to glycine residues, mutagenic primers were created with the glycine codon GGT substituted for the cysteine codon. The product of mutagenic primer P0226 and a primer 3′ of the HpaI site (P0011) was joined to the product of primer P0227 with a primer 5′ of the EcoRI site (P0031) as described above. This product was restriction digested with EcoRI/HpaI and ligated into HpaI/EcoRI digested pREX0052 to make the plasmid pREX0607.

| (SEQ ID NO: 46): | |
|---|---|
| | EcoRI |
| | -+----- |
| 1 | tcaaaagaat tccaactatt cggtggtact tactctggtc attttggtcc attgacttgg |
| | agttttctta aggttgataa gccaccatga atgagaccag taaaaccagg taactgaacc |
| | >>................EPM289.................> |
| | g g t y s g h f g p l t w |
| | >>..............P0226................> |
| | <<..............P0227................< |
| 61 | gttggtaagc cacatgggaa ggacctgctg tttaaggact ctgcccacgg gtttttaaaa |
| | caaccattcg gtgtaccctt cctggacgac aaattcctga gacgggtgcc caaaaatttt |
| | >..EPM289..>> |
| | v g k p (residues 1-17 of SEQ ID NO: 28) |
| | >.....P0226....>> |
| | <.....P0227....<< |
| 121 | gtccccccca ggatggatgc caagatgtac ctgggctatg agtatgtcac tgccatccgg |
| | cagggggggt cctacctacg gttctacatg gacccgatac tcatacagtg acggtaggcc |
| 181 | aatctacggg aaggcacatg cccagaagcc ccaacagatg aatgcaagcc tgtgaagtgg |
| | ttagatgccc ttccgtgtac gggtcttcgg ggttgtctac ttacgttcgg acacttcacc |
| | HpaI |
| | ---+--- |
| 241 | tgtgcgctga gccaccacga gaggctcaag tgtgatgagt ggagtgttaa cagt |
| | acacgcgact cggtggtgct ctccgagttc acactactca cctcacaatt gtca |

In order to mutate the cysteine residues in the EPM loop at position 166 to glycine residues, mutagenic primers were created with the glycine codon GGT substituted for the cysteine codon. The product of mutagenic primer P0228 and a primer 3′ of the EcoRI site (P0101) was joined to the product of primer P0229 with a primer 5′ of the XbaI site (P0090). This product was restriction digested XbaI and EcoRI and ligated with EcoRI/XbaI restriction digested pREX0052 to make the plasmid pREX0242.

| (SEQ ID NO: 47): | |
|---|---|
| | XbaI |
| | -+----- |
| 1 | tctaggtctc tagagaaaag ggtacctgat aaaactgtga gatggtgtgc agtgtcggag |
| | agatccagag atctcttttc ccatggacta ttttgacact ctaccacacg tcacagcctc |
| 61 | catgaggcca ctaagtgcca gagtttccgc gaccatatga aaagcgtcat tccatccgat |
| | gtactccggt gattcacggt ctcaaaggcg ctggtatact tttcgcagta aggtaggcta |
| 121 | ggtcccagtg ttgcttgtgt gaagaaagcc tcctaccttg attgcatcag ggccattgcg |
| | ccagggtcac aacgaacaca cttctttcgg aggatggaac taacgtagtc ccggtaacgc |
| 181 | gcaaacgaag cggatgctgt gacactggat gcaggtttgg tgtatgatgc ttacctggct |
| | cgtttgcttc gcctacgaca ctgtgaccta cgtccaaacc acatactacg aatggaccga |
| 241 | cccaataacc tgaagcctgt ggtggcagag ttctatgggt caaaagagga tccacagact |
| | gggttattgg acttcggaca ccaccgtctc aagataccca gttttctcct aggtgtctga |
| 301 | ttctattatg ctgttgctgt ggtgaagaag gatagtggct tccagatgaa ccagcttcga |
| | aagataatac gacaacgaca ccacttcttc ctatcaccga aggtctactt ggtcgaagct |
| 361 | ggcaagaagt cctgccacac gggtctaggc aggtccgctg ggtggaacat ccccataggc |
| | ccgttcttca ggacggtgtg cccagatccg tccaggcgac ccaccttgta ggggtatccg |
| 421 | ttactttact gtgacttacc tgagccacgt aaacctcttg agaaagcagt ggccaatttc |
| | aatgaaatga cactgaatgg actcggtgca tttggagaac tctttcgtca ccggttaaag |
| 481 | ttctcgggca gctgtgcccc ttgtgcggat ggaacatatt caggtcactt cggtccttta |
| | aagagcccgt cgacacgggg aacacgccta ccttgtataa gtccagtgaa gccaggaaat |
| | >>...........EPM166............> |
| | g t y s g h f g p l |
| | >>.........P0228...........> |
| | <<.........P0229...........< |
| 541 | acatgggtag gtaaacctca actgtgtcaa ctgtgtccag ggtgtggctg ctccaccctt |
| | tgtacccatc catttggagt tgacacagtt gacacaggtc ccacaccgac gaggtgggaa |
| | >.......EPM166.......>> |
| | t w v g k p q (residues 2-18 of SEQ ID NO: 28) |
| | >.........P0228.........>> |
| | <.........P0229.........<< |
| 601 | aaccaatact tcggctactc gggagccttc aagtgtctga aggatggtgc tggggatgtg |
| | ttggttatga agccgatgag ccctcggaag ttcacagact tcctaccacg acccctacac |
| 661 | gcctttgtca agcactcgac tatatttgag aacttggcaa acaaggctga cagggaccag |
| | cggaaacagt tcgtgagctg atataaactc ttgaaccgtt tgttccgact gtccctggtc |
| 721 | tatgagctgc tttgcctgga caacacccgg aagccggtag atgaatacaa ggactgccac |
| | atactcgacg aaacggacct gttgtgggcc ttcggccatc tacttatgtt cctgacggtg |
| 781 | ttggcccagg tcccLtctca taccgtcgtg gcccgaagta tgggcggcaa ggaggacttg |
| | aaccgggtcc agggaagagt atggcagcac cgggcttcat acccgccgtt cctcctgaac |
| | EcoRI |
| | -+---- |
| 841 | atctgggagc ttctcaacca ggcccaggaa cattttggca aagacaaatc aaaagaattc |
| | tagaccctcg aagagttggt ccgggtcctt gtaaaaccgt ttctgtttag ttttcttaag |
| 901 | caa |
| | gtt |

To create a plasmid with both EPM graft loops with mutated cysteine residues, pREX0607 was digested with EcoRI/HpaI and the fragment ligated into pREX0242 digested with EcoRI/HpaI to create pREX0317.

To create the final expression vectors for transformation in to a yeast host cell, pREX0341 and pREX0317 were digested with NotI and ligated into NotI digested pSAC35 (Sleep et al., 1991, Bio/Technology 9,183-187 and EP 431 880 B) to create pREX0413 and pREX0318 respectively.

The resultant plasmid is transformed into yeast for protein expression.

Alternative points for insertion of the EPM peptide or any other peptide(s) are the two glycosylation sites on the C lobe of Transferrin at N413 and N611. The advantage of these sites is that once insertion is achieved, glycosylation is prevented through disruption of the N-X-S/T sequence.

### Example 2

### Preparation of Therapeutic Transferrin Fusion Proteins with Increased Iron Affinity

Therapeutic fusion proteins with increased iron affinity may be prepared. As an example, preparing modified transferrin fusion proteins with increased iron binding ability, the procedure in Example 1 above may be carried out with the following modification. These fusion proteins may be used to facilitate uptake and transfer of the fusion protein across the gastrointestinal epithelium.

A cloning vector which contains the mTf sequence is cut with a restriction enzyme, or a pair of restriction enzymes, to remove a portion of the mTf gene. Using techniques standard in the art, this fragment is then subjected to site-directed mutagenesis using primers that introduce a mutation at a position corresponding to nucleotide 723 of SEQ ID NO: 1, converting the codon AAG (Lys) to CAG (Gln) or GAG (Glu). Similarly, primers are used that introduce mutations at positions corresponding to nucleotides 726 and 728 of SEQ ID NO: 1, converting the codon CAC (His) to CAG (Gln) or GAG (Glu). Primers may also be used that introduce mutations in the adjacent codons, resulting in the substitution of the encoded amino acids. These nucleotide positions correspond to amino acids 225 and 226 of the protein encoded with the leader sequence and to amino acids 206 and 207 of the mature protein. The mutated fragment is then amplified by PCR and religated into the cloning vector. This vector containing the mutation or mutations is used in a subsequent step for introduction of a DNA molecule coding for the EPM peptide. The mTf fusion protein sequence may be introduced into yeast expression vectors and transformed into Saccharomyces or other yeasts for protein production.

Other amino acids may also be mutated to obtain therapeutic Tf fusion proteins with increased iron affinity.

### Example 3

### Preparation Of EMP1 Fusion Proteins with Improved Productivity

The present Example provides a method of generating EMP1 fusion proteins with improved productivity through changing the hydrophobic nature of the EMP1 peptide.

A hydrophobicity plot (Kyte-Doolittle) of the EMP-1 peptide inserted into mTf shows a stretch of hydrophobicity (score>zero) at the core of the EMP1 peptide. This hydrophobic core is composed of the Gly-Pro-Leu-Thr-Trp (residues 9-13 of SEQ ID NO: 4, 28, 34 and 35) residues (in bold below).

Molecule: pREX0381
Region: 1774 to 1824

| | |
|---|---|
| (residues 1-17 of SEQ ID NO: 28) | Kyte-Doolittle |
| Res Amino-Acid | (+4 to −4) |

| | | | |
|---|---|---|---|
| 1 | G | Gly | |
| 2 | G | Gly | |
| 3 | T | Thr | −0.720 |
| 4 | Y | Tyr | −0.720 |
| 5 | S | Ser | −1.280 |
| 6 | G | Gly | −0.580 |
| 7 | H | His | −0.400 |
| 8 | F | Phe | −0.560 |
| 9 | G | Gly | 0.280 |
| 10 | P | Pro | 0.780 |
| 11 | L | Leu | 0.040 |
| 12 | T | Thr | 0.960 |
| 13 | W | Trp | 1.200 |
| 14 | V | Val | −0.340 |
| 15 | G | Gly | −0.520 |
| 16 | K | Lys | |
| 17 | P | Pro | |

The introduction of the EMP-1 peptide on to the surface of mTf results in a hydrophobic projection from that surface. Changing the hydrophobic nature of the peptide insert, without substantially reducing its ability to bind its target, may result in improved productivity.

The residues at positions 9, 10, 12 and 13 in the hydrophobic core, highlighted in bold above, are included in the motif necessary for receptor binding. The only residue in the hydrophobic core not involved is the leucine at position the 11. Substitution of this residues can have the effect of reducing the calculated hydrophobicity of the hydrophobic core making it more hydrophilic (score<zero). An example of substituting the leucine residue with a glutamic acid residue is given below.

| | |
|---|---|
| (SEQ ID NO: 48) | Kyte-Doolittle |
| Res Amino-Acid | (+4 to −4) |

| | | | |
|---|---|---|---|
| 1 | G | Gly | |
| 2 | G | Gly | |
| 3 | T | Thr | −0.720 |
| 4 | Y | Tyr | −0.720 |
| 5 | S | Ser | −1.280 |
| 6 | G | Gly | −0.580 |
| 7 | H | His | −0.400 |
| 8 | F | Phe | −0.560 |
| 9 | G | Gly | −1.180 |
| 10 | P | Pro | −0.680 |
| 11 | E | Glu | −1.420 |
| 12 | T | Thr | −0.500 |
| 13 | W | Trp | −0.260 |
| 14 | V | Val | −0.340 |
| 15 | G | Gly | −0.520 |
| 16 | K | Lys | |
| 17 | P | Pro | |

The valine residue at position 14, also not involved in receptor binding, bordering the hydrophobic core is in close enough proximity that its substitution can also influence the hydrophobic core. An example is substituting the valine for glutamic acid is given below.

| | |
|---|---|
| (SEQ ID NO: 49) | Kyte-Doolittle |
| Res Amino-Acid | (+4 to −4) |

| | | | |
|---|---|---|---|
| 1 | G | Gly | |
| 2 | G | Gly | |
| 3 | T | Thr | −0.720 |
| 4 | Y | Tyr | −0.720 |
| 5 | S | Ser | −1.280 |
| 6 | G | Gly | −0.580 |
| 7 | H | His | −0.400 |
| 8 | F | Phe | −0.560 |
| 9 | G | Gly | 0.280 |
| 10 | P | Pro | 0.780 |
| 11 | L | Leu | 0.040 |
| 12 | T | Thr | −0.580 |
| 13 | W | Trp | −0.340 |
| 14 | E | Glu | −1.880 |
| 15 | G | Gly | −2.060 |
| 16 | K | Lys | |
| 17 | P | Pro | |

Substitution of both the leucine and the valine residues has a combined effect in decreasing hydrophobicity. An example of substituting both the leucine and the valine for glutamic acid is given below.

Molecule: pREX0593
Region: 1774 to 1824

| | |
|---|---|
| (SEQ ID NO: 50) | Kyte-Doolittle |
| Res Amino-Acid | (+4 to −4) |

| | | | |
|---|---|---|---|
| 1 | G | Gly | |
| 2 | G | Gly | |
| 3 | T | Thr | −0.720 |
| 4 | Y | Tyr | −0.720 |
| 5 | S | Ser | −1.280 |
| 6 | G | Gly | −0.580 |
| 7 | H | His | −0.400 |
| 8 | F | Phe | −0.560 |
| 9 | G | Gly | −1.180 |
| 10 | P | Pro | −0.680 |
| 11 | E | Glu | −1.420 |
| 12 | T | Thr | −2.040 |
| 13 | W | Trp | −1.800 |
| 14 | E | Glu | −1.880 |
| 15 | G | Gly | −2.060 |
| 16 | K | Lys | |
| 17 | P | Pro | |

An additional example of substituting the leucine for a threonine and the valine for an aspartic acid residue is given below.

Molecule: pREX0594
Region: 1774 to 1824

| | |
|---|---|
| (SEQ ID NO: 51) | Kyte-Doolittle |
| Res Amino-Acid | (+4 to −4) |

| | | | |
|---|---|---|---|
| 1 | G | Gly | |
| 2 | G | Gly | |
| 3 | T | Thr | −0.720 |
| 4 | Y | Tyr | −0.720 |
| 5 | S | Ser | −1.280 |
| 6 | G | Gly | −0.580 |
| 7 | H | His | −0.400 |
| 8 | F | Phe | −0.560 |
| 9 | G | Gly | −0.620 |
| 10 | P | Pro | −0.120 |
| 11 | T | Thr | −0.860 |
| 12 | T | Thr | −1.480 |
| 13 | W | Trp | −1.240 |
| 14 | D | Asp | −1.880 |
| 15 | G | Gly | −2.060 |
| 16 | K | Lys | |
| 17 | P | Pro | |

Substitution of the residues outlined above was achieved by essentially the same process of mutagenesis that was used to substitute the cycstine residues in EMP for glycine to remove the disulphide bond as described previously.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

What is claimed:

1. An EPM peptide comprising:
(a) a first modification of at least one cysteine residue of EMP-1 that substantially reduces disulfide bond formation; and
(b) a second modification such that the peptide exhibits EMP-1 activity.

2. The EPM peptide of claim 1, wherein the first modification comprises the deletion of at least one cysteine residue from an EMP-1 peptide sequence.

3. The EPM peptide of claim 1, wherein the first modification comprises a substitution of at least one cysteine residue from an EMP-1 peptide sequence.

4. The EPM peptide of claim 1, wherein the second modification comprises the addition of a linker group that is covalently bonded to the C-terminal amino acid of EMP-1.

5. The EPM peptide of claim 1, wherein the second modification comprises the addition of a linker group that is covalently bonded to the N-terminal amino acid of EMP-1.

6. The EPM peptide of claim 1, wherein the second modification comprises the addition of a first linker group that is covalently bonded to the N-terminal amino acid and a second linker group that is convalently bonded to the C-terminal amino acid of EMP-1.

7. The EPM peptide of claim 1, wherein the first modification comprises the deletion of at least one cysteine residue and the second modification comprises the addition of at least one linker group that is covalently bonded to EMP-1.

8. The EPM peptide of claim 8, wherein the first modification comprises the deletion of two cysteine residues.

9. The EPM peptide of claim 3, wherein the amino acid substituting the at least one cysteine residue allows circularization of the peptide.

10. The EPM peptide of claim 10, wherein the amino acid is aspartic acid.

11. The EPM peptide of claim 1, wherein the first modification reduces binding of the peptide to the erythropoietin receptor in the absence of the second modification.

12. The EPM peptide of claim 11, wherein the second modification restores detectable binding of the peptide to the erythyropoietin receptor.

13. The EPM peptide of claim 1, wherein the activity is binding to the erythropoietin receptor.

14. The EPM peptide of claim 1, wherein the activity is activation of the erythropoietin receptor.

15. A fusion protein comprising an EPM peptide of claim 1 fused to a second peptide or protein.

16. The fusion protein of claim 15, wherein the EPM peptide exhibits increased serum stability or in vivo circulatory half-life compared to EMP-1.

17. The fusion protein of claim 15, wherein the second peptide or protein is transferrin, melanotransferrin, lactoferrin, maltose binding protein, green fluorescent protein, an immunoglobulin, an Fc fragment of an immunoglobulin, or glutathione S-transferase.

18. The fusion protein of claim 17, wherein the second peptide or protein is transferrin.

19. The fusion protein of claim 18, wherein the transferrin protein exhibits reduced glycosylation.

20. The fusion protein of claim 18, wherein the transferrin protein exhibits reduced metal binding.

21. The fusion protein of claim 18, wherein the transferrin protein exhibits reduced receptor binding or the protein does not bind a transferrin receptor.

22. The fusion protein of claim 18, wherein an EPM peptide is fused to the C-Terminal end of transferrin.

23. The fusion protein of claim 18, wherein an EPM peptide is fused to the N-Terminal end of transferrin.

24. The fusion protein of claim 18, wherein an EPM peptide is fused to the N-Terminal end and the C-terminal end of transferrin.

25. The fusion protein of claim 18, wherein an EPM peptide is inserted into at least one loop of transferrin.

26. The fusion protein of claim 18, wherein an EPM peptide is inserted into at least two loops of transferrin.

27. The fusion protein of claim 18, wherein the transferrin protein has reduced affinity for a transferrin receptor.

28. The fusion protein of claim 18, wherein an EPM peptide is fused to the second peptide or protein via a linker group.

29. The fusion protein of claim 28, wherein the linker group is a peptide chain.

30. The fusion protein of claim 29, wherein the linker group is a polyglycine stretch.

31. A nucleic acid molecule encoding a peptide of claim 1.

32. A nucleic acid molecule encoding a protein of claim 18.

33. A vector comprising a nucleic acid molecule of claim 31 or 32.

34. A host cell comprising a vector of claim 33.

35. A host cell comprising a nucleic acid molecule of claim 31.

36. A method of expressing a Tf fusion protein comprising culturing a host cell of claim 35 under conditions, which express an encoded fusion protein.

37. A method of expressing a Tf fusion protein comprising culturing a host cell of claim 35 under conditions which express the encoded fusion protein.

38. A host cell of claim 34, wherein the cell is prokaryotic or eukaryotic.

39. A host cell of claim 35, wherein the cell is prokaryotic or eukaryotic.

40. A host cell of claim 34, wherein the cell is a yeast cell.

41. A host cell of claim 35, wherein the cell is a yeast cell.

42. A transgenic animal comprising a nucleic acid molecule of 31.

43. A method of producing a fusion protein comprising isolating a fusion protein from a transgenic animal of claim 42.

44. A method of claim 43, wherein the fusion protein is isolated from a biological fluid from the transgenic animal.

45. A pharmaceutical composition comprising the EPM peptide of claim 1 and a pharmaceutically acceptable carrier.

46. A pharmaceutical composition comprising the fusion protein of claim 18 and a pharmaceutically acceptable carrier.

47. A method of treating or preventing a disease or disease symptom in a patient, which comprises administering to a patient in need thereof a therapeutically or prophylactically effective amount of the EPM peptide of claim 1.

48. A method of treating or preventing a disease or disease symptom in a patient, which comprises administering to a patient in need thereof a therapeutically or prophylactically effective amount of a fusion protein of claim 18.

49. The method of claim 47 or 48, wherein the patient is suffering from multiple sclerosis, brain tumor, skin cancer, hepatitis B, or hepatitis C.

50. The method of claim 47 or 48, wherein the subject is suffering from low or defective red blood cell production as compared to a healthy subject.

51. The method of claim 50, wherein the low or defective red blood cell production is associated with anemia, β-thalassemia, pregnancy or menstrual disorders, rheumatoid arthritis, AIDS, and cancer.

52. The method of claim 47 or 48, wherein the patient is suffering from viral disease or infections, cancer, a metabolic diseases, obesity, autoimmune diseases, inflammatory diseases, allergy, graft-vs.-host disease, systemic microbial infection, cardiovascular disease, psychosis, genetic diseases, neurodegenerative diseases, disorders of hematopoietic cells, diseases of the endocrine system or reproductive systems, gastrointestinal diseases, diabetes, multiple sclerosis, asthma, HCV or HIV infections, hypertension, hypercholesterolemia, arterial scherosis, arthritis, or Alzheimer's disease.

53. An EPM peptide of any one of claims 4, 5, or 28 wherein the linker is (Pro-Glu-Ala-Pro-Thr-Asp)_{y} (SEQ ID NO: 32) and wherein y is 1, 2, 3, 4, 5, 6, 7, or 8.

54. An EPM peptide comprising:
(a) a first modification of EMP-1 comprising a replacement of a hydrophobic residue with a less hydrophobic residue; and
(b) a second modification such that the peptide exhibits EMP-1 activity.

55. An EPM peptide of claim 54, wherein the hydrophobic residue is Leu11 or Val14.

56. An EPM peptide of claim 55, wherein Leu11 or Val14 is replaced with a hydrophilic residue selected from the group consisting of Glu, Asp, Lys, Arg, His, Asn, Gln, and Ser.

57. An EPM peptide of claim 56, wherein Leu11 is replaced with Glu or Thr.

58. An EPM peptide of claim 56, wherein Val14 is replaced with Glu or Asp.

59. An EPM peptide of claim 1 in wherein the amino acid sequence is reversed with respect to that in EMP-1.

60. A peptide linker comprising the sequence (Pro-Glu-Ala-Pro-Thr-Asp)_{y} (SEQ ID NO: 32) and wherein y is 1, 2, 3, 4, 5, 6, 7, or 8.


SEQUENCE LISTING (51 sequences)

SEQ ID NO: 1 - DNA, 2318 bp, Homo sapiens
  CDS (51)..(2147): GenBank Acc. No. NM_001063, transferrin gene and protein
  sig_peptide (51)..(107)
gcacagaagcgagtccgactgtgctcgctgctcagcgccgcacccggaagatgaggctcgccgtgggagccctgctggtctgcgccgtcctggggctgtgtctggctgtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaaaatctcagaggtagggctgccaccaaggtgaagatgggaacgcagatgatccatgagtttgccctggtttcactggcccaagtggtttgtgctaaccacgtctgtcttcacagctctgtgttgccatgtgtgctgaacaaaaaataaaaattattattgattttatatttc

SEQ ID NO: 2 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 3 - protein, 679 aa, Homo sapiens
  MISC_FEATURE: Mature Transferrin Protein
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 4 - protein, 20 aa, Artificial Sequence
  EMP1 peptide
GGTYSCHFGPLTWVCKPQGG

SEQ ID NO: 5 - protein, 12 aa, Artificial Sequence
  Neutrophil lactoferrin splice variant
EDCIALKGEADA

SEQ ID NO: 6 - protein, 47 aa, Homo sapiens
  misc_feature: N1 subdomain of transferrin
DKSKEFQLFSSPHGKDLLFKDSAHGFLKVPPRMDAKMYLGYEYVTAI

SEQ ID NO: 7 - protein, 45 aa, Homo sapiens
  misc_feature: N2 subdomain of Transferrin
PEPRKPLEKAVANFFSGSCAPCADGTDFPQLCQLCPGCGCSTLNQ

SEQ ID NO: 8 - protein, 42 aa, Homo sapiens
  misc_feature: C1 subdomain of transferrin
NHCRFDEFFSEGCAPGSKKDSSLCKLCMGSGLNLCEPNNKEG

SEQ ID NO: 9 - protein, 49 aa, Homo sapiens
  misc_feature: C2 subdomain of transferrin
NVTDCSGNFCLFRSETKDLLFRDDTVCLAKLHDRNTYEKYLGEEYVKAV

SEQ ID NO: 10 - DNA, 60 bp, Artificial Sequence
  synthetic oligonucleotide encoding peptide with EPO activity
ggtggtacttactcttgtcattttggtccattgacttgggtttgtaagccacaaggtggt

SEQ ID NO: 11 - protein, 20 aa, Artificial Sequence
  EMP1 peptide
  MISC_FEATURE (1)..(20): Xaa = Gly, Ser or any amino acid but Cys
GGTYSXHFGPLTWVXKPQGG

SEQ ID NO: 12 - DNA, 140 bp, Artificial Sequence
  His289-Gly290 insert PCR product
agacaaatcaaaagaatttcaactattcagctctcctcatggtggtacttactcttgtcattttggtccattgacttgggtttgtaagccacaaggtggtgggaaggacctgctgtttaaggactctgcccacgggtttt

SEQ ID NO: 13 - DNA, 210 bp, Artificial Sequence
  Glu625-Thr626 insert PCR product
cctatttggaagcaacgtaactgactgctcgggcaacttttgtttgttccggtcggaaggtggtacttactcttgtcattttggtccattgacttgggtttgtaagccacaaggtggtaccaaggaccttctgttcagagatgacacagtatgtttggccaaacttcatgacagaaacacatatgaaaaatacttaggagaagaatatgt

SEQ ID NO: 14 - protein, 10 aa, Artificial Sequence
  EPO domain
CRIGPITWVC

SEQ ID NO: 15 - protein, 676 aa, Oryctolagus cuniculus
VTEKTVRWCAVNDHEASKCANFRDSMKKVLPEDGPRIICVKKASYLDCIKAIAAHEADAVTLDAGLVHEAGLTPNNLKPVVAEFYGSKENPKTFYYAVALVKKGSNFQLNELQGKKSCHTGLGRSAGWNIPIGLLYCDLPEPRKPLEKAVASFFSGSCVPCADGADFPQLCQLCPGCGCSSVQPYFGYSGAFKCLKDGLGDVAFVKQETIFENLPSKDERDQYELLCLDNTRKPVDEYEQCHLARVPSHAVVARSVDGKEDLIWELLNQAQEHFGKDKSGDFQLFSSPHGKNLLFKDSAYGFFKVPPRMDANLYLGYEYVTAVRNLREGICPDPLQDECKAVKWCALSHHERLKCDEWSVTSGGLIECESAETPEDCIAKIMNGEADAMSLDGGYVYIAGQCGLVPVLAENYESTDCKKAPEEGYLSVAVVKKSNPDINWNNLEGKKSCHTAVDRTAGWNIPMGLLYNRINHCRFDEFFRQGCAPGSQKNSSLCELCIGPSVCAPNNREGYYGYTGAFRCLVEKGDVAFVKSQTVLQNTGGRNSEPWAKDLKEEDFELLCLDGTRKPVSEAHNCHLAKAPNHAVVSRKDKAACVKQKLLDLQVEYGNTVADCSSKFCMFHSKTKDLLFRDDTKCLVDLRGKNTYEKYLGADYIKAVSNLRKCSTSRLLEACTFHKH

SEQ ID NO: 16 - protein, 676 aa, Rattus norvegicus
VPDKTVKWCAVSEHENTKCISFRDHMKTVLPADGPRLPCVKKTSYQDCIKAISGGEADAITLDGGWVYDAGLTPNNLKPVAAEFYGSLEHRQTHYLAVAVVKKGTDFQLNQLQGKKSCHTGLGRSAGWIIPIGLLFCNLPEPRKPLEKAVASFFSGSCVPCADPVAFPQLCQLCPGCGCSPTQPFFGYVGAFKCLRDGGGDVAFVKHTTIFEVLPQKADRDQYELLCLDNTRKPVDQYEDCYLARIPSHAVVARNGDGKEDLIWEILKVAQEHFGKGKSKDFQLFGSPLGKDLLFKDSRFGLLRAPKDGLQAVPRPQLCHCHSKSAGSCPDAIDSAPVKWCALSHQERAKCDEWSVTGNGQIECESAESTEDCIDKIVNGEADAMSLDGGHAYIAGQCGLVPVMAENYDISSCTNPQSDVFPKGYYAVAVVKASDSSINWNNLKGKKSCHTGVDRTAGWNIPMGLLFSRINHCKFDEFFSQGCAPGYKKNSTLCDLCIGPAKCAPNNREGYNGYTGAFQCLVEKGDVAFVKHQTVLENTNGKNTAAWAKDLKQEDFQLLCPDGTKKPVTEFATCHLAQAPNHVVVSRKEKAARVSTVLTAQKDLFWKGDKDCTGNFCLFRSSTKDLLFRDDTKCLTKLPEGTTYEEYLGAEYLQAVGNIRKCSTSRLLEACTFHKS

SEQ ID NO: 17 - protein, 677 aa, Mus musculus
VPDKTVKWCAVSEHENTKCISFRDHMKTVLPPDGPRLACVKKTSYPDCIKAISASEADAMTLDGGWVYDAGLTPNNLKPVAAEFYGSVEHPQTYYYAVAVVKKGTDFQLNQLEGKKSCHTGLGRSAGWVIPIGLLFCKLSEPRSPLEKAVSSFFSGSCVPCADPVAFPKLCQLCPGCGCSSTQPFFGYVGAFKCLKDGGGDVAFVKHTTIFEVLPEKADRDQYELLCLDNTRKPVDQYEDCYLARIPSHAVVARKNNGKEDLIWEILKVAQEHFGKGKSKDFQLFSSPLGKDLLFKDSAFGLLRVPPRMDYRLYLGHNYVTAIRNQQEGVCPEGSIDNSPVKWCALSHLERTKCDEWSIISEGKIECESAETTEDCIEKIVNGEADAMTLDGGHAYIAGQCGLVPVMAEYYESSNCAIPSQQGIFPKGYYAVAVVKASDTSITWNNLKGKKSCHTGVDRTAGWNIPMGMLYNRINHCKFDEFFSQGCAPGYEKNSTLCDLCIGPLKCAPNNKEEYNGYTGAFRCLVEKGDVAFVKHQTVLDNTEGKNPAEWAKNLKQEDFELLCPDGTRKPVKDFASCHLAQAPNHVVVSRKEKAARVKAVLTSQETLFGGSDCTGNFCLFKSTTKDLLFRDDTKCFVKLPEGTTPEKYLGAEYMQSVGNMRKCSTSRLLEACTFHK

SEQ ID NO: 18 - protein, 688 aa, Equus caballus
AEQTVRWCTVSNHEVSKCASFRDSMKSIVPAPPLVACVKRTSYLECIKAIADNEADAVTLDAGLVFEAGLSPYNLKPVVAEFYGSKTEPQTHYYAVAVVKKNSNFQLNQLQGKKSCHTGLGRSAGWNIPIGLLYWQLPEPRESLQKAVSNFFAGSCVPCADRTAVPNLCQLCVGKGTDKCACSNHEPYFGYSGAFKCLADGAGDVAFVKHSTVLENLPQEADRDEYQLLCRDNTRKSVDEYKDCYLASIPSHAVVARSVDGKEDLIWGLLNQAQEHFGTEKSKDFHLFSSPHGKDLLFKDSALGFLRIPPAMDTWLYLGYEYVTAIRNLREDIRPEVPKDECKKVKWCAIGHHEKVKCDEWSVNSGGNIECESAQSTEDCIAKIVKGEADAMSLDGGFIYIAGKCGLVPVLAENYETRSGSACVDTPEEGYHAVAVVKSSSDPDLTWNSLKGKKSCHTGVDRTAGWNIPMGLLYSEIKHCEFDKFFREGCAPGYRRNSTLCNLCIGSASGPGRECEPNNHERYYGYTGAFRCLVEKGDVAFVKHQTVEQNTDGRNPDDWAKDLKSENFKLLCPDGTRKSVTEFKSCYLARAPNHAVVSRKEKAACVCQELHNQQASYGKNGSHCPDKFCLFQSATKDLLFRDDTQCLANLQPTTTYKTYLGEKYLTAVANLRQCSTSRLLEACTFHRV

SEQ ID NO: 19 - protein, 685 aa, Bos taurus
DPERTVRWCTISTHEANKCASFRENVLRILESGPFVSCVKKTSHMDCIKAISNNEADAVTLDGGLVYEAGLKPNNLKPVVAEFHGTKDNPQTHYYAVAVVKKDTDFKLNELRGKKSCHTGLGRSAGWNIPMAKLYKELPDPQESIQRAAANFFSASCVPCADQSSFPKLCQLCAGKGTDKCACSNHEPYFGYSGAFKCLMEGAGDVAFVKHSTVFDNLPNPEDRKNYELLCGDNTRKSVDDYQECYLAMVPSHAVVARTVGGKEDVIWELLNHAQEHFGKDKPDNFQLFQSPHGKDLLFKDSADGFLKIPSKMDFELYLGYEYVTALQNLRESKPPDSSKDECMVKWCAIGHQERTKCDRWSGFSGGAIECETAENTEECIAKIMKGEADAMSLDGGYLYIAGKCGLVPVLAENYKTEGESCKNTPEKGYLAVAVVKTSDANINWNNLKDKKSCHTAVDRTAGWNIPMGLLYSKINNCKFDEFFSAGCAPGSPRNSSLCALCIGSEKGTGKECVPNSNERYYGYTGAFRCLVEKGDVAFVKDQTVIQNTDGNNNEAWAKNLKKENFEVLCKDGTRKPVTDAENCHLARGPNHAVVSRKDKATCVEKILNKQQDDFGKSVTDCTSNFCLFQSNSKDLLFRDDTKCLASIAKKTYDSYLGDDYVRAMTNLRQCSTSKLLEACTFHKP

SEQ ID NO: 20 - protein, 696 aa, Sus scrofa
  misc_feature (308)..(308): Xaa can be any naturally occurring amino acid
VAQKTVRWCTISNQEANKCSSFRENMSKAVKNGPLVSCVKKSSYLDCIKAIRDKEADAVTLDAGLVFEAGLAPYNLKPVVAEFYGQKDNPQTHYYAVAVVKKGSNFQWNQLQGKRSCHTGLGRSAGWIIPMGLLYDQLPEPRKPIEKAVASFFSSSCVPCADPVNFPKLCQQCAGKGAEKCACSNHEPYFGYAGAFNCLKEDAGDVAFVKHSTVLENLPDKADRDQYELLCRDNTRRPVDDYENCYLAQVPSHAVVARSVDGQEDSIWELLNQAQEHFGRDKSPDFQLFSSSHGKDLLFKDSANGFLXIPSKMDSSLYLGYQYVTALRNLREEISPDSSKNECKKVRWCAIGHEETQKCDAWSINSGGKIECVSAENTEDCIAKIVKGEADAMSLDGGYIYIAGKCGLVPVLAENYKTEGENCVNTPEKGYLAVAVVKKSSGPDLNWNNLKGKKSCHTAVDRTAGWNIPMGLLYNKINSCKFDQFFGEGCAPGSQRNSSLCALCIGSERAPGRECLANNHERYYGYTGAFRCLVEKGDVAFVKDQVVQQNTDGKNKDDWAKDLKQMDFELLCQNGAREPVDNAENCHLARAPNHAVVARDDKVTCVAEELLKQQAQFGRHVTDCSSSFCMFKSNTKDLLFRDDTQCLARVGKTTYESYLGADYITAVANLRKCSTSKLLEACTFHSAKNPRVETTT

SEQ ID NO: 21 - protein, 686 aa, Gallus gallus
APPKSVIRWCTISSPEEKKCNNLRDLTQQERISLTCVQKATYLDCIKAIANNEADAISLDGGQAFEAGLAPYKLKPIAAEVYEHTEGSTTSYYAVAVVKKGTEFTVNDLQGKTSCHTGLGRSAGWNIPIGTLLHRGAIEWEGIESGSVEQAVAKFFSASCVPGATIEQKLCRQCKGDPKTKCARNAPYSGYSGAFHCLKDGKGDVAFVKHTTVNENAPDQKDEYELLCLDGSRQPVDNYKTCNWARVAAHAVVARDDNKVEDIWSFLSKAQSDFGVDTKSDFHLFGPPGKKDPVLKDLLFKDSAIMLKRVPSLMDSQLYLGFEYYSAIQSMRKDQLTPSPRENRIQWCAVGKDEKSKCDRWSVVSNGDVECTVVDETKDCIIKIMKGEADAVALDGGLVYTAGVCGLVPVMAERYDDESQCSKTDERPASYFAVAVARKDSNVNWNNLKGKKSCHTAVGRTAGWVIPMGLIHNRTGTCNFDEYFSEGCAPGSPPNSRLCQLCQGSGGIPPEKCVASSHEKYFGYTGALRCLVEKGDVAFIQHSTVEENTGGKNKADWAKNLQMDDFELLCTDGRRANVMDYRECNLAEVPTHAVVVRPEKANKIRDLLERQEKRFGVNGSEKSKFMMFESQNKDLLFKDLTKCLFKVREGTTYKEFLGDKFYTVISSLKTCNPSDILQMCSFLEGK

SEQ ID NO: 22 - protein, 5 aa, Artificial Sequence
  unit of non-hindering or protease-resistant peptide linker
SGGGG

SEQ ID NO: 23 - protein, 15 aa, Artificial Sequence
  non-hindering or protease-resistant peptide linker
SGGGGSGGGGSGGGG

SEQ ID NO: 24 - protein, 17 aa, Artificial Sequence
  non-hindering or protease-resistant peptide linker
SGGGGSGGGGSGGGGSP

SEQ ID NO: 25 - protein, 5 aa, Artificial Sequence
  unit of non-hindering or protease-resistant peptide linker
SSSSG

SEQ ID NO: 26 - protein, 17 aa, Artificial Sequence
  non-hindering or protease-resistant peptide linker
SSSSGSSSSGSSSSGSP

SEQ ID NO: 27 - DNA, 60 bp, Artificial Sequence
  mutated EMP-1- C6G, C15G
  CDS (1)..(60)
ggtggtacttactctggtcattttggtccattgacttgggttggtaagccacaaggtggt

SEQ ID NO: 28 - protein, 20 aa, Artificial Sequence
  mutated EMP-1- C6G, C15G
GGTYSGHFGPLTWVGKPQGG

SEQ ID NO: 29 - DNA, 140 bp, Artificial Sequence
  mutated His289-Gly290 insert PCR product (Cys to Gly)
agacaaatcaaaagaatttcaactattcagctctcctcatggtggtacttactctggtcattttggtccattgacttgggttggtaagccacaaggtggtgggaaggacctgctgtttaaggactctgcccacgggtttt

SEQ ID NO: 30 - DNA, 210 bp, Artificial Sequence
  mutated Glu625-Thr626 insert PCR product (Cys to Gly)
cctatttggaagcaacgtaactgactgctcgggcaacttttgtttgttccggtcggaaggtggtacttactctggtcattttggtccattgacttgggttggtaagccacaaggtggtaccaaggaccttctgttcagagatgacacagtatgtttggccaaacttcatgacagaaacacatatgaaaaatacttaggagaagaatatgt

SEQ ID NO: 31 - protein, 10 aa, Artificial Sequence
  putative EMP-1 peptide
  MISC_FEATURE (1)..(1): May be Cys
  MISC_FEATURE (2)..(2): May be Arg, His, Leu or Trp
  MISC_FEATURE (3)..(3): May be Met, Phe or Ile
  MISC_FEATURE (6)..(6): May be any of the 20 genetically encoded L-amino acids
  MISC_FEATURE (9)..(9): May be Asp, Glu, Ile, Leu or Val
  MISC_FEATURE (10)..(10): May be Cys
XXXGPXTWXX

SEQ ID NO: 32 - protein, 6 aa, Artificial Sequence
  peptide linker
PEAPTD

SEQ ID NO: 33 - protein, 25 aa, Artificial Sequence
  peptide linker
AEPKSCEKTHTCPPCPAPELLGGPS

SEQ ID NO: 34 - protein, 20 aa, Artificial Sequence
  mutated EMP-1
GGTYSSHFGPLTWVSKPQGG

SEQ ID NO: 35 - protein, 20 aa, Artificial Sequence
  mutated EMP-1
  MISC_FEATURE (6)..(6): May be any other amino acid but Cys
  MISC_FEATURE (15)..(15): May be any other amino acid but Cys
GGTYSXHFGPLTWVXKPQGG

SEQ ID NO: 36 - DNA, 330 bp, Artificial Sequence
  EPM encoding nucleotide sequence of pREX0387
tctcaaccaggcccaggaacattttggcaaagacaaatcaaaagaattccaactattcggtggtacttactcttgtcattttggtccattgacttgggtttgtaagccacatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacag

SEQ ID NO: 37 - DNA, 79 bp, Artificial Sequence
  P0141 primer
ggtggtacttactcttgtcattttggtccattgacttgggtttgtaagccacatgggaaggacctgctgtttaaggact

SEQ ID NO: 38 - DNA, 80 bp, Artificial Sequence
  P0142 primer
tggcttacaaacccaagtcaatggaccaaaatgacaagagtaagtaccaccgaatagttggaattcttttgatttgtctt

SEQ ID NO: 39 - DNA, 20 bp, Artificial Sequence
  P0011 primer
tacacagcttacagagactg

SEQ ID NO: 40 - DNA, 20 bp, Artificial Sequence
  P0031 primer
tactgtgacttacctgagcc

SEQ ID NO: 41 - DNA, 904 bp, Artificial Sequence
  nucleotide encoding EPM peptide of pREX0155
aggtctctagagaaaagggtacctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatggaacatattcatgtcacttcggtcctttaacatgggtatgtaaacctcaactgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactat

SEQ ID NO: 42 - DNA, 81 bp, Artificial Sequence
  P0143 primer
ggaacatattcatgtcacttcggtcctttaacatgggtatgtaaacctcaactgtgtcaactgtgtccagggtgtggctgc

SEQ ID NO: 43 - DNA, 80 bp, Artificial Sequence
  P0144 primer
ttgaggtttacatacccatgttaaaggaccgaagtgacatgaatatgttccatccgcacaaggggcacagctgcccgaga

SEQ ID NO: 44 - DNA, 40 bp, Artificial Sequence
  P0101 primer
catgtctaagcttattattcatctgttggggcttctgggc

SEQ ID NO: 45 - DNA, 21 bp, Artificial Sequence
  P0090 primer
caagctaaacctaattctaac

SEQ ID NO: 46 - DNA, 294 bp, Artificial Sequence
  nucleotide encoding EPM peptides of pREX0607
tcaaaagaattccaactattcggtggtacttactctggtcattttggtccattgacttgggttggtaagccacatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagt

SEQ ID NO: 47 - DNA, 903 bp, Artificial Sequence
  nucleotide encoding EPM peptide of pREX0242
tctaggtctctagagaaaagggtacctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatggaacatattcaggtcacttcggtcctttaacatgggtaggtaaacctcaactgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaa

SEQ ID NO: 48 - protein, 17 aa, Artificial Sequence
  mutated EMP-1 peptide
GGTYSGHFGPETWVGKP

SEQ ID NO: 49 - protein, 17 aa, Artificial Sequence
  mutated EMP-1 peptide
GGTYSGHFGPLTWEGKP

SEQ ID NO: 50 - protein, 17 aa, Artificial Sequence
  mutated EMP-1 peptide
GGTYSGHFGPETWEGKP

SEQ ID NO: 51 - protein, 17 aa, Artificial Sequence
  mutated EMP-1 peptide
GGTYSGHFGPTTWDGKP